(19) United States
(12) Patent Application Publication
(10) Pub. No.: US 2005/0037450 A1
(43) Pub. Date: Feb. 17, 2005

(54) Novel targets for lithium therapy

(75) Inventors: John D. York, Chapel Hill, NC (US); Bryan David Spiegelberg, Nashville, TN (US)

Correspondence Address: JENKINS & WILSON, PA, 3100 TOWER BLVD, SUITE 1400, DURHAM, NC, 27707, US

(73) Assignee: Duke University

(21) Appl. No.: 10/635,265

(22) Filed: Aug. 6, 2003

Related U.S. Application Data
(60) Provisional application No. 60/401,480, filed on Aug. 6, 2002.

(51) Int. Cl. C12Q 1/42

(52) U.S. Cl. 435/21

(57) ABSTRACT

Compounds for use as lithium-like therapeutic agents and methods and reagents for identifying same as well as compounds for treating lithium-induced toxicity and methods and reagents for identifying same. The compounds modulate the activity of enzymes within pathways upon which lithium has been discovered to act. Also disclosed herein are transgenic animals that serve as models of lithium-induced toxicity and methods of using the transgenic animals for identifying compounds that ameliorate lithium toxicity. Furthermore, disclosed herein are methods of modeling target sites for lithium.

### RELATED APPLICATIONS

This application claims the benefit of U.S. Provisional Patent Application Ser. No. 60/401,480, filed Aug. 6, 2002, the disclosure of which is incorporated herein by reference in its entirety.

### GOVERNMENT INTEREST

This invention was made with Government support under Grant Nos. R01HL55672-05 and R01HL55672-06 awarded by National Institutes of Health. The Government has certain rights in the invention.

### TECHNICAL FIELD

Presently disclosed herein, in general, are compounds for use as lithium-like therapeutic agents and methods and reagents for identifying same as well as compounds for treating lithium-induced toxicity and methods and reagents for identifying same. More particularly, disclosed herein are compounds, and methods for identifying same, that modulate the activity of enzymes within pathways upon which lithium has been discovered to act. Also disclosed herein are transgenic animals that serve as models of lithium-induced toxicity and methods of using the transgenic animals for identifying compounds that ameliorate lithium toxicity. Furthermore, disclosed herein are methods of modeling target sites for lithium, and related compounds on lithium sensitive molecules for identifying additional compounds capable of binding the target sites.

### Table of Abbreviations

1ptase, INPP inositol polyphosphate 1-phosphatase
AP ammonium phosphate
APS adenosine 5′-phosphosulfate
AQP2 aquaporin-2
AVP arginine vasopressin
BHMT betaine-homocysteine methyltransferase
BPntase, BPNT bisphosphate 3′-nucleotidase
BSA bovine serum albumin
CM complete minimal media
DMSO dimethyl sulfoxide
EST Expressed Sequence Tag
Fbpase, FBP fructose 1,6-bisphosphate 1-phosphatase
FBS fetal bovine serum
GFP green fluorescent protein
GSK glycogen synthase kinase
GST glutathione S-transferase
IBMX 3-isobutyl-1-methylxanthine
IMCD inner medullary collecting ducts
Impase, IMPA inositol monophosphatase
Ins(1,3,4)P₃ inositol (1,3,4) trisphosphate
Ins(1,4)P₂ inositol (1,4) bisphosphate
Ins(1,4,5)P₃ inositol (1,4,5) trisphosphate
LB rich media (bacteria)
NDI nephrogenic diabetes insipidus
PAP 3′-phosphoadenosine 5′-phosphate
PAPS 3′-phosphoadenosine 5′-phosphosulfate
PAPSS PAPS synthetase
PDB The Protein Data Bank
PI(4,5)P₂ phosphatidyl inositol (4,5) bisphosphate
PLC phospholipase C
PMSF phenylmethylsulfonyl fluoride
PST phenol (aryl) sulfotransferase
RT-PCR Reverse Transcription-Polymerase Chain Reaction
SDS-PAGE sodium dodecyl sulfate-polyacrylamide gel electrophoresis
Sf9 Spodoptera frugiperda
YPD rich media (yeast)

### BACKGROUND

I. Therapeutic Uses of Lithium

Lithium and the salts thereof have been used to treat a variety of disorders. The initial medicinal use of lithium salts was in the treatment of gout (Cade, 1970). The mechanism of this function relied on the relative solubility of lithium urate, leading to the dissolution of urate deposits in the cartilage. The efficacy of lithium in the treatment of viral disorders has also been suggested (Jefferson, 1990). Lithium has been shown to inhibit the replication of DNA viruses such as those in the herpes simplex family (Rybakowski, 2000; Skinner, 1980). Accordingly, a lithium ointment was developed and was shown to improve the status of patients suffering from genital herpes (Skinner, 1983).

The most prevalent uses of lithium are in the treatment of acute or chronic bipolar disorder and in the prevention of bipolar disorder recurrence in individuals who have experienced transient episodes. Bipolar disorder is estimated to affect approximately one percent of people throughout the world (Woods, 2000). The disease is characterized by alternating episodes of mania and depression. Bipolar disorder, also known as manic depression, can lead to unpredictable behavior and is associated with an increased risk of suicide. One quantitative measure of the importance of a disease is the economic impact it has on society. Wyatt and Henter report that bipolar disorder results in over $45 billion in costs to society in the United States when such factors as medication, lost wages, substance abuse, and institutionalization are taken into account (Wyatt, 1995).

Many professional associations recommend lithium as the first treatment option for patients suffering with bipolar disorder (reviewed in Goldberg, 2000). Approximately half of all patients for whom lithium is prescribed will experience a diminution of their symptoms (Nemeroff, 2000). Further improvements in the results obtained with lithium therapy are gained through the combination of lithium with other anti-bipolar agents (Bowden, 2000). In the field of non-bipolar psychological disorders, lithium has been used to treat maladies ranging from alcoholism to unipolar depression (for review see Bowden, 2000). In the treatment of these psychological disorders, lithium is often prescribed as an augmentation of therapy when a patient is unresponsive to conventional treatment regimens.

The broad applicability of lithium across a spectrum of disorders is due, in part, to its pleiotropic effects on numerous mammalian organs, including the brain, kidneys, and other major organs. Davson et al., have suggested that the effect of lithium on magnesium transport through the choroid plexus structure of the brain might be a factor in the attenuation of mood disorders (Davson, 1987). Lithium has been shown to cause a significant rise in the concentration of magnesium in the plasma due to effects on choroid plexus transport (Birch, 1973; Reed, 1980). In addition, structural studies of the brains of patients suffering from bipolar disorder have shown a correlation between the disease and ventricular volume (Chen, 2000; Drevets, 1997; Sheline, 1999; Sheline, 1996; Soares, 1997). Because the volume of the brain is held constant by the rigid support of the skull, an increased ventricular volume means that the volume of the hippocampus and other structures are decreased. Lithium has been shown to alleviate this effect (Chen, 2000).

II. Lithium Toxicity

In addition to its positive effects on mood disorders and other human ailments, lithium exhibits a plethora of negative effects on human systems. Thus, despite the efficacy and relatively low cost of lithium treatment, alternatives are constantly being sought. Lithium is associated with numerous side effects, including nausea, diarrhea, and kidney dysfunction. In addition, continuous monitoring of serum lithium concentrations is required, because overdose can quickly lead to coma and death (Bowden, 2000). These side effects lead to problems with a lack of adherence to recommended therapeutic regimens in a large percentage of patients (Scott, 2002). In its most drastic manifestation, lithium can cause death in a variety of ways. Overdose can induce a shutdown of the nervous system, leading to coma and brain death. Lithium can also cause death through the induction of organ failure, particularly in susceptible patients such as the elderly and people with pre-existing heart and kidney disease.

More benign and yet more common side effects include afflictions of the kidneys, the gastrointestinal tract, and the thyroid (for review, see Schou, 2001). Urinary-concentrating defects arising in the kidney are common complaints among patients undergoing lithium therapy. Up to 20% of patients report clinically significant polyuria, in which daily urinary output can reach 10 L or more (Boton, 1987). Secondary to this effect is polydipsia, in which excessive thirst forces patients to consume vast quantities of liquid to maintain body fluid levels in the face of such high urine volumes. Acquired nephrogenic diabetes insipidus (NDI) is a hallmark of lithium treatment, occurring in 20-50% of patients taking the drug (Boton, 1987). Lithium-induced NDI is thought to arise from an interaction of the drug with the vasopressin (AVP)-activated adenylate cyclase system in the collecting ducts of the kidney (Christensen, 1985; Goldberg, 1988; Jackson, 1980; Yamaki, 1991).

Lithium treatment is also associated with the occurrence of diarrhea (Gelenberg, 1989). However, one reported pharmacological effect of lithium is in the prevention of secretory diarrhea arising from diverse causes (Donowitz, 1986). Oral lithium carbonate therapy has been reported to improve the status of patients suffering from diarrhea due to pancreatic cholera (Pandol, 1980) and diarrhea of unknown etiology (Owyang, 1984). The result of lithium therapy has been attributed to inhibition of the generation of cAMP (Owyang, 1984; Pandol, 1980). Another trial attempting to treat the diarrhea associated with pancreatic cholera syndrome resulted in exacerbation of the symptoms (Graham, 1980; Graham, 1975), indicating that diarrhea does not universally respond to lithium therapy.

Finally, a small percentage of those taking lithium experience hypothyroidism and its associated symptoms (Dwight, 2002; Henry, 2002).

Despite the risks associated with lithium treatment, it continues to be widely prescribed as a treatment for numerous disorders. For example, an extensive review of the use of lithium in the recent past concluded that it “continues to set a standard that has yet to be met by any proposed alternative mood-stabilizing treatments” (Baldessarini, 2002).

III. Mechanisms Of Lithium Action: Signaling Pathways

In light of its multitude of effects on biological systems, a vast body of literature has arisen concerning the molecular basis for the outcomes of lithium treatment. However, a complete mechanism of action for lithium, both therapeutic and toxic, has yet to be determined. Such an understanding would be invaluable for treating toxic side effects of lithium and developing alternative treatments to lithium therapy.

One signaling pathway known to be affected by lithium therapy is the inositol signaling pathway. Allison and Stewart showed in 1971 that chronic treatment of rats caused a depletion of free inositol from brain slices taken from these animals (Allison, 1971) with a corresponding increase in the concentration of inositol 1-phosphate (Allison, 1976a). Free inositol is the central player in the ubiquitous inositol signaling cycle, which involves modulation of the phosphorylation states of both lipid-bound phosphoinositides and soluble inositol phosphates (reviewed in Irvine, 2001). Lipid kinases and phosphatases create a variety of phosphoinositides whose free hydroxyl groups are phosphorylated in all positions in a number of combinations. These phosphorylated lipid molecules play important roles in their own right, binding to numerous protein modules. In addition, a multitude of cellular stimuli result in the activation of phosphoinositide-specific phospholipase C (PLC), which hydrolyzes phosphoinositide (4,5)-bisphosphate (PI(4,5)P₂)) to form inositol (1,4,5)-trisphosphate (Ins(1,4,5)P₃) and diacylglycerol. Diacylglycerol is a secondary signaling molecule with several roles, including the activation of protein kinase C. The second messenger Ins(1,4,5)P₃ has been focused on primarily in its role in the stimulation of calcium release from internal stores.

Despite the focus on calcium release, soluble phosphorylated inositols play numerous other signaling roles. This is emphasized by the fact that more than 25 phosphorylated forms of inositol have been identified in various cellular extracts (Irvine, 2001). In order to terminate phosphorylated-inositol-mediated signals and to restore inositol into the membrane for potentiation of PLC-mediated signaling, polyphosphorylated inositols are dephosphorylated by a series of enzymes to generate free inositol. Free inositol is combined with CDP-diacylglycerol to form phosphatidyl inositol, where it can continue in the inositol cycle.

This model of inositol signaling helped to explain the lithium-induced depletion of free inositol of chronically treated rats when it was found inositol monophosphatase (Impase) and inositol polyphosphate 1-phosphatase (1ptase), two enzymes involved in inositol dephosphorylation, were potently inhibited by lithium (Gee, 1988; Hallcher, 1980; Inhorn, 1988; Naccarato, 1974). These observations led to the proposal of the “inositol depletion hypothesis” (Berridge, 1989). In this hypothesis, lithium causes a depletion of free inositol through its interaction with Impase and 1ptase. The brain would be particularly sensitive to depletion of free inositol through the inhibition of the inositol phosphatases since the blood-brain barrier prevents the polar inositol molecule from entering brain tissues (Spector, 1975). Consequently, the major routes of free inositol generation in the brain are through recycling of higher phosphorylated inositols and de novo synthesis (Wong, 1987), each of which relies on Impase activity. The depletion of free inositol might dampen phosphatidyl inositol-based signaling, particularly in the overstimulated neurons of patients suffering from bipolar disorder (Berridge, 1989). Indeed, research has shown that lithium has an effect on stimulated generation of IP₃ and calcium responses (Atack, 1995).

An intriguing aspect of this hypothesis is that it could explain why lithium exerts differential effects on the neural activity of normal patients and patients suffering from bipolar disorder (Baraban, 1994; Berridge, 1989). The inhibitions of Impase and 1ptase by lithium are uncompetitive with respect to substrate, meaning that the potency of inhibition increases as the concentration of substrate increases. In some populations of the hyperexcited neurons of bipolar patients, inositol signaling might be overactive, leading to higher intracellular concentrations of phosphorylated inositols (Berridge, 1989). Thus, since more polyphosphorylated inositols are present in neurons of people suffering from bipolar disorder, Impase and 1ptase would be expected to be more sensitive to lithium relative to a normal environment (Baraban, 1994; Berridge, 1989). Further support of Impase as a relevant target of lithium came with the identification of a link between the genomic location of Impase and a region implicated in susceptibility for bipolar disorder (Yoshikawa, 2000; Yoshikawa, 1997).

Recently, a study by Williams et al. used a comparison of three drugs employed in anti-bipolar therapy to bolster the case for inositol signaling as a molecular target of lithium (Williams, 2002). Carbemazepine and valproic acid are two common alternatives that are utilized for patients in which lithium therapy does not achieve the desired results. Each of the three drugs had several effects on the properties of cultured sensory neurons from the dorsal root ganglia of neonatal rats (Williams, 2002). However, the authors focused on one common effect: an alteration of the characteristics of growth cones (Williams, 2002). The commonality of this effect on the neurons suggested that it could be relevant to the treatment of bipolar disorder (Williams, 2002). In that the alterations of growth cones were reversed by supplementation of the media with free inositol, it was proposed that inositol polyphosphates were involved in the response, and could thus be involved in the therapeutically-relevant response of neurons to the three anti-manic drugs (Williams, 2002).

Low concentrations of lithium and high concentrations of normally catalytic magnesium inhibit Impase and 1ptase uncompetitively with respect to substrate. The inhibitory effects of lithium and magnesium are mutually exclusive in Impase, implying that the inhibitory metals bind at the same or overlapping sites (Ganzhorn, 1990). Structural studies of 1ptase offered additional evidence. When the crystals of bovine 1ptase were grown in the presence of gadolinium, lithium, and substrate, the resulting electron density revealed that gadolinium bound at only one of the two metal sites observed in the original structure (York, 1994b). This was interpreted to mean that electron-poor lithium was bound at the second metal binding site, excluding the binding of electron-rich gadolinium, leading to a dearth of electron density at this site. This conclusion was supported by mutagenic data in which a residue that anchors the second metal binding site, Asp-54 (homologous to Impase Asp-47 (FIGS. 1 and 2)), was mutated to alanine. This mutant 1ptase had an affinity for lithium close to three orders of magnitude lower than that of the wild type enzyme.

Based on kinetic and structural data, Pollack et al. proposed the following mechanism of catalysis and lithium inhibition for Impase (Pollack, 1994). One Mg²⁺ ion binds at a high affinity site throughout the catalytic cycle. Following substrate binding, a second Mg²⁺ binds at a site with lower intrinsic affinity. When Mg²⁺ is present in both sites (and possibly a third metal binding site), hydrolysis occurs, and product and the Mg²⁺ in the low affinity site are released. When Li⁺ is present at sufficient concentrations, it binds at the lower affinity site following substrate binding. Even though substrate can be hydrolyzed and the dephosphorylated portion of the substrate can be released, Li⁺ remains bound at site 2, and the enzyme stays in an unproductive enzyme-Mg²⁺—PO₄²⁻—Li⁺ quaternary complex.

In spite of indications that Impase is a therapeutic target of lithium, conclusive evidence confirming the link between inositol signaling and lithium's mechanism of action has yet to surface. As reviewed by Jope and Williams, questions concerning the ability of lithium to affect concentrations of inositol and other components to a clinically relevant degree have arisen (Jope, 1994). Although numerous studies demonstrated the capacity of lithium treatment to reduce free inositol concentrations in vivo, the lithium doses used were relatively high and approached levels that would be toxic over the course of normal lithium therapy (Allison, 1976a; Allison, 1976b; Hallcher, 1980; Sherman, 1981; Sherman, 1985). Moreover, when animal models were treated with lithium chronically rather than acutely, the decreases of free inositol levels were less significant (Hirvonen, 1991; Sherman, 1981; Sherman, 1985; Whitworth, 1989), again questioning the applicability of the inositol depletion hypothesis to the molecular mechanism of lithium (Jope, 1994). Finally, the possibility that lithium-mediated inhibition of Impase and 1ptase can slow flux through the phosphatidyl inositol cycle was called into question by studies that showed that concentrations of Ins(1,4,5)P₃ and phosphoinositide phosphates in animal models were unaffected (Honchar, 1989; Jope, 1992; Lopez-Coronado, 1988; Whitworth, 1990).

IV. Characterization of Lithium-Sensitive Enzyme Family Members

The inability of Impase inhibition to completely explain the therapeutic effects of lithium implies that multiple targets are responsible for the biological effects of the drug, and therefore a complete mechanism of action is yet to be elucidated. The potent effect of lithium on Impase and 1ptase suggested that these related proteins might be part of a larger family of enzymes linked by a biochemical sensitivity to lithium. Therefore, a comparison of Impase and 1ptase was initiated to try to identify a common thread that could be used to identify other members of this hypothetical ‘lithium-sensitive’ family (Neuwald, 1991). Unfortunately, a simple overlay of the amino acid sequences of these enzymes did not identify a reasonable amount of similarity. It did, however, allow the recognition of a six amino acid motif, DP(i/l)D(s/g)T (also referred to as “DPIDST”), that was common to both enzymes (Neuwald, 1991). A third enzyme, fructose 1,6-bisphosphate phosphatase (Fbpase), was found to contain a similar motif, “DPLDGS” (Neuwald, 1991). Interestingly, previous biochemical analyses showed that Fbpase was sensitive to sub-millimolar concentrations of lithium (Marcus, 1980; Nakashima, 1976).

Numerous structures of potential lithium sensitive family members from humans and other organisms have now been solved at high resolution. The structures are made up of similar mixed α/β folds (FIG. 3). In each case, the ‘DPIDST’ motif has been localized to the active site of the enzyme. Moreover, despite an apparent lack of an evolutionary relationship based on the amino acid sequences, an overlay of the structures, anchored at the ‘DPIDST’ sequence, revealed that the core structures of 1ptase, Impase, and Fbpase are very similar (York, 1995).

In fact, a common core structure of approximately 155 amino acids emerged, with the α-carbon backbones superimposing to within 3 Å root-mean-squared deviation (York, 1995). The importance of this finding was twofold. First, the structural similarity among the enzymes suggested that the proteins did share a common evolutionary ancestor, supporting the idea of a ‘family’ of enzymes that held lithium sensitivity as a common characteristic. Second, the structural comparison helped to expand the ‘DPIDST’ sequence into a stringent sequence motif that united the family.

However, structural characterization of the lithium sensitive family emphasized that the ‘DPIDST’ motif alone was insufficient to define family membership. For example, the beta subunit of the ATPase F1 β subunit (GenBank accession number PO₆₅₇₆) contains a similar motif, DPLDST (York, 1995). The structure of this enzyme has been solved (Abrahams, 1994), and does not show structural homology with members of the family (York, 1995). Importantly, while the ATP synthetase contains the ‘DPIDST’ motif, it did not contain the expanded motif, supporting the importance of the structural studies in developing a stringent criterion that can be used to identify enzymes in the lithium sensitive family.

The use of three-dimensional protein structure analysis permitted the conclusive identification of lithium-sensitive family members such as Fbpase, where such an identification was not possible using sequence similarity local alignment strategies. Comparison of three-dimensional structures has resulted in the identification of a signature motif of this family, referred to herein alternatively as the “lithium sensitivity motif”, “common motif” or the “unifying motif”, as follows:

| | |
|---|---|
| D-Xₙ-EE-Xₙ-DPiDgtXₙwd-X₁₁- | (FIG. 1; SEQ ID NO: 3) |
| GG | |

where X is any amino acid, n is any integer; D is aspartic acid E is glutamic acid; P is proline; G is glycine; i is isoleucine or an amino acid that can be conservatively substituted in place thereof; g is glycine or an amino acid that can be conservatively substituted in place thereof; t is threonine or an amino acid that can be conservatively substituted in place thereof; w is tryptophan or an amino acid that can be conservatively substituted in place thereof; and d is aspartic acid or an amino acid that can be conservatively substituted in place thereof.

In humans and other mammals, there are seven known open reading frames that contain the lithium sensitivity motif (Table 1, FIG. 4).

TABLE 1
Substrate specificities of known members of the lithium-sensitive
family. qa-x is most similar to human Impase (47% similar,
BLAST score 4 × 10⁻²⁷). LPM is most similar to human BPntase
(45% similar, BLAST score 2 × 10⁻²⁰). LPM has been identified
as an isoform of Impase (IMPA3) (Parthasarathy, 2001) despite a
lack of supporting experimental evidence.

| | | | Representative Acc. |
|---|---|---|---|
| Enzyme | Substrate(s) | Organisms | No. |
| Impase | monophosphorylated | mammals, fungi | P29218 |
| | inositols | | |
| 1ptase | Ins(1,4)P₂, | Mammals | P49441 |
| | Ins(1,3,4)P₃ | | |
| Fbpase | Fru(1,6)P₂ | mammals, fungi, | P09467 |
| | | bacteria | |
| BPntase | bisphosphorylated | mammals, fungi, | NM_006085 |
| | nucleotides | bacteria | |
| LPM | unknown | Mammals | AY032885 |
| Qa-X | unknown | Fungi | B31277 |
| | Ins(1)P, | other | |
| suhB | monophosphorylated | Bacteria | P22783 |
| | substrates | | |
| MJ0109 | Fru(1,6)P₂, Ins(1)P | Archaea | E64313 |

Numerous studies have shown that lithium binds the active sites of these enzymes. Amino acids in the common motif have been shown to be essential to inhibition by lithium, implying that the common motif not only links lithium sensitive enzymes but is essential to the interaction of lithium with these enzymes as well. It appears likely that the active site of enzymes of the lithium sensitive family has evolved as an effective tool for effecting the hydrolysis of small phosphorylated molecules in a magnesium-dependent manner, and that this collection of amino acids constitutes a unique geometry that accommodates the strong binding of the non-physiological ion lithium.

The lithium-sensitive family has emerged as an attractive set of enzymes that could hold the key to unlocking the enigma that constitutes lithium therapy (York, 1995). Most importantly, each of the mammalian enzymes that have been described is inhibited by lithium at a therapeutically relevant concentration. Also important to a mechanism with members of the lithium-sensitive family at its core is the fact that the proteins are involved in numerous distinct biological pathways.

For example, Fbpase is a central enzyme in gluconeogenesis. The enzyme hydrolyzes fructose 1,6-bisphosphate to form fructose 6-phosphate and inorganic phosphate. Fructose 6-phosphate is reversibly transformed by phosphoglucose isomerase to form glucose 6-phosphate, which is dephosphorylated by glucose 6-phosphatase to form glucose. Fbpase exists as a tetramer that can be regulated in a competitive or allosteric manner by such molecules as 5′ AMP, fructose 2,6-bisphosphate, and citrate in accordance with the needs of the brain and other organs for glucose. Lithium has been found to alter gluconeogenesis in renal tissues (Stepinski, 1984), pointing to a potential role for Fbpase in the physiological effects of lithium.

Bovine 1ptase, another lithium-sensitive family member, was originally described by Inhorn et al. (Inhorn, 1987a; Inhorn, 1987b; Inhorn, 1988). Subsequently, the human enzyme was cloned (York, 1993), and the three dimensional structure of the bovine enzyme was solved (York, 1994a; York, 1994b). In cultured cells, 1ptase was found in the nucleus (York, 1994c). Interestingly, the 1ptase substrate Ins(1,4)P₂ had been found to bind to and activate DNA polymerase α (Sylvia, 1988). Indeed, transient expression of 1ptase caused a decrease in DNA replication, possibly through hydrolysis of the DNA polymerase activator (York, 1994c). A direct link between 1ptase and lithium therapy came with the discovery by Acharya et al. that disruption of 1ptase in Drosophila resulted in synaptic vesicle defects that were phenocopied by lithium administration (Acharya, 1998).

A crystal structure of rat bisphosphate 3′-nucleotidase has been solved (Patel, 2002). As predicted according to its membership in the family, the enzyme shares the identical α/β/α/β fold (Patel, 2002). Further analysis indicated that the enzyme retained the core structure, for example overlaying with the structure of 1ptase to 1.66 Å RMS deviation (Patel, 2002). However, the properties of bisphosphate 3′-nucleotidase and its interactions with lithium have heretofore not been fully elucidated.

BPntase removes the 3′-phosphate from 3′,5′-bisphosphate nucleosides and 3′-phosphoadenosine 5′-phosphosulfate with Kₘ and Vₘₐₓ values of 0.5 μM and 40 μmol/min/mg. BPntase is competitively inhibited by inositol 1,4-bisphosphate with a Kᵢ of 15 μM, and it has been suggested that the physiological role of BPntase in nucleotide metabolism is regulated by the inositol signaling pathways (Speigelberg, 1999).

Members of the lithium sensitive family are not restricted to expression in mammals (Table 1). Homologues of Fbpase and bisphosphate 3′-nucleotidase have been found in both bacteria and yeast. Lithium sensitive enzymes expressing Impase activity have been described in yeast (Lopez, 1999), and the bacterial enzyme suhB has been described as having inositol monophosphatase activity (Matsuhisa, 1995). The Neurospora crassa gene qa-x is predicted to encode a member of the lithium sensitive family (GenBank accession number X14603), but substrates have not been described. The sequence is similar to that of Impase, and the gene was found in a cluster of genes involved in quinone metabolism (Geever, 1989). Finally, discovery of a bifunctional Fbpase/Impase from Archaea was described recently (Johnson, 2001; Stec, 2000; Stieglitz, 2002).

V. The Sulfur Assimilation Pathway and Lithium Toxicity

Murguia et al. (Murguia, 1996) and Dichtl and Tollervey (Dichtl, 1998), among others, have reported that lithium toxicity in yeast is mediated through the production of PAP, a metabolite in the sulfur assimilation pathway. In this pathway, organisms increase the potential energy of inorganic sulfate (SO₄²⁻) by conjugating it to ATP (FIG. 5). Intracellular sulfate is first conjugated to ATP through an ATP sulfurylase enzyme activity to form 5′ APS. The equilibrium of this first reaction is pushed toward products by the phosphorylation of 5′ APS in an ATP-dependent manner with APS kinase. The product of this reaction, PAPS, has a high-energy phosphosulfate bond. The sulfate moiety in PAPS can be donated to biological molecules or reduced to sulfide, which is then incorporated into sulfur-containing amino acids. Sulfate donation results in the production of PAP, the substrate of bisphosphate 3′-nucleotidase activity and an inhibitor of numerous enzyme activities.

Alternatively, PAP could have a more direct effect on adenylate cyclase activity. PAP has been shown through biological and biochemical studies to affect the activity of numerous proteins, including the yeast Xrn1p (Dichtl, 1998), nucleoside diphosphate kinase (Schneider, 1998), and PAPS:PAP antiport systems (Ozeran, 1996a; Ozeran, 1996b). In addition, PAP has been shown to act as a P-site inhibitor of adenylate cyclase itself. The IC₅₀ for PAP's inhibition of preparations of native adenylate cyclase-containing membranes was found to be approximately 50 μM (Johnson, 1989). In yeast, PAP accumulates upon lithium treatment (elaborated in Detailed Description), suggesting that PAP could be a novel, physiologically-relevant P-site inhibitor of adenylate cyclase.

While the overall scheme is conserved, the details of the sulfur assimilation pathway differ among organisms. In yeast, creation of PAPS is accomplished by two proteins. The product of the MET3 gene is an ATP sulfurylase, while the product of the MET14 gene is an APS kinase. Since methionine is a more efficient source of sulfur than is inorganic sulfate, methionine (Cherest, 1971) or a metabolite (Paszewski, 1992) down-regulates the transcription of the MET3 and MET14 genes in yeast.

Degradation of PAP in yeast is mediated by HAL2p/MET22p, a member of the lithium-sensitive enzyme family. This enzyme, as predicted from the unifying motif, is sensitive to lithium at sub-millimolar concentrations (Glaser, 1993; Murguia, 1995). Murguía et al. showed via an HPLC analysis that lithium treatment caused an increase of intracellular PAP of at least 290-fold (Murguia, 1996).

Mutations in either HAL2/MET22 or cysQ result in defective sulfur assimilation (Neuwald, 1992; Thomas, 1992) providing additional evidence for the biologically relevance of PAPS nucleotidase activity. Neuwald et al. suggested that PAPS or a derivative might be cytotoxic when allowed to accumulate (Neuwald, 1992). In support of this hypothesis, it was shown that poor growth due to mutations in the PAPS-utilization pathway can be rescued by inhibiting the formation of PAPS with additional mutations in cysC, a 5′-APS 3′-kinase (Neuwald, 1992). Furthermore, Peng and Verma have shown that supplementation of media with methionine but not sulfite supports growth of hal2 mutants, indicating that the PAPS nucleotidase activity is most relevant (Peng, 1995).

Alternatively, due to their similar structures, PAP and PAPS might play cooperative roles in the sulfur assimilation pathway. For example, Ozeran et al. showed that a PAPS translocase transports PAPS across mitochondrial membranes via an antiport mechanism with PAP as the returning ligand (Ozeran, 1996a; Ozeran, 1996b).

In addition to a possible effect on sulfur assimilation, recent evidence points to a role of PAP nucleotidase activity in regulating RNA processing. Dichtl et al. (Dichtl, 1998) reported deletion of hal2 results in defects in Xrn1p-mediated RNA processing by due to direct inhibition by PAP. This enzyme is not essential, but the redundant function is accomplished by RNase MRP, an enzyme that might itself be inhibited directly or indirectly by lithium. Therefore, Dichtl et al. propose that lithium toxicity, at least in yeast, is mediated by inhibition of RNase MRP and by concurrent inhibition of the cytosolic enzyme Xrn1p via inhibition of HAL2 and subsequent PAP accumulation (Dichtl, 1998). Under growth conditions containing high Na⁺ or Li⁺ concentrations, overexpression of the PAP-metabolizing enzymes Hal2p and SAL1 would rescue growth by an increase in enzyme activity, thus reducing accumulated PAP pools. Methionine supplementation would also rescue growth by down-regulating the production of PAP from PAPS (Cherest, 1971).

In mammals, creation of PAPS occurs via bifunctional enzymes referred to as PAPS synthetases, in which ATP sulfurylase and APS kinase activities are present on the same polypeptide (Li, 1995). In mammalian cells, it has been shown that methionine supplementation does not result in a decrease in the intracellular concentration of PAPS (Kim, 1995).

It is increasingly apparent that lithium interacts with numerous intracellular targets. Accordingly, lithium has numerous effects on biological systems. Although this variety of effects makes lithium a desirable therapeutic, its non-specificity also results in numerous undesirable toxic side effects. Therefore, there is a long-felt need for a better understanding of the specific mechanisms of action of lithium, both therapeutic and toxic. This understanding would provide for the development of much needed models and screens for designing or discovering alternative compounds with greater specificity and fewer side effects than lithium. Alternatively, or in conjunction with new lithium-like acting compounds, knowledge of the molecular interactions of lithium within biochemical pathways would also provide new targets for alleviating the numerous side effects.

### SUMMARY

BPntase (SEQ ID NOs: 1 and 2), an exemplary PAP phosphatase and member of the lithium-sensitive family of enzymes, has been further characterized herein, and it has been discovered that the enzyme is enriched in tissues that are involved in fluid and ion transport in the kidney, the intestines, and the brain. Further localization of BPntase expression to the neurons indicates that this enzyme is involved in the therapeutic effects of lithium, while other presently disclosed studies indicate a role in the modulation of lithium toxicity as well. Moreover, chlorate has been identified as a compound that affects BPntase activity, inhibits production of PAP in vivo, and lowers the toxicity of lithium. These findings are generally applicable to compounds that interact with components of the sulfur assimilation enzyme pathway, and are useful in the identification and evaluation of compounds effective in the amelioration of lithium toxicity and/or as alternative treatments to lithium.

In view of the foregoing, methods of identifying a compound that modulates the activity of a sulfur assimilation pathway enzyme are provided. These methods generally comprise contacting a compound with a sulfur assimilation pathway enzyme. Similarly, methods for identifying a compound that modulates the activity of a PAP phosphatase enzyme are also provided. These methods generally comprise contacting a compound with a PAP phosphatase polypeptide, and detecting modulation of the activity of the PAP phosphatase polypeptide. In certain embodiments, the PAP phosphatase polypeptide is Bpntase (SEQ ID NO: 2). In yet other embodiments, the modulation of BPntase activity is detected in a recombinant yeast-based assay.

Transgenic non-human vertebrate animals having a modified gene encoding a BPntase polypeptide incorporated into their genome are also provided. These transgenic animals are useful as models of lithium treatment, models of lithium toxicity, and animal models for the screening of compounds useful in treating lithium toxicity.

In particular, methods of identifying a compound for treating a toxic effect resulting from a therapeutic treatment can comprise (a) obtaining a transgenic non-human vertebrate animal having incorporated into its genome a disruption of a gene encoding a BPntase polypeptide, wherein the disruption results in the transgenic animal exhibiting the toxic effect; (b) administering the compound to the transgenic animal; and (c) observing the transgenic animal for a change in the transgenic animal indicative of amelioration of the toxic effect.

Still other methods for identifying a compound that modulates the activity of a BPntase polypeptide (SEQ ID NO: 2) comprise modeling an interaction between the putative compound and a target moiety on the BPntase polypeptide. In certain embodiments, computer modeling is used as the modeling method.

Compounds identified by the foregoing screening methods are useful as alternative therapeutics to lithium treatment. Further, screens for compounds that modulate other enzymes acting within the sulfur assimilation pathway are likewise useful as alternative therapeutics to lithium treatment, as well as in treatments for lithium induced toxicity.

Accordingly, methods for treating lithium-related toxicity are provided herein. Exemplary methods comprise administering to a subject suffering from such toxicity a therapeutically effective amount of a compound that modulates the activity of at least one sulfur assimilation pathway enzyme.

It is accordingly an object of the present disclosure to provide methods for screening compounds that are useful in the treatment of lithium toxicity. It is also an object to provide methods for treating lithium-related toxicity. Still another object is the provision of transgenic animals that are useful models for lithium treatment and toxicity.

Some of the objects having been stated hereinabove, and which are addressed in whole or in part by the presently disclosed subject matter, other objects will become evident as the description proceeds when taken in connection with the accompanying Drawings and Examples as best described hereinbelow.

### BRIEF DESCRIPTION OF THE DRAWINGS

FIG. 1 is a comparison of the uniting sequence motif among three lithium sensitivity family members (SEQ ID NOs: 3-6). Members of the lithium family share a common sequence motif that was described based on a comparison of the three dimensional structures (York, 1995). Components of the consensus motif of human enzymes are indicated in bold lettering. The numbers above the amino acids indicate the sequence position of the first amino acid in the noted section of the motif. In the consensus sequence, absolutely conserved residues are shown in capital letters, while conservation of similarity is shown in lower case letters.

FIG. 2 is a detailed view of the active site of Impase. Coordinates for the high-resolution structure of human Impase are derived from Protein Data Bank (PDB) file 1IMD (Bone, 1994a). Displayed are amino acids in the consensus lithium sensitivity motif. Asp-47, Glu-70, Glu-71, Asp-90, the backbone oxygen of Ile-92, Asp-93, and Asp 220 coordinate the divalent cations required for catalysis (displayed as gray balls). The diglycine motif (Gly232 and Gly233) caps the helix containing Trp219 and Asp220 and is not shown in this view. See SEQ ID NO: 4. The figure was created with Swiss-PDBViewer (Guex, 1997) and Persistence of Vision Ray Tracer (Pov-Team, 2002).

FIG. 3 is a topology diagram of three members of the lithium sensitive family. The structures of human Impase (Bone, 1992), bovine 1ptase (York, 1994b), and ovine Fbpase (Choe, 1998) have conserved secondary structure elements. β strands are displayed as arrows and α helices are displayed as cylinders, and the N- and C-termini are labeled. The α helix and β strand labeled ‘F’ and shaded are unique to Fbpase, and the α helix labeled IV and shaded is absent in 1ptase. Residues corresponding to β strands 1 and 2 are disordered in the structures of Impase and 1ptase and have not been assigned secondary structure. The ‘DPIDST’ (See SEQ ID NOs: 3-6) consensus motif lies in β strand 4, which is labeled with an asterisk. The elements are not drawn to scale. (This figure was adapted from Patel, 2002).

FIG. 4 shows the evolutionary relationship of human members of the lithium sensitive family. A dendogram relating the sequences of the seven known human members of the lithium sensitive family reveals three classes. Models of the substrates of the noted enzymes are displayed near their names. Phosphate groups are shown as shaded circles, and the scissile phosphate is a darker shade. FBP isoforms hydrolyze the 1-position phosphate from fructose 1,6-bisphosphate. IMP isoforms hydrolyze the phosphate from monophosphorylated inositols. In the third related class, INPP hydrolyzes the 1-position phosphate from Ins(1,4)P₂ and Ins(1,3,4)P₃, BPNT hydrolyzes the 3′ position phosphate from 3′,5′-bisphosphorylated nucleotides, and the substrate or substrates for LPM are unknown.

FIG. 5 shows the sulfur assimilation pathway. To incorporate sulfur into biomolecules, organisms combine inorganic sulfate with ATP to form the high energy compound PAPS. The sulfate moiety on PAPS can be donated to lipids, proteins, and carbohydrates through a variety of sulfotransferases or reduced through PAPS reductases for subsequent incorporation into sulfur-containing amino acids. The product of these processes, PAP, is degraded to 5′ AMP by BPntase activity.

FIG. 6 shows that yeast and mammalian PAPS synthetase activities complement the methionine auxotrophy of met3Δ and met14Δ strains. Strains in which the MET3 or MET14 open reading frames had been deleted with a G418 resistance cassette were obtained from the Saccharomyces Genome Project. The deletions were complemented with yeast Met3p or Met14p or human PAPSS2 by expression on a 2μ plasmid with a galactose-inducible promoter. Following growth to mid-log phase in CM/ura⁻ containing 2% galactose, cells were washed and diluted to 1×10⁴, 2×10³, 400, and 80 cells per μl. One microliter of each dilution was plated on CM/ura⁻/met⁻ or CM/ura⁻ containing 2% galactose. The plates were imaged following growth at 30° C. for two days.

FIG. 7 is a graph showing met3Δ and met14Δ strains are lithium resistant. Wild type, met3Δ, and met14Δ yeast were resuspended at 5×10⁴/ml in CM/ura⁻ containing 2% dextrose and 1.25 μg/ml methionine. Growth at various concentrations of LiCl was measured after a 24 hr incubation at 30° C. The growth is plotted relative to growth in media lacking lithium. Growth inhibition of wild type yeast by lithium occurred with an IC₅₀ of approximately 40 mM, while the growth of met3Δ and met14Δ strains displayed an IC₅₀ of approximately 200 mM lithium.

FIG. 8 is a graph showing binding of PAP to GST-PST. Binding of radiolabeled PAP to 100 nM purified GST-PST was assayed in the presence of various concentrations of unlabeled PAP. Shown is a Scatchard plot of the resulting data, displayed as the ratio of bound to free PAP vs. the concentration of bound PAP in nM. The data are linear and intersect the y-axis at [Sb]/[Sf]=2.8. The calculated K_{d} is 35.7 nM.

FIG. 9 is a graph showing lithium-dependent accumulation of PAP mediated by yeast PAPS synthetase activities or hPAPSS2 is inhibited by chlorate. Yeast were grown in CM/ura⁻ containing 2% galactose to mid-log phase. The cultures were washed extensively and resuspended in the CM/ura⁻/met⁻ media with the noted inclusions. PAP was measured using the ligand binding assay described in Experimental Procedures. Intracellular PAP concentrations were normalized to the amount of soluble protein extracted from the cultures.

FIG. 10 is a graph showing methionine supplementation induces lithium resistance. Wild type yeast were inoculated at 5×10⁴ cells per ml into synthetic media with or without methionine containing the noted concentration of LiCl. Growth was assayed by spectrophotometry following 48 hrs of growth at 30° C. The IC₅₀ for lithium in met⁻ media was approximately 40 mM, while the IC₅₀ in met⁺ media was approximately 160 mM.

FIG. 11 is a graph showing chlorate reduces sensitivity of yeast growth to lithium treatment. Wild type yeast were grown in CM/ura⁻ containing 2% galactose to mid-log phase. The cultures were washed and resuspended at 5×10⁵ cells/ml in CM/ura⁻/met⁻ containing the noted lithium and chlorate concentrations. Following growth for 24 hr at 30° C., the cultures were resuspended, and growth was measured by determining the absorbance at λ=600 nm.

FIGS. 12A and 12B show Northern blot analysis of BPntase distribution in human tissues. (FIG. 12A) A multi-tissue Northern blot was obtained from Clontech and probed with a fragment of the human BPntase open reading frame. Mobilities of standard mRNA fragments are indicated by arrows (numbers in kb). The membrane was stripped and reprobed with a fragment specific to actin, results of which are displayed in the bottom panel of (FIG. 12A). The signal from the BPntase probe was normalized to the actin signal. A histogram of the normalized signals is displayed in panel (FIG. 12B).

FIG. 13 is a Western blot analysis of multiple mouse tissues. Tissues were dissected from freshly sacrificed mice, and crude extracts (20 μg) or elutions from PAP-agarose were analyzed by Western blotting with polyclonal anti-mBPntase antibodies. Lanes represent: (1) recombinant mBPntase (5 ng), (2) crude kidney, (3) crude lung, (4) crude heart, (5) crude liver, (6) elutions from PAP-agarose chromatography of kidney extract, (7) lung elutions, (8) heart elutions, and (9) liver elutions.

FIG. 14 is a Western blot showing BPntase expression throughout the kidney, including the inner medulla. Sections dissected from a rat kidney or cultured mIMCD-3 cells were lysed and analyzed via Western blotting with an anti-recombinant BPntase antibody and via PAP hydrolysis activity assays with 1 μM PAP. Lanes represent (1) recombinant BPntase (10 ng), (2) cortex (40 μg), (3) outer medulla (40 μg), (4) inner medulla (40 μg), and (5) mIMCD-3 (40 μg).

FIGS. 15A-15F is a series of photomicroscopic images showing BPntase localization throughout the mouse kidney, especially in the proximal tubules and thick ascending limb. (FIG. 15A) Low magnification overview of the kidney. Mouse kidney slices were probed with a 1:100 dilution of purified polyclonal anti-recombinant BPntase antibodies. Localization was visualized by probing with a peroxidase-conjugated secondary antibody and staining with DAB. (FIG. 15B) Higher magnification view of the boundary of the cortex and outer medulla. (FIG. 15C) Higher magnification view of the outer medulla. (FIG. 15D) Serial slice of the outer medulla probed with an anti-NKCC2 antibody. (FIG. 15E) Higher magnification view of the inner medulla. (FIG. 15F) Serial slice of the inner medulla probed with an anti-AQP2 antibody.

FIGS. 16A-16B are a series of photomicroscopic images showing BPntase distribution in the embryonic mouse. Slices of mouse embryos from days 15 (FIG. 16A) and 16 (FIG. 16B) post-coitus were obtained and stained with a 1:100 dilution of purified polyclonal anti-mouse BPntase antibodies. The most intense staining was observed in the gastrointestinal tract of the day 16 embryo (FIG. 16B, arrows).

FIGS. 17A-17D are a series of high magnification photomicroscopic images of BPntase staining of the embryonic kidney and gastrointestinal tract. Embryos displayed in FIGS. 17A-17D were imaged at 10-fold higher magnification. Panels display (FIG. 17A) day 16 kidney, showing staining of the immature tubules, (FIG. 17B) day 15 gastrointestinal tract, showing little staining of the intestinal epithelia, and (FIG. 17C) and (FIG. 17D) day 16 gastrointestinal tract. The large arrow in panel C indicates a dearth of staining in the crypts of Lieberkuhn relative to that in the tips of the villi (small arrow).

FIG. 18 is a Western blot showing expression of BPntase in the adult mouse gastrointestinal tract. Mice were sacrificed following approximately 12 hr of food deprivation. The intestines were removed and dissected into approximately 2.0 cm segments. Protein was extracted by Dounce homogenization and subjected to a Western blot. 10 μg of crude protein or 1.25 ng purified recombinant mouse BPntase was loaded, and the blot was probed with a 1:2000 dilution of anti-BPntase polyclonal antibody.

FIG. 19 is a Western blot showing BPntase expression in mouse brain and in rat cortical neurons. (1) Recombinant mBPntase (10 ng). (2) Whole mouse brain (40 μg). (3) Cultured rat cortical neurons (40 μg). Proteins were extracted with a Dounce homogenizer (lane 2) or by sonication (lane 3). The Western blot was probed with a 1:1000 dilution of polyclonal anti-mouse BPntase. Bisphosphate 3′-nucleotidase specific activities of the lysates were determined and are displayed in Table 2.

FIGS. 20A-20E are a series of photomicroscopic images showing an immunohistochemical investigation of BPntase distribution throughout the mouse brain. The brains of freshly sacrificed mice were dissected, and slices were probed with 1:100 dilutions of purified polyclonal anti-mouse BPntase antibodies. (FIG. 20A) A low magnification view shows staining of neuronal cell bodies throughout the organ and the choroid plexus (arrows). (FIG. 20B) and (FIG. 20C) Ten-fold higher magnification of the choroid plexus. (FIG. 20D) Higher magnification of neuronal cell bodies of the dentate gyrus in the hippocampus. (FIG. 20E) Neuronal cell bodies in the cortex.

FIG. 21 is a schematic map of BPntase gene, targeting construct and the recombinant allele of Example 5. The top panel represents the wild type BPntase genomic structure. Exons 3-6 are shown as filled boxes. The middle panel shows the targeting construct. The bottom panel is the recombinant allele after the targeting event. Exon 4 and 5 are replaced by PGK-Neo. The PCR primers Neo1 and BPN25 are shown as arrows. The Southern blot probes A and B are shown as shaded boxes.

FIGS. 22A and 22B depict mouse BPntase long arm genomic sequence (SEQ ID NO: 20) and BPntase short arm genomic sequence (SEQ ID NO: 17), as described in Example 5.

FIG. 23 is a diagram representing the Triple-Lox vectors for loxP/Cre targeted deletion. Triple-Lox base vectors were used for generation of the BPntase targeting vector as described in Example 5.

FIG. 24 summarizes the results of mutagenic and crystallography experiments representing the lithium binding site in 1ptase. An analysis of different electron density maps from crystallography studies determined that the electron density, when 1ptase was crystallized in the presence of metal, was seen at two sites (left panel). When 1ptase was crystallized in the presence of metal and lithium, however, the density at metal site 2 disappeared. This is explained by the fact that lithium, having only 2 electrons, is invisible (right panel). Therefore, loss of occupancy at metal site 2 is a result of invisible lithium binding there, excluding the binding of more electron rich metals. Mutagenic analysis of 1ptase at D54 to alanine resulted in a change in Ki for lithium from 0.5 M to 100 mM.

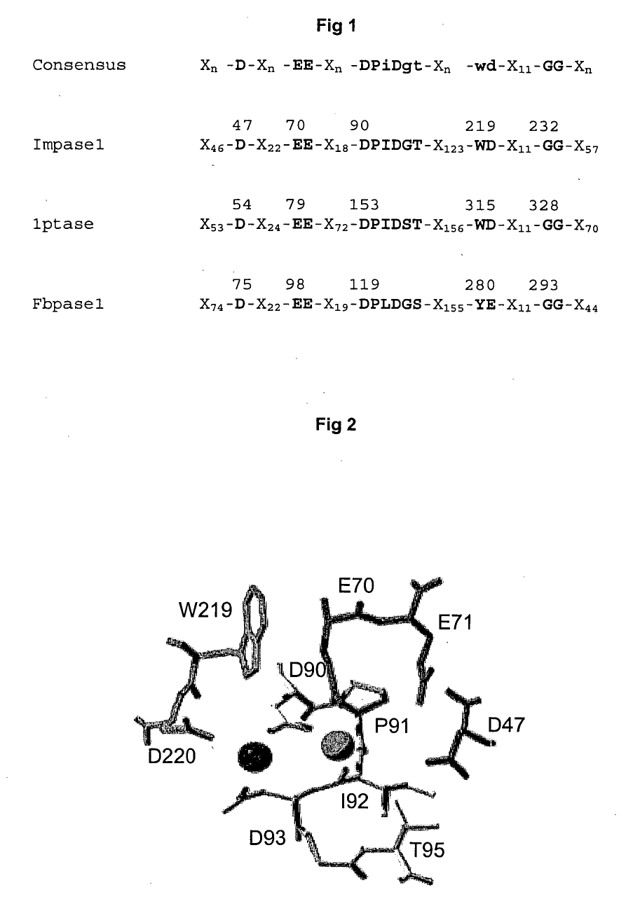

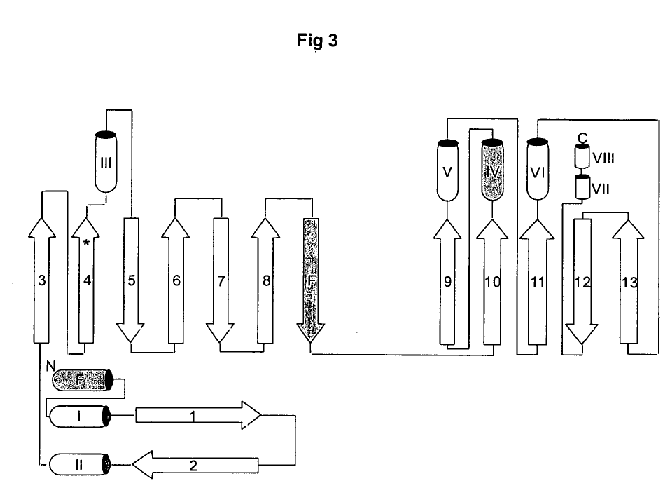

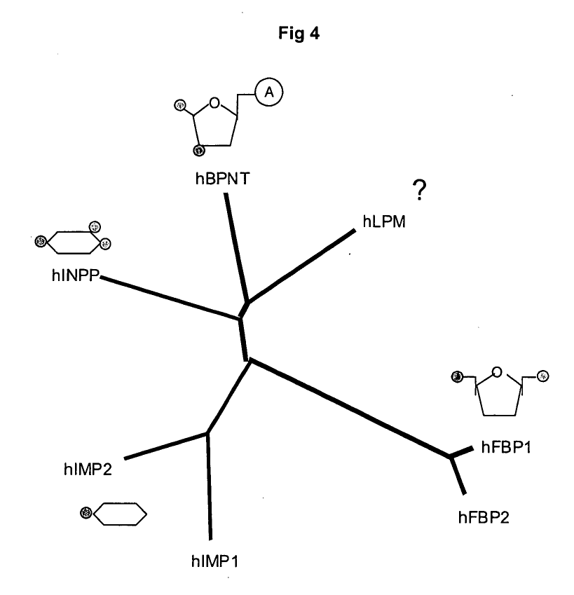

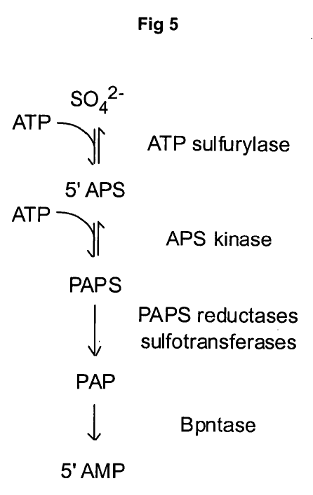

### DETAILED DESCRIPTION

The present subject matter will be now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the disclosure to those skilled in the art.

Unless otherwise defined, all technical and scientific terms used herein have the same meaning as commonly understood by one of ordinary skill in the art to which this disclosure belongs. All publications, patent applications, patents, and other references mentioned herein are incorporated by reference in their entirety.

I. Definitions

Following long-standing patent law convention, the terms “a” and “an” mean “one or more” when used in this application, including the claims.

The term “about”, as used herein when referring to a measurable value such as an amount of weight, time, dose, etc. is meant to encompass variations of ±20% or ±10%, more preferably ±5%, even more preferably ±1%, and still more preferably ±0.1% from the specified amount, as such variations are appropriate to perform the disclosed method.

As used herein, the term “biological effect” means any observable effect flowing from interaction between a polypeptide described herein and a ligand.

As used herein, the term “detecting” means confirming the presence of a target entity or event by observing the presence of a detectable signal, such as a radiologic or spectroscopic signal that will appear exclusively in the presence of the target entity or event.

As used herein, an “effective” amount or dose refers to one that is effective or falls within an effective range in at least some of a population of patients and that is sufficient to modulate a condition and/or to cause an improvement in symptoms in a subject.

The term “druggable region”, when used in reference to a polypeptide, nucleic acid, complex and the like, refers to a region of the molecule that is a target or is a likely target for binding a modulator. For a polypeptide, a druggable region generally refers to a region wherein several amino acids of a polypeptide would be capable of interacting with a modulator or other molecule. For a polypeptide or complex thereof, exemplary druggable regions including binding pockets and sites, enzymatic active sites, interfaces between domains of a polypeptide or complex, surface grooves or contours or surfaces of a polypeptide or complex which are capable of participating in interactions with another molecule. In certain instances, the interacting molecule is another polypeptide, which can be naturally occurring. In other instances, the druggable region is on the surface of the molecule. In one embodiment, a druggable region of an enzyme of the sulfur assimilation pathway comprises a lithium binding site.

As used herein, the term “gene” is used for simplicity to refer to a functional protein, polypeptide or peptide encoding unit. As will be understood by those in the art, this functional term includes both genomic sequences and cDNA sequences. Preferred embodiments of genomic and cDNA sequences are disclosed herein.

As used herein, the term “interact” means detectable interactions between molecules. The term “interact” is also meant to include “binding” interactions between molecules. Interactions can, for example, be protein-protein or protein-nucleic acid in nature.

As used herein, the term “modified” means an alteration from an entity's normally occurring state. An entity can be modified by removing discrete chemical units or by adding discrete chemical units. The term “modified” encompasses detectable labels as well as those entities added as aids in purification.

As used herein, the term “modulate” means an increase, decrease, or other alteration of any or all chemical and biological activities or properties of a polypeptide described herein. The term “modulation” as used herein refers to both upregulation (i.e., activation or stimulation) and downregulation (i.e. inhibition or suppression) of a response, and includes responses that are upregulated in one cell type or tissue, and down-regulated in another cell type or tissue.

As used herein, the term “mutation” carries its traditional connotation and means a change, inherited, naturally occurring or introduced, in a nucleic acid or polypeptide sequence, and is used in its sense as generally known to those of skill in the art.

A “knock-out” of a gene means an alteration in the sequence of the gene that results in a, decrease of function of the target gene, preferably such that target gene expression is undetectable or insignificant. A knock-out of an endogenous BPntase gene means that function of the BPntase gene has been substantially decreased so that expression is not detectable or only present at insignificant levels. “Knock-out” transgenics can be transgenic animals having a heterozygous knock-out of the BPntase gene or a homozygous knock-out of the BPntase gene. “Knock-outs” also include conditional knock-outs, where alteration of the target gene can occur upon, for example, exposure of the animal to a substance that promotes target gene alteration, introduction of an enzyme that promotes recombination at the target gene site (e.g., Cre in the Cre-lox system, for review see Sauer, 1998), or other method for directing the target gene alteration postnatally.

A “knock-in” of a target gene means an alteration in a host cell genome that results in altered expression (e.g., increased (including ectopic)) of the target gene, e.g., by introduction of an additional copy of the target gene, or by operatively inserting a regulatory sequence that provides for enhanced expression of an endogenous copy of the target gene. “Knock-in” transgenics of interest can be transgenic animals having a knock-in of the animal's endogenous BPntase. Such transgenics can be heterozygous knock-in for the BPntase gene, homozygous for the knock-in of the BPntase gene. “Knock-ins” also encompass conditional knock-ins, wherein the term “conditional” is used as defined above with respect to conditional knock-outs.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product, and “polypeptide” further includes “enzyme”, which is a polypeptide that acts as a biological catalyst. Therefore, polypeptide and all its iterations are used herein interchangeably with “enzyme.” In particular, enzymes of the sulfur assimilation pathway can be interchangeably referred to as polypeptides or enzymes to indicate the same molecule.

As used herein, the term “primer” means a sequence comprising two or more deoxyribonucleotides or ribonucleotides, preferably more than three, and more preferably more than eight and most preferably at least about 20 nucleotides of an exonic or intronic region. Such oligonucleotides are preferably between ten and thirty bases in length.

The term “subject” as used herein refers to any invertebrate or vertebrate species. The methods of the present disclosure are particularly useful in the treatment of warm-blooded vertebrates, e.g. in one embodiment, mammals and birds.

By “transgenic animal” is meant a non-human animal, usually a mammal (e.g., mouse, rat, rabbit, hamster, etc.), having a modified genome, for example, knocked out gene, knocked in gene, non-endogenous (i.e., heterologous) nucleic acid sequence present as an extrachromosomal element in a portion of its cells or stably integrated into its germline DNA (i.e., in the genomic sequence of most or all of its cells). A heterologous nucleic acid is introduced into the germ line of such transgenic animals by genetic manipulation of, for example, embryos or embryonic stem cells of the host animal. “Transgenic animal” as used herein, includes knock-outs and knock-ins.

The phrase “treating a neurological disorder”, for example, bipolar disorder or Alzheimer's disease, is meant to refer to the treatment of neurological disorders at any stage of progression. Thus, treatment of early onset neurological disorders as well as treatment of advanced neurological disorders falls within the phrase “treating a neurological disorder”. Preventing a neurological disorder and/or reducing the severity of a neurological disorder also fall within the phrase “treating a neurological disorder.”

II. Enzymes

In certain embodiments, methods (e.g., assays) for identifying and testing compounds that treat or mitigate lithium toxicity are provided. It has determined herein that sulfur assimilation enzyme pathways are relevant to lithium treatment, in regard to both therapeutic effects and undesirable toxic effects. Identifying compounds that interact with enzymes of these pathways is accordingly desirable, as such compounds can find use as alternative therapies to lithium, or as lithium antidotes to counteract the toxic effects of lithium.

Particularly useful are compounds that interact with (e.g., bind to, directly or indirectly regulate, directly or indirectly modulate, etc.) enzymes of the sulfur assimilation pathway. Sulfur assimilation pathway enzymes include, but are not limited to, ATP sulfurylases, APS kinases, sulfotransferases, PAPS reductases, and PAP phosphatases. Suitable ATP sulfurylase enzymes include but are not limited to, Met3 enzymes. Suitable APS kinase enzymes include but are not limited to, Met14 enzymes.

In a particular embodiment, compounds that interact with enzymes known as PAP phosphatases are identified. Compounds that interact with the bis-phosphate nucleotidase activity (BPntase), a PAP phosphatase, are particularly useful.

In certain embodiments, compounds that interact with (e.g., bind to, directly or indirectly regulate, directly or indirectly modulate, etc.) enzymes comprising the “lithium-sensitive family” (also referred to herein as the “lithium-sensitive phosphomonoesterase family”) of enzymes are useful. Known members of the lithium sensitive family of enzymes include, but are not limited to, fructose 1,6-bisphosphatase (fbptase), inositol monophosphatase (impase), inositol polyphosphatase (1ptase), HAL2(MET22), SAL1, cysQ, LPM Qa-X, suhB, MJ0109 and Bpntase (SEQ ID NO: 2).

In a certain embodiment, lithium-sensitive family enzymes comprise a metal binding site having the common sequence motif (SEQ ID NO: 3):
D-Xₙ-EE-Xₙ-DPiDgt-Xₙ-wd-X₁₁-GG,
   wherein:
X is any amino acid;
n is any integer;
D is aspartic acid;
E is glutamic acid;
P is proline;
G is glycine;
i is isoleucine or an amino acid that can be conservatively substituted in place thereof;
g is glycine or an amino acid that can be conservatively substituted in place thereof;
t is threonine or an amino acid that can be conservatively substituted in place thereof;
w is tryptophan or an amino acid that can be conservatively substituted in place thereof;
d is aspartic acid or an amino acid that can be conservatively substituted in place thereof.

This common motif is referred to herein interchangeably as the “unifying sequence motif” or the “lithium-sensitive motif.”

Although known sulfur assimilation pathway enzymes and PAP phosphatase polypeptides can be used in the foregoing methods, methods are also provided herein for the identification of heretofore unidentified members of these enzyme classes using the lithium-sensitive motif set forth above. Accordingly, methods for identifying a lithium sensitive family member protein are provided herein. These methods comprise screening a genetic database for a nucleotide sequence encoding a polypeptide homologous to the unifying sequence motif conserved within the family of lithium-sensitive family member proteins. This method was used in conjunction with EST databases to identify the mammalian family member, BPntase (Spiegelberg, 1999). The unifying sequence motif thus can be used to find other novel family members, using databases such as known and commercially available EST and genome databases.

A novel lithium-sensitive enzyme has been discovered using the methods of screening databases with the unifying sequence motif described herein, and this enzyme is useful in the practice of the screening methods described herein. The novel enzyme, believed to be an alternative splicing isoform of BPntase, has been named “LPM” for lithium-sensitive phosphomonoesterase. Northern blots of mRNA from multiple human tissues revealed that the message is expressed ubiquitously. ESTs encoding this enzyme have been discovered (for example, IMAGE clone 3855962, GenBank accession number BE962510), and cDNAs have been isolated from human libraries. The putative enzyme has been categorized as an isoform of Impase (GenBank accession number AY032885, Parthasarathy, 2001, SEQ ID NO: 1), but such an assignment has no published biochemical support.

Upon identifying an enzyme as a member of the lithium-sensitive family of enzymes, the artisan can optionally elect to demonstrate that the enzyme is in an active form. Such demonstrations can comprise illustrating that the putative family enzyme is able to bind Mg²⁺ with a K_{d} near 3 mM, consistent with other members of the lithium-sensitive family. Terbium fluorescence has been used to quantify Mg²⁺ binding to Impase (Pollack, 1993). In the active site of the enzyme, Tb³⁺ fluoresces following transfer of excitation energy from the active site tryptophan (Lin, 1991). When Mg²⁺ is titrated in, energy emission is reduced as Tb³⁺ is excluded from the active site (Lin, 1991). Such an assay would show that the putative enzyme is capable of binding Mg²⁺, and thus is appropriate for substrate analysis.

Enzymes of the present invention can be isolated or obtained from any organism, including but not limited to bacteria, archaebacteria, yeast, and mammals (e.g., rodents, primates, and humans). In certain embodiments, yeast sulfur assimilation pathway enzymes and/or lithium-sensitive family enzymes are used. In other embodiments, mouse sulfur assimilation pathway enzymes and/or lithium-sensitive family enzymes are used. In still other embodiments, human sulfur assimilation pathway enzymes and/or lithium-sensitive family enzymes are used.

III. Identifying Compounds Useful as Alternative Lithium Therapies and/or for Treating Lithium Toxicity:

Binding Assays

Methods for identifying compounds that modulate the activity of a sulfur assimilation pathway enzyme are provided in one embodiment. These methods generally comprise contacting a compound with a sulfur assimilation pathway enzyme and detecting modulation of the activity of the sulfur assimilation pathway enzyme. In particular embodiments, the sulfur assimilation pathway enzyme is selected from the group consisting of ATP sulfurylase, APS kinase, sulfotransferase, PAPS reductase, PAP phosphatase and combinations thereof. In certain embodiments, an ATP sulfurylase enzyme and an APS kinase enzyme together are form a bifunctional PAPS synthetase enzyme. The sulfur assimilation pathway enzyme can be selected from any organism. For example, although in some embodiments the sulfur assimilation pathway enzyme is a yeast sulfur assimilation pathway enzyme, and in other embodiments the sulfur assimilation pathway enzyme is a mammalian sulfur assimilation pathway enzyme.

Detecting modulation of the activity of the sulfur assimilation pathway enzyme can comprise detecting the binding of a compound to the sulfur assimilation pathway enzyme. In certain embodiments, detecting modulation of the activity of the sulfur assimilation pathway enzyme can comprise detecting inhibition of the activity of the sulfur assimilation pathway enzyme.

In particular embodiments, detecting modulation of the activity of the sulfur assimilation pathway enzyme comprises detecting a change in the amount of a sulfur assimilation pathway enzyme product. Suitable sulfur assimilation pathway products include, but are not limited to, APS, PAPS, PAP, AMP, cAMP, and combinations thereof.

In alternative embodiments, detecting modulation of the activity of the sulfur assimilation pathway enzyme comprises detecting a change in the amount of a sulfur assimilation pathway enzyme substrate. Suitable sulfur assimilation pathway substrates include but are not limited to ATP, APS, PAPS, PAP, AMP, and combinations thereof

A novel isotope binding competition assay, the GST-PST assay, was also developed to measure intracellular concentrations of PAP to further facilitate detecting modulation of PAP concentration.

Methods for identifying compounds that modulate the activity of a PAP phosphatase enzyme are also provided. These methods comprise contacting a compound with a PAP phosphatase polypeptide, and detecting modulation of the activity of the PAP phosphatase polypeptide. In one embodiment, a PAP phosphatase-modulating compound is selected if the compound modulates the activity of the PAP phosphatase polypeptide. In a particular embodiment, the PAP phosphatase is a BPntase. In a more particular embodiment, the PAP phosphatase is a mammalian BPntase.

In particular embodiments, detecting modulation of the activity of the PAP phosphatase polypeptide comprises detecting binding of the compound to the PAP phosphatase polypeptide. In certain embodiments, the compound binds at an active site of the PAP phosphatase polypeptide. Active sites include, but are not limited to, lithium binding sites and low affinity Mg²⁺ binding sites.

One particular embodiment can be used to identify compounds that modulate BPntase activity. The embodiment takes advantage of recombinant yeast genetic techniques and the properties of the gene encoding HAL2p/MET22p. Like lithium treatment, deletion of HAL2 causes a large increase in the intracellular concentration of PAP. hal2Δ yeast are also known to be auxotrophic for methionine. Conservation of BPntase function and the sulfur assimilation pathway, along with numerous other gene products, from yeast to man, means that results obtained in yeast are generally applicable to higher organisms.

Using the foregoing attributes, one type of genetic screen that can be used to identify compounds modulating BPntase or its substrates or products is an overexpression screen. In this screen, one searches for genes that when overexpressed complement a defect caused by a mutation in the gene of interest. In the case of BPntase, a library in which all known yeast genes are expressed at high levels is transformed into a strain of yeast in which the HAL2 gene has been deleted. The transformants are plated on media lacking methionine. Colonies that grow under these conditions are isolated as potential positives, and the gene products expressed on the plasmids contained in these yeast are identified as possible targets of PAP.

Growth in media lacking methionine causes the sulfur assimilation pathway to progress, and the lack of endogenous nucleotidase activity would causes the intracellular concentration of PAP to increase. One class of clones that complements this defect is the HAL2 gene itself. The activities of the gene product are (1) essential for the viability of the yeast under the conditions used and (2) inhibited by the accumulated PAP. Overexpression compensates for the inhibition by increasing the effective activity in the presence of the inhibitor.

The foregoing considerations were used to design a strategy to examine compounds for BPntase modulation activity. The strategy makes use of the ability of human BPntase to complement the methionine auxotrophy of yeast (e.g., S. cerevisiae) strains in which the HAL2 gene has been deleted. In one embodiment, hal2::Hal2p or ha/2::hBPntase yeast are inoculated into multi-well plates to assay for the inhibitory activity of a library of compounds. Each of the wells has minimal media lacking methionine (met⁻) and containing one of the potential modulating compounds to be evaluated. Following incubation at 30° C. for several days, the growth in each well is quantified, for example, by measuring the optical density.

Three results can be obtained, as follows: (1) if maximal growth is exhibited, the test compound probably has little effect on any essential activity; (2) if little growth by strains complemented with Hal2p or hBPntase is exhibited, the test compounds either inhibit some essential enzyme activity or inhibit both Hal2p and hBPntase, reverting them to methionine auxotrophy; (3) if maximal growth by strains complemented with Hal2p but little growth by strains complemented by hBPntase is exhibited, these compounds can inhibit hBPntase but have little effect on Hal2p. In this ideal situation, specificity of the compound is virtually assured, since it inhibits hBPntase but not an enzyme with a similar core structure and substrate specificity. The compound so identified can optionally be subjected to further study to determine a Kᵢ for inhibition of BPntase and other members of the lithium sensitive family. Synthetic chemistry can be employed to modify the compound to attempt to make it more potent or more specific.

Methods for identifying compounds that modulate the activity of a PAP phosphatase enzyme can thus comprise contacting a putative compound with the PAP phosphatase polypeptide by growing at least one recombinant yeast strain expressing the PAP phosphatase polypeptide in a minimal media lacking methionine and containing the compound, such that the compound contacts the recombinant yeast. In certain embodiments, the recombinant yeast strain is selected from the group consisting of hal2::Hal2p, hal2::BPntase, and combinations thereof. In more particular embodiments, the hal2::BPntase strain is a hal2::hBPntase (human BPntase).

As set forth above, in methods utilizing recombinant yeast strains, detecting modulation of the activity of the PAP phosphatase polypeptide can comprise measuring for growth of the recombinant yeast strain. Such growth measurement can comprise measuring a change in optical density. Compounds thus screened for PAP phosphatase modulating activity can be selected if growth of the recombinant yeast strain is inhibited. In a particular embodiment, the yeast strain is a combination of the hal2::Hal2p strain and the hal2::BPntase strain grown concurrently in minimal media lacking methionine and containing the compound and the compound is selected as the PAP phosphatase modulating compound if at least growth of the hal2::BPntase yeast strain is inhibited.

In the foregoing yeast-based assays, the growth conditions can optionally be manipulated by the artisan, for example, by using concentrations of methionine that are capable of reducing PAP levels but are not capable of supporting growth of hal2Δ yeast would increase the sensitivity of the assay. In addition, supplementation of the media with activated forms of sulfur, such as sulfite or sulfide, would make enzymes in the sulfur assimilation pathway dispensable and might then uncover complementation by overexpression of other activities, if such an inquiry is desired.

The known compound chlorate was screened using the recombinant yeast screening methods described herein, and ultimately identified as a compound that indirectly modulates BPntase, modulates production of PAP in vivo and lowers the toxicity of lithium. Chlorate was previously found to inhibit both yeast and mammalian ATP sulfurylase activities (Baeuerle, 1986; Foster, 1994; Ullrich, 2001b).

IV. Identifying Compounds Useful as Alternative Lithium Therapies and/or for Treating Lithium Toxicity:

Biochemical Assays

The potent toxicity of PAP to numerous enzymes and the detrimental effects of PAP accumulation in yeast led to the proposal that processes that decrease the intracellular concentrations of PAP can act as lithium antidotes. Such a lithium antidote could help to limit some of the numerous side effects related to lithium therapy.

As set forth above, one effect of BPntase inhibition is the accumulation of the substrate PAP. However, PAP targets might not be limited to those already described. PAP might interact with other enzymes that utilize nucleotides and might thus be a compound that represents general toxicity to cellular systems. Thus, the identification of compounds that interact with PAP is an aspect of this invention.

A biochemical approach can be used as a complementary technique to search for enzymes that interact with PAP. In a representative embodiment of this approach, extracts from organs or cell lines are passed over PAP-agarose resin. Proteins that are eluted from the column with free PAP likely bind this molecule, and biochemical assays are used to determine what effect PAP binding has on their in vitro activities.

V. Identifying Compounds Useful as Alternative Lithium Therapies and/or for Treating Lithium Toxicity:

Transgenic Models

Transgenic animals in which a gene encoding BPntase is knocked out are an aspect of the present disclosure. The generation of transgenic knockout animals facilitates the analysis of specific inhibition of BPntase in a whole organism, and provide animal models in which to test therapeutic agents that have the potential to mediate effects of lithium. The transgenic animals thus provide models of lithium-induced toxicity and methods of using the transgenic animals for identifying compounds that ameliorate lithium toxicity. The knock in animals provide models of overexpression of BPntase or expression of BPntase from other species, such as humans (SEQ ID NOs: 1 and 2), in the transgenic animals.

Accordingly, transgenic non-human vertebrate animals, having incorporated into their genomes a modified gene encoding a BPntase polypeptide (e.g. SEQ ID NO: 2), are provided herein. In certain embodiments, the modified gene encodes a biologically active human BPntase polypeptide. Modified genes can be incorporated into the genome so as to confer overexpression in the animal of the biologically active human BPntase polypeptide. Alternatively, the modified gene can be disrupted wherein the disrupted modified gene results in one of expression of a nonfunctional BPntase polypeptide and substantially no expression of a BPntase polypeptide. In certain embodiments, the disruption of the gene is a homozygous disruption.

Transgene Construction

The term “transgene” is used herein to describe genetic material that has been or is about to be artificially inserted into the genome of a mammalian cell, particularly a mammalian cell of a living animal. The transgene is used to transform a cell, meaning that a permanent or transient genetic change, preferably a permanent genetic change, is induced in a cell following incorporation of exogenous DNA. A permanent genetic change is generally achieved by introduction of the DNA into the genome of the cell.

Vectors for stable integration of the transgene include plasmids, retroviruses and other animal viruses, bacterial artificial chromosomes (BACs), yeast artificial chromosomes (YACs), cosmids and the like. The term “vector”, as used herein refers to a DNA molecule having sequences that enable its replication in a compatible host cell. A vector also includes nucleotide sequences to permit ligation of nucleotide sequences within the vector, wherein such nucleotide sequences are also replicated in a compatible host cell. A vector can also mediate recombinant production of a Bpntase polypeptide, as described further herein below. Representative vectors include but are not limited to those disclosed in Example 5.

Useful animals should be warm-blooded non-human vertebrates, for instance, mammals and birds. More particularly, the animal can be selected from the group consisting of rodent, swine, bird, ruminant, and primate. Even more particularly, the animal can be selected from the group consisting of a mouse, a rat, a pig, a guinea pig, poultry, an emu, an ostrich, a goat, a cow, a sheep, and a rabbit. Of interest are transgenic mammals, e.g. cows, pigs, goats, horses, etc., and particularly rodents, e.g. rats, mice, etc. Preferably, the transgenic animals are mice.

Transgenic animals comprise an exogenous nucleic acid sequence present as an extrachromosomal element or stably integrated in all or a portion of its cells, especially in germ cells. Unless otherwise indicated, it will be assumed that a transgenic animal comprises stable changes to the germline sequence. During the initial construction of the animal, “chimeras” or “chimeric animals” are generated, in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

The exogenous gene is usually either from a different species than the animal host, or is otherwise altered in its coding or non-coding sequence. The introduced gene can be a wild-type gene, naturally occurring polymorphism, or a genetically manipulated sequence, for example having deletions, substitutions or insertions in the coding or non-coding regions and can further include a marker gene for selection of cells transformed with the transgene. Where the introduced gene is a coding sequence, it is usually operably linked to a promoter, which can be constitutive or inducible, and other regulatory sequences required for expression in the host animal. By “operably linked” is meant that a DNA sequence and a regulatory sequence(s) are connected in such a way as to permit gene expression when the appropriate molecules, e.g. transcriptional activator proteins, are bound to the regulatory sequence(s).

In general, the transgenic animals disclosed herein comprise genetic alterations to provide for expression of a biologically active or inactive BPntase peptide. Preferably, the introduced sequences provide for alteration of the host's genome so as to affect the expression and function of endogenous genes (e.g., endogenous BPntase gene), contain marker genes, or other genetic alterations consistent with the goals disclosed herein. In particular, the introduced sequences provide for decreased, or even more preferably, no significant expression of the native BPntase gene, so as to result in onset of a medical condition similar to that observed in patient's suffering from lithium toxicity. Alternatively, the introduced sequences can result in high expression of BPntase so that overexpression of the BPntase gene is conferred in the transgenic animal. That is the transgene provides for increased levels of BPntase production relative to wild-type. The overexpressed BPntase can be either native BPntase, or a BPntase from a different species, for example, human BPntase.

Knockouts and Knockins

Although not necessary to the operability of the presently disclosed subject matter, the transgenic animals described herein can also comprise alterations to endogenous genes in addition to (or alternatively for BPntase), to the genetic alterations described above. For example, the host animals can be either “knock outs” and/or “knock ins” for a target gene(s) as is consistent with the goals of the invention (e.g., the host animal's endogenous BPntase can be “knocked out” and/or the endogenous BPntase gene “knocked in”). Knock outs have a partial or complete loss of function in one or both alleles of an endogenous gene of interest (e.g., BPntase). Knock ins have an introduced transgene with altered genetic sequence and/or function from the endogenous gene. The two can be combined, for example, such that the naturally occurring gene is disabled, and an altered form introduced. For example, it can be desirable to knockout the host animal's endogenous BPntase gene, while introducing an exogenous BPntase gene (e.g., a human BPntase gene).

In a knock out, preferably the target gene expression is undetectable or insignificant. For example, a knockout of a BPntase gene means that function of the BPntase has been substantially decreased so that expression is not detectable or only present at insignificant levels. This can be achieved by a variety of mechanisms, including introduction of a disruption of the coding sequence, e.g. insertion of one or more stop codons, insertion of a DNA fragment, etc., deletion of coding sequence, substitution of stop codons for coding sequence, etc. In some cases the exogenous transgene sequences are ultimately deleted from the genome, leaving a net change to the native sequence. Different approaches can also be used to achieve the “knock out”. A chromosomal deletion of all or part of the native gene can be induced, including deletions of the non-coding regions, particularly the promoter region, 3′ regulatory sequences, enhancers, or deletions of gene that activate expression of BPntase genes. A functional knock out can also be achieved by the introduction of an anti-sense construct that blocks expression of the native genes (for example, see Li and Cohen, 1996). “Knock outs” also include conditional knock-outs, for example where alteration of the target gene occurs upon exposure of the animal to a substance that promotes target gene alteration, introduction of an enzyme that promotes recombination at the target gene site (e.g. Cre in the Cre-lox system), or other method for directing the target gene alteration postnatally.

A “knock in” of a target gene means an alteration in a host cell genome that results in altered expression or function of a native target gene. Increased (including ectopic) or decreased expression can be achieved by introduction of an additional copy of the target gene, or by operatively inserting a regulatory sequence that provides for enhanced expression of an endogenous copy of the target gene. These changes can be constitutive or conditional, i.e. dependent on the presence of an activator or repressor. The use of knock in technology can be combined with production of exogenous sequences to produce the transgenic animals of the invention. For example, the BPntase transgenic animals of the invention can contain a knock in of the host's endogenous BPntase-encoding sequences to provide for the desired level of BPntase expression, and can contain an exogenous BPntase-encoding sequence.

Nucleic Acid Compositions

Constructs for use in the present invention include any construct suitable for use in the generation of transgenic animals having the desired levels of expression of a desired BPntase-encoding sequence. Methods for isolating and cloning a desired sequence, as well as suitable constructs for expression of a selected sequence in a host animal, are well known in the art. The construct can include sequences other than the BPntase-encoding sequences. For example, a detectable marker, such as lac Z can be included in the construct, where upregulation of expression of the encoded sequence will result in an easily detected change in phenotype.

The BPntase-encoding construct can contain a wild-type sequence encoding BPntase or a mutant sequence encoding BPntase. Likewise, the BPntase-encoding construct can contain a wild-type BPntase-encoding sequence or a sequence encoding a modified BPntase, particularly where the modification provides for a desired level of BPntase expression.

The term “BPntase gene” is used generically to mean BPntase genes, e.g. homologs from rat, human (SEQ ID NO: 1), mouse, guinea pig, etc., and their alternate forms. “BPntase gene” is also intended to mean the open reading frame encoding specific polypeptides, introns, and adjacent 5′ and 3′ non-coding nucleotide sequences involved in the regulation of expression, up to about 1 kb beyond the coding region, but possibly further in either direction. The DNA sequences encoding BPntase can be cDNA or genomic DNA or a fragment thereof. The genes can be introduced into an appropriate vector for extrachromosomal maintenance or for integration into the host.

The genomic sequences of particular interest comprise the nucleic acid present between the initiation codon and the stop codon, including all of the introns that are normally present in a native chromosome. They can further include the 3′ and 5′ untranslated regions found in the mature mRNA. They can further include specific transcriptional and translational regulatory sequences, such as promoters, enhancers, etc., including about 1 kb, but possibly more, of flanking genomic DNA at either the 5′ or 3′ end of the transcribed region. The genomic DNA can be isolated as a fragment of 100 kb or smaller; and substantially free of flanking chromosomal sequence.

The sequences of the 5′ regions of the BPntase gene, and further 5′ upstream sequences and 3′ downstream sequences, can be utilized for promoter elements, including enhancer-binding sites, which provide for expression in tissues where BPntase is normally expressed. The tissue specific expression is useful for providing promoters that mimic the native pattern of expression. Naturally occurring polymorphisms in the promoter region are useful for determining natural variations in expression, particularly those that can be associated with disease.

Alternatively, mutations can be introduced into the promoter region to determine the effect of altering expression in experimentally defined systems. Methods for the identification of specific DNA motifs involved in the binding of transcriptional factors are known in the art, e.g. sequence similarity to known binding motifs, gel retardation studies, etc. For examples, see Blackwell, 1995; Mortlock, 1996; and Joulin and Richard-Foy, 1995.

The nucleic acid compositions used in the subject invention can encode all or a part of BPntase as appropriate. Fragments can be obtained of the DNA sequence by chemically synthesizing oligonucleotides in accordance with conventional methods, by restriction enzyme digestion, by PCR amplification, etc. For the most part, DNA fragments will be of at least 15 nt, usually at least 18 nt, more usually at least about 50 nt. Such small DNA fragments are useful as primers for PCR, hybridization screening, etc. Larger DNA fragments, i.e. greater than 100 nt are useful for production of the encoded polypeptide. For use in amplification reactions, such as PCR, a pair of primers will be used.

Several isoforms and homologs of BPntase have been isolated and cloned. Additional homologs of cloned BPntase and/or BPntase are identified by various methods known in the art. Nucleic acids having sequence similarity are detected by hybridization under low stringency conditions, for example, at 50° C. and 10×SSC (0.9 M saline/0.09 M sodium citrate) and remain bound when subjected to washing at 55° C. in 1×SSC. Sequence identity can be determined by hybridization under more stringent conditions, for example, at 50° C. or higher and 0.1×SSC (9 mM saline/0.9 mM sodium citrate). By using probes, particularly labeled probes of DNA sequences, one can isolate homologous or related genes. The source of homologous genes can be any species, e.g. primate, rodents, canines, felines, bovines, ovines, equines, etc.

Where desirable, the BPntase sequences, including flanking promoter regions and coding regions, can be mutated in various ways known in the art to generate targeted changes in the sequence of the encoded protein, splice variant production, etc. The sequence changes can be substitutions, insertions or deletions. Deletions can include large changes, such as deletions of a domain or exon. Other modifications of interest include epitope tagging, e.g. with the FLAG system, HA, etc. For studies of subcellular localization, fusion proteins with green fluorescent proteins (GFP) can be used. Such mutated genes can be used to study structure-function relationships of BPntase, or to alter properties of the proteins that affect their function or regulation. The BPntase encoding sequence can also be provided as a fusion protein. Methods for production of BPntase constructs are well known in the art (see, e.g., Wyss-Coray, 1995).

Techniques for in vitro mutagenesis of cloned genes are known. Examples of protocols for scanning mutations can be found in Gustin, 1993; Barany, 1985; Colicelli, 1985; and Prentid, 1984. Methods for site specific mutagenesis can be found in Sambrook, 1989; Weiner, 1993; Sayers, 1992; Jones and Winistorfer, 1992; Barton, 1990; Marotti and Tomich, 1989; and Zhu, 1989.

The BPntase gene, and exemplary derivatives thereof suitable for use in the production of the transgenic animals of the invention can be either genomic or cDNA, preferably cDNA, and can be derived from any source, e.g., human, murine, porcine, bovine, etc. Several BPntase sequences have been isolated, cloned, and sequenced (see e.g. SEQ ID NO: 1).

The host animals can be homozygous or heterozygous for the BPntase-encoding sequence, preferably homozygous. The BPntase gene can also be operably linked to a promoter to provide for a desired level of expression in the host animal and/or for tissue-specific expression. Expression of BPntase can be either constitutive or inducible, preferably constitutive.

Indeed, in general terms, one embodiment of the transgene was prepared in the following manner.

Methods of Making BPntase Transgenic Animals

Disclosed herein is a method of preparing a transgenic non-human animal that expresses either a functional or non-functional BPntase gene. A preferred transgenic animal is a mouse.

Techniques for the preparation of transgenic animals are known in the art. Example 5 sets forth one example of the techniques useful for generating transgenic animals. Other exemplary techniques are described in U.S. Pat. No. 5,489,742 (transgenic rats); U.S. Pat. Nos. 4,736,866, 5,550,316, 5,614,396, 5,625,125 and 5,648,061 (transgenic mice); U.S. Pat. No. 5,573,933 (transgenic pigs); U.S. Pat. No. 5,162,215 (transgenic avian species) and U.S. Pat. No. 5,741,957 (transgenic bovine species), the entire contents of each of which are herein incorporated by reference.

With respect to a representative method for the preparation of a transgenic mouse, cloned recombinant or synthetic DNA sequences or DNA segments encoding a BPntase gene product are injected into fertilized mouse eggs. The injected eggs are implanted in pseudo pregnant females and are grown to term to provide transgenic mice whose cells express a BPntase gene product.

DNA constructs for random integration need not include regions of homology to mediate recombination. Where homologous recombination is desired, the DNA constructs will comprise at least a portion of the target gene with the desired genetic modification, and will include regions of homology to the target locus. Conveniently, markers for positive and negative selection are included. Methods for generating cells having targeted gene modifications through homologous recombination are known in the art. For various techniques for transfecting mammalian cells, see Keown, 1990.

For embryonic stem (ES) cells, an ES cell line can be employed, or embryonic cells can be obtained freshly from a host, e.g. mouse, rat, guinea pig, etc. Such cells are grown on an appropriate fibroblast-feeder layer or grown in the presence of appropriate growth factors, such as leukemia inhibiting factor (LIF). When ES cells have been transformed, they can be used to produce transgenic animals. After transformation, the cells are plated onto a feeder layer in an appropriate medium. Cells containing the construct can be detected by employing a selective medium. After sufficient time for colonies to grow, they are picked and analyzed for the occurrence of homologous recombination or integration of the construct. Those colonies that are positive can then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting litters screened for mutant cells having the construct. By providing for a different phenotype of the blastocyst and the ES cells, chimeric progeny can be readily detected.

The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture.

A transgenic animal, as disclosed herein, preferably comprises a mouse with targeted modification of the BPntase gene. Mice strains with complete or partial functional inactivation of the BPntase gene in all somatic cells are generated using standard techniques of site-specific recombination in murine embryonic stem cells. See Capecchi, 1989 and Thomas & Capecchi, 1990.

The present invention also provides mice strains with specific “knocked-in” modifications in the BPntase gene. This includes mice with genetically and functionally relevant point mutations in the BPntase gene, in addition to manipulations such as the insertion of specific repeat expansions.

In particularly desirable embodiments, expression of the BPntase polypeptide is specifically conferred in a tissue or blood of the transgenic animal. Tissues useful for such selective expression include but are not limited to kidney tissue, brain tissue, liver tissue, intestinal tissue, skin tissue, heart tissue, lung tissue, spleen tissue, bone marrow, and combinations thereof.

In one embodiment, the transgenic animal is a mouse. The mBPntase sequence, obtained from available mouse genome information, is used to create a transgenic mouse. The BPntase gene can be deleted using at least two methods. In one method, the gene is knocked out in embryonic stem cells that are implanted in parental mice. This strategy provides mouse strains in which BPntase is deleted in all cells at all stages of development. Considering the ubiquity of the enzyme's expression, this method could pose a problem in that defects in specific organs and tissues can be masked by a general toxicity of the deletion.

To overcome this potential difficulty, the BPntase gene can also be knocked out in a ‘conditional’ manner. In this case, the gene remains intact until the knockout is induced. To create this inducible system, loxP sites are introduced on both sides of a portion of the gene (for review see Sauer, 1998). The strain thus created is mated with previously created strains in which the Cre recombinase is expressed under promoters that are only activated in a specific subset of tissues or at a specific stage of development. Recombination catalyzed by Cre deletes the sequence between the loxP sites in the tissue of interest. The role of BPntase in a particular area or stage can then be investigated by studying the mouse's phenotype (the acquisition of NDI, for example) or by culturing cells from the organism and analyzing them biochemically.

An alternative method for using genetic information to specifically inhibit an enzyme's activity is through the use of RNA-mediated inhibition. This technique, which was initially identified in C. elegans (for review see Fire, 1999), has recently been translated to use in mammalian cell culture (Elbashir, 2001; Harborth, 2001; Hudson, 2002). In mammalian cells, synthetic 21 to 23 bp double stranded RNAs, called short interfering RNAs or siRNAs (Hudson, 2002) are transfected or micro-injected into cells. Through mechanisms that have yet to be clearly elucidated (Hudson, 2002), the mRNA containing the sequence of the siRNA is degraded, leading to a ‘knockdown’ of the expression of the protein of interest (Elbashir, 2001; Harborth, 2001; Hudson, 2002). This technique has been used successfully in a number of cases, but it has two main drawbacks. One is that it is dependent on the type of cell used. Theoretically, to see 100% reduction in enzyme activity, every cell must take up the dsRNA during the transfection. Another potential problem is that inhibition is dependent on relatively rapid turnover of the protein of interest (Fire, 1999). A protein with a long half-life within the cell might not be affected by deletion of its mRNA.

To overcome these difficulties, techniques for the stable expression of siRNA within the cell have been developed (Brummelkamp, 2002; Paul, 2002). In this case, cells that are subject to siRNA knockdown can be selected by co-transfecting the cells with the siRNA plasmid and a plasmid conferring antibiotic resistance. In addition, protein stability can be eliminated as a factor in the experiment since the cells can be cultured for long periods of time. Establishment of these stable cells lines provides useful reagents for the detection of the consequences of BPntase inhibition on function of systems such as sulfur assimilation, cAMP generation, and epithelial molecular transport. In addition, it provides a mammalian system with which to test compounds (e.g., chlorate mimetics) for their ability to complement defects caused by BPntase inhibition.

Knockdown of BPntase expression in a cell line phenocopies lithium treatment. Specifically, genetic disruption of BPntase activity also phenocopies lithium in this system as it does with the disruption of HAL2 in yeast. Specific inhibitors of mammalian BPntase phenocopies lithium's effect on the stimulation of adenylate cyclase in vivo. Likewise, chlorate mimetics phenocopy chlorate's rescue of lithium toxicity. Specifically, animals generated using the foregoing methods are expected to develop lithium-independent NDI.

Thus, transgenic animals as described herein are useful in methods of identifying a compound for treating a toxic effect resulting from a therapeutic treatment. In one embodiment, these methods comprise (a) obtaining a transgenic non-human vertebrate animal having incorporated into its genome a disruption of a gene encoding a BPntase polypeptide, wherein the disruption results in the transgenic animal exhibiting the toxic effect; (b) administering the compound to the transgenic animal; and (c) observing the transgenic animal for a change in the transgenic animal indicative of amelioration of the effect.

In particular embodiments, the therapeutic treatment is a lithium treatment for a neurological disorder. Neurological disorders include Alzheimer's disease and bipolar disorder.

In particular embodiments, toxic effects include kidney dysfunction, emesis, diarrhea, organ dysfunction, hypothyroidism, and combinations thereof.

VI. Identifying Compounds Useful as Alternative Lithium Therapies and/or for Treating Lithium Toxicity:

Computer Modeling

Structural and kinetic studies of Impase have been the main sources of information concerning the metal binding sites of lithium sensitive family members. The well-conserved residues of the lithium sensitivity motif have been shown through crystallographic analyses to be central to the binding of catalytic metals. Asp-47, Glu-70, Glu-71, Asp-90, the backbone oxygen of lie-92, Asp-93, and Asp 220 have been shown to coordinate divalent cations (Bone, 1994a; Bone, 1994b; Bone, 1992) (FIG. 2). In addition, while Thr-95 does not appear to bind metal directly (FIG. 2), it has been shown via mutagenesis experiments to be required for catalysis (Pollack, 1994; Pollack, 1993). This same data is equally relevant and correlates with the metal binding site of BPntase and therefore is useful for modeling the lithium binding pocket of BPntase.

The discovery of bisphosphate 3′-nucleotidase, or BPntase (SEQ ID NOs: 1 and 2), represents the first description of a mammalian bisphosphate 3′-nucleotidase activity, and was isolated using a “computer cloning” strategy, in which the lithium-sensitivity motif was used in conjunction with EST databases.

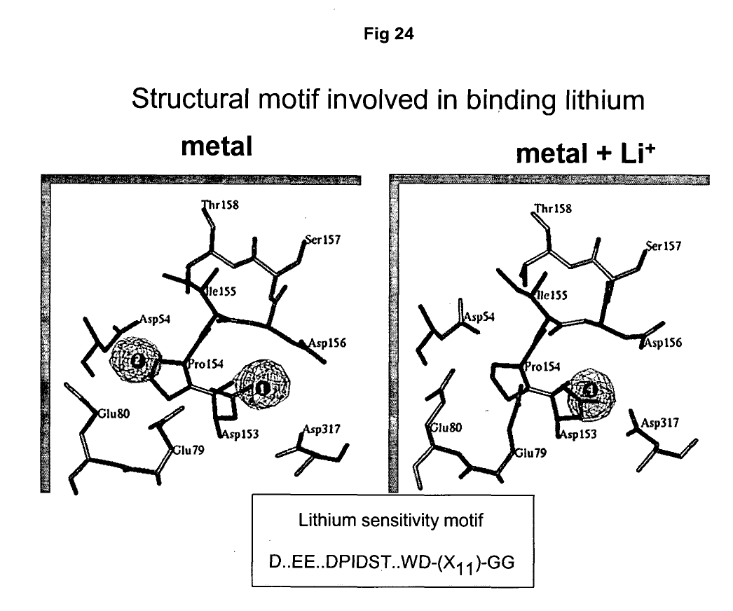

Crystallization and mutagenic experiments using techniques known to one of skill in the art have localized the site of lithium binding to the ‘DPIDST’ motif. By looking at difference density maps that showed the electron density obtained when the structure of 1ptase crystallized under various conditions, in combination with mutagenic experiments, a determination of the lithium binding site is made for polypeptides of the lithium binding family. FIG. 24 shows the lithium binding site in 1-phosphatase (1ptase). When 1ptase was crystallized in the presence of metal, electron density is seen at two sites, corresponding to the two metal binding sites (FIG. 24, left panel). This is confirmed by previous kinetic data. When 1ptase is crystallized in the presence of metal and lithium, however, the density at metal site 2 disappears (FIG. 24, right panel). This is explained by the fact that lithium, having only 2 electrons, is invisible in this experiment. Therefore, the data shows a loss of occupancy at metal site 2 by invisible lithium binding instead, excluding the binding of more electron rich metals. Mutagenic analysis of D54A (aspartate to alanine) confirms this hypothesis. With this mutation, the Ki for lithium changes from 0.5 M to 100 mM.

These data provide guidance in modeling interactions with the BPntase polypeptide. Data from these experiments elucidate the lithium binding site to a ‘DPIDST’ motif as well. As with the other lithium-sensitive family members, however, the data confirm that the ‘DPIDST’ motif alone is insufficient. An extended lithium sensitivity motif, in accord with other lithium-sensitive family members, has also been determined. The lithium sensitivity motif for Bpntase is now known to be Asp-51, Glu-74, Glu-75, Asp-117, Pro-118, Leu-119, Asp-120, Gly-121, Thr-122, and Asp-247. However, lithium interacts primarily with Asp-51, Glu-74, Asp-117, and Leu-119 and so these minimal residues are particularly favored as target sites for therapeutics.

Accordingly, additional embodiments of the invention include methods of modeling target sites for lithium, and methods of modeling related compounds on lithium sensitive molecules for identifying additional compounds capable of binding the target sites. Particularly, methods of identifying a compound that modulates the activity of a BPntase polypeptide comprise modeling an interaction between the compound and a target moiety on the BPntase polypeptide. In a particular embodiment, the modeling used is computer modeling. Interactions that can be modeled include but are not limited to binding of the compound to the BPntase polypeptide by hydrogen bonding, van der Waal's binding, or both hydrogen bonding and van der Waal's bonding.

In the modeling methods described herein, suitable target moieties include lithium binding sites and low affinity Mg²⁺ binding sites. A particular target moiety, or druggable region, is a cluster of amino acid residues fixed at specific spatial points as determined by secondary structure of the BPntase peptide and confirmed by x-ray crystallography, the residues being Asp-51, Glu-74, Glu-75, Asp-117, Pro-118, Leu-119, Asp-120, Gly-121, Thr-122, and Asp-247. More preferably, the residues are Asp-51, Glu-74, Asp-117, and Leu-119.

A number of techniques can be used to screen, identify, select, and design chemical entities capable of associating with polypeptides disclosed herein, structurally homologous molecules, and other molecules. Knowledge of the structure for a polypeptide disclosed herein, determined in accordance with the methods described herein, permits the design and/or identification of molecules and/or other modulators that have a shape complementary to the conformation of a polypeptide, or a portion of a polypeptide, disclosed herein, or more particularly, a druggable region thereof. As a non-limiting example, the low affinity Mg²⁺ binding site of BPntase can act as a target moiety for designing or identifying molecules that modulate the activity of BPntase. It is understood that such techniques and methods can use, in addition to the exact structural coordinates and other information for a polypeptide of the invention, structural equivalents thereof (including, for example, those structural coordinates that are derived from the structural coordinates of amino acids contained in a druggable region as described above).

The term “chemical entity”, as used herein, refers to chemical compounds, complexes of two or more chemical compounds, and fragments of such compounds or complexes. In certain instances, it is desirable to use chemical entities exhibiting a wide range of structural and functional diversity, such as compounds exhibiting different shapes (i.e., flat aromatic rings(s), puckered aliphatic rings(s), straight and branched chain aliphatics with single, double, or triple bonds) and diverse functional groups (i.e., carboxylic acids, esters, ethers, amines, aldehydes, ketones, and various heterocyclic rings).

In one aspect, the method of drug design generally includes computationally evaluating the potential of a selected chemical entity to associate with any of the molecules or complexes disclosed herein (or portions thereof). For example, this method can include the steps of (a) employing computational means to perform a fitting operation between the selected chemical entity and a druggable region of the molecule or complex; and (b) analyzing the results of said fitting operation to quantify the association between the chemical entity and the druggable region.

A chemical entity can be examined either through visual inspection or through the use of computer modeling using a docking program such as GRAM, DOCK, or AUTODOCK (Dunbrack, 1997). This procedure can include computer fitting of chemical entities to a target to ascertain how well the shape and the chemical structure of each chemical entity will complement or interfere with the structure of the subject polypeptide (Bugg, 1993; West, 1995). Computer programs can also be employed to estimate the attraction, repulsion, and steric hindrance of the chemical entity to a druggable region due to hydrogen binding, van der Waal's binding, etc., for example. Generally, the tighter the fit (i.e., the lower the steric hindrance, and/or the greater the attractive force) the more potent the chemical entity will be because these properties are consistent with a tighter binding constant. Furthermore, the more specificity in the design of a chemical entity the more likely that the chemical entity will not interfere with related proteins, which can minimize potential side-effects due to unwanted interactions.

A variety of computational methods for molecular design, in which the steric and electronic properties of druggable regions are used to guide the design of chemical entities, are known. See e.g., Cohen, 1990; Kuntz, 1982; DesJarlais, 1988; Bartlett, 1989; Goodford, 1985; DesJarlais, 1986. Directed methods generally fall into two categories: (1) design by analogy in which 3-D structures of known chemical entities (such as from a crystallographic database) are docked to the druggable region and scored for goodness-of-fit; and (2) de novo design, in which the chemical entity is constructed piece-wise in the druggable region. The chemical entity can be screened as part of a library or a database of molecules. Databases which can be used include ACD (MDL Systems Inc., San Leandro, Calif., United States of America), NCI (National Cancer Institute, Bethesda, Md., United States of America), CCDC (Cambridge Crystallographic Data Center, Cambridge, England, United Kingdom), CAST (Chemical Abstract Service), Derwent (Derwent Information Limited, London, England, United Kingdom), Maybridge (Maybridge Chemical Company Ltd., Cornwall, England, United Kingdom), Aldrich (Aldrich Chemical Company, St. Louis, Mo., United States of America), DOCK (University of California in San Francisco, San Francisco, Calif., United States of America), and the Directory of Natural Products (Chapman & Hall). Computer programs such as CONCORD (Tripos Inc., St. Louis, Mo., United States of America) or DB-Converter (Molecular Simulations Limited, Cambridge, England, United Kingdom) can be used to convert a data set represented in two dimensions to one represented in three dimensions.

Chemical entities can be tested for their capacity to fit spatially with a druggable region or other portion of a target protein. As used herein, the term “fits spatially” means that the three-dimensional structure of the chemical entity is accommodated geometrically by a druggable region. A favorable geometric fit occurs when the surface area of the chemical entity is in close proximity with the surface area of the druggable region without forming unfavorable interactions. A favorable complementary interaction occurs where the chemical entity interacts by hydrophobic, aromatic, ionic, dipolar, or hydrogen donating and accepting forces. Unfavorable interactions can be steric hindrance between atoms in the chemical entity and atoms in the druggable region.

If a model of disclosed herein is a computer model, the chemical entities can be positioned in a druggable region through computational docking. If, on the other hand, the model disclosed herein is a structural model, the chemical entities can be positioned in the druggable region by, for example, manual docking. As used herein the term “docking” refers to a process of placing a chemical entity in close proximity with a druggable region, or a process of finding low energy conformations of a chemical entity/druggable region complex.

In an illustrative embodiment, the design of potential modulator begins from the general perspective of shape complimentary for the druggable region of a polypeptide of the invention, and a search algorithm is employed which is capable of scanning a database of small molecules of known three-dimensional structure for chemical entities which fit geometrically with the target druggable region. Most algorithms of this type provide a method for finding a wide assortment of chemical entities that are complementary to the shape of a druggable region of the subject polypeptide. Each of a set of chemical entities from a particular data-base, such as the Cambridge Crystallographic Data Bank (CCDB) (Allen, 1973), is individually docked to the druggable region of a polypeptide of the invention in a number of geometrically permissible orientations with use of a docking algorithm. In certain embodiments, a set of computer algorithms called DOCK, can be used to characterize the shape of invaginations and grooves that form the active sites and recognition surfaces of the druggable region (Kuntz, 1982). The program can also search a database of small molecules for templates whose shapes are complementary to particular binding sites of a polypeptide of the invention (DesJarlais, 1988).

The orientations are evaluated for goodness-of-fit and the best are kept for further examination using molecular mechanics programs, such as AMBER or CHARMM. Such algorithms have previously proven successful in finding a variety of chemical entities that are complementary in shape to a druggable region.

Goodford, 1985 and Boobbyer, 1989 have produced a computer program (GRID) that seeks to determine regions of high affinity for different chemical groups (termed probes) of the druggable region. GRID hence provides a tool for suggesting modifications to known chemical entities that might enhance binding. It can be anticipated that some of the sites discerned by GRID as regions of high affinity correspond to “pharmacophoric patterns” determined inferentially from a series of known ligands. As used herein, a “pharmacophoric pattern” is a geometric arrangement of features of chemical entities that is believed to be important for binding. Attempts have been made to use pharmacophoric patterns as a search screen for novel ligands (Jakes, 1987; Brint & Willett, 1987; Jakes, 1986).

Yet a further embodiment of the present invention utilizes a computer algorithm such as CLIX which searches such databases as CCDB for chemical entities which can be oriented with the druggable region in a way that is both sterically acceptable and has a high likelihood of achieving favorable chemical interactions between the chemical entity and the surrounding amino acid residues. The method is based on characterizing the region in terms of an ensemble of favorable binding positions for different chemical groups and then searching for orientations of the chemical entities that cause maximum spatial coincidence of individual candidate chemical groups with members of the ensemble. The algorithmic details of CLIX are described in Lawrence, 1992.

In this way, the efficiency with which a chemical entity can bind to or interfere with a druggable region can be tested and optimized by computational evaluation. For example, for a favorable association with a druggable region, a chemical entity must preferably demonstrate a relatively small difference in energy between its bound and fine states (i.e., a small deformation energy of binding). Thus, certain, more desirable chemical entities will be designed with a deformation energy of binding of not greater than about 10 kcal/mole, and more preferably, not greater than 7 kcal/mole. Chemical entities can interact with a druggable region in more than one conformation that is similar in overall binding energy. In those cases, the deformation energy of binding is taken to be the difference between the energy of the free entity and the average energy of the conformations observed when the chemical entity binds to the target.

In this way, computer-assisted methods are provided for identifying or designing a potential modulator of the activity of a polypeptide disclosed herein including: supplying a computer modeling application with a set of structure coordinates of a molecule or complex, the molecule or complex including at least a portion of a druggable region from a polypeptide of the invention; supplying the computer modeling application with a set of structure coordinates of a chemical entity; and determining whether the chemical entity is expected to bind to the molecule or complex, wherein binding to the molecule or complex is indicative of potential modulation of the activity of a polypeptide of the invention.

In another aspect, provided is a computer-assisted method for identifying or designing a potential modulator to a polypeptide disclosed herein, for example BPntase, by supplying a computer modeling application with a set of structure coordinates of a molecule or complex, the molecule or complex including at least a portion of a druggable region of a polypeptide of the invention; supplying the computer modeling application with a set of structure coordinates for a chemical entity; evaluating the potential binding interactions between the chemical entity and active site of the molecule or molecular complex; structurally modifying the chemical entity to yield a set of structure coordinates for a modified chemical entity, and determining whether the modified chemical entity is expected to bind to the molecule or complex, wherein binding to the molecule or complex is indicative of potential modulation of the polypeptide of the invention.

In one embodiment, a potential modulator can be obtained by screening a peptide library (Scott & Smith, 1990; Cwirla, 1990; Devlin, 1990). A potential modulator selected in this manner could then be systematically modified by computer modeling programs until one or more promising potential drugs are identified. Such analysis has been shown to be effective in the development of HIV protease inhibitors (Lam, 1994; Wlodawer, 1993; Appelt, 1993; Erickson, 1993). Alternatively a potential modulator can be selected from a library of chemicals such as those that can be licensed from third parties, such as chemical and pharmaceutical companies. A third alternative is to synthesize the potential modulator de novo.

For example, in certain embodiments, provided is a method for making a potential modulator for a polypeptide disclosed herein, the method including synthesizing a chemical entity or a molecule containing the chemical entity to yield a potential modulator of a polypeptide disclosed herein, the chemical entity having been identified during a computer-assisted process including supplying a computer modeling application with a set of structure coordinates of a molecule or complex, the molecule or complex including at least one druggable region from a polypeptide disclosed herein; supplying the computer modeling application with a set of structure coordinates of a chemical entity; and determining whether the chemical entity is expected to bind to the molecule or complex at the active site, wherein binding to the molecule or complex is indicative of potential modulation. This method can further include the steps of evaluating the potential binding interactions between the chemical entity and the active site of the molecule or molecular complex and structurally modifying the chemical entity to yield a set of structure coordinates for a modified chemical entity, which steps can be repeated one or more times.

Once a potential modulator is identified, it can then be tested in any standard assay for the macromolecule depending of course on the macromolecule, including in high throughput assays. Further refinements to the structure of the modulator will generally be necessary and can be made by the successive iterations of any and/or all of the steps provided by the particular screening assay, in particular further structural analysis by i.e., 15N NMR relaxation rate determinations or X-ray crystallography with the modulator bound to the subject polypeptide. These studies can be performed in conjunction with biochemical assays.

Once identified, a potential modulator can be used as a model structure, and analogs to the compound can be obtained. The analogs are then screened for their ability to bind the subject polypeptide. An analog of the potential modulator might be chosen as a modulator when it binds to the subject polypeptide with a higher binding affinity than the predecessor modulator.

In a related approach, iterative drug design is used to identify modulators of a target protein. Iterative drug design is a method for optimizing associations between a protein and a modulator by determining and evaluating the three dimensional structures of successive sets of protein/modulator complexes. In iterative drug design, crystals of a series of protein/modulator complexes are obtained and then the three-dimensional structures of each complex is solved. Such an approach provides insight into the association between the proteins and modulators of each complex. For example, this approach can be accomplished by selecting modulators with inhibitory activity, obtaining crystals of this new protein/modulator complex, solving the three dimensional structure of the complex, and comparing the associations between the new protein/modulator complex and previously solved protein/modulator complexes. By observing how changes in the modulator affected the protein/modulator associations, these associations can be optimized.

In addition to designing and/or identifying a chemical entity to associate with a druggable region, as described above, the same techniques and methods can be used to design and/or identify chemical entities that either associate, or do not associate, with affinity regions, selectivity regions or undesired regions of protein targets. By such methods, selectivity for one or a few targets, or alternatively for multiple targets, from the same species or from multiple species, can be achieved.

For example, a chemical entity can be designed and/or identified for which the binding energy for one druggable region, i.e., an affinity region or selectivity region, such as a lithium-binding moiety, is more favorable than that for another region, i.e., an undesired region, by about 20%, 30%, 50% to about 60% or more. It can be the case that the difference is observed between (a) more than two regions, (b) between different regions (selectivity, affinity or undesirable) from the same target, (c) between regions of different targets, (d) between regions of homologs from different species, or (e) between other combinations. Alternatively, the comparison can be made by reference to the K_{d}, usually the apparent K_{d}, of said chemical entity with the two or more regions in question.

In another aspect, prospective modulators are screened for binding to two nearby druggable regions on a target protein. For example, a modulator that binds a first region of a target polypeptide does not bind a second nearby region. Binding to the second region can be determined by monitoring changes in a different set of amide chemical shifts in either the original screen or a second screen conducted in the presence of a modulator (or potential modulator) for the first region. From an analysis of the chemical shift changes, the approximate location of a potential modulator for the second region is identified. Optimization of the second modulator for binding to the region is then carried out by screening structurally related compounds (i.e., analogs as described above).

When modulators for the first region and the second region are identified, their location and orientation in the ternary complex can be determined experimentally. On the basis of this structural information, a linked compound, i.e., a consolidated modulator, is synthesized in which the modulator for the first region and the modulator for the second region are linked. In certain embodiments, the two modulators are covalently linked to form a consolidated modulator. This consolidated modulator can be tested to determine if it has a higher binding affinity for the target than either of the two individual modulators. A consolidated modulator is selected as a modulator when it has a higher binding affinity for the target than either of the two modulators. Larger consolidated modulators can be constructed in an analogous manner, i.e., linking three modulators which bind to three nearby regions on the target to form a multilinked consolidated modulator that has an even higher affinity for the target than the linked modulator. In this example, it is assumed that is desirable to have the modulator bind to all the druggable regions. However, it can be the case that binding to certain of the druggable regions is not desirable, so that the same techniques can be used to identify modulators and consolidated modulators that show increased specificity based on binding to at least one but not all druggable regions of a target.

Also provided is a method for identifying a potential inhibitor of a polypeptide disclosed herein, the method comprising: (a) providing the three-dimensional coordinates of a polypeptide disclosed herein or a fragment thereof; (b) identifying a druggable region of the polypeptide or fragment; and (c) selecting from a database at least one compound that comprises three dimensional coordinates which indicate that the compound can bind the druggable region; (d) wherein the selected compound is a potential inhibitor of a polypeptide of the invention.

Another aspect disclosed herein includes a method for designing a potential compound for the prevention or treatment of a disease or disorder, such as bipolar disorder or lithium toxicity related to lithium treatment, the method comprising: (a) providing the three dimensional structure of a polypeptide disclosed herein, such as an enzyme of the sulfur assimilation pathway, and BPntase in particular, or a fragment thereof; (b) synthesizing a potential compound for the prevention or treatment of a disease or disorder based on the three dimensional structure of the polypeptide or fragment; (c) contacting the polypeptide with the potential compound; and (d) assaying the activity of the polypeptide, wherein a change in the activity of the polypeptide indicates that the compound can be useful for prevention or treatment of a disease or disorder. Optionally, the polypeptide can be crystallized, and the three dimensional structure can be prepared from the crystallized polypeptide.

VII. Methods of Treating Lithium Toxicity

Compounds identified by the foregoing screening methods are useful as treatments for lithium induced toxicity. Compounds identified by the inventive methods are also useful as alternative therapeutics to lithium treatment.

Accordingly, methods for treating lithium-related toxicity are provided by the methods and assays and compounds described herein. These treatment methods generally comprise administering to a subject suffering from a lithium-related toxic effect a therapeutically effective amount of a compound that modulates the activity of at least one sulfur assimilation pathway enzyme.

Lithium-related toxic effects include but are not limited to nausea, emesis, diarrhea, organ dysfunction, hypothyroidism, and combinations thereof. Particular organ dysfunctions include but are not limited kidney dysfunction, brain dysfunction, and dysfunctions of the gastrointestinal organs.

Compounds useful in these methods can include those identified by the recombinant yeast-based assays described herein, such as chlorate and mimetics thereof. Compounds that inhibit PAPS synthetase activity are also potentially therapeutic adjuncts to limit the toxicity of lithium. These inhibitors can be screened searched for using an assay similar to the yeast-based assay for hBPntase inhibitors, as human PAPS synthetase has been cloned and has been shown to complement the methionine auxotrophy of met3Δ and met14Δ. Compounds that inhibit growth of both yeast strains described herein are of particular interest and are a further embodiment.

Considering the connection between BPntase and the therapeutic effects of lithium, a specific inhibitor of the enzyme is a candidate compound for the development of alternatives treatments for bipolar disorder. Specific inhibitors of PAPS synthetases can also be valuable as lithium antidotes, minimizing side effects of this potentially toxic drug.

Formulation preparation techniques have been generally described in the art, see for example, those described in U.S. Pat. No. 5,326,902 issued to Seipp et al. on Jul. 5, 1994, U.S. Pat. No. 5,234,933 issued to Marnett et al. on Aug. 10, 1993, and PCT Publication WO 93/25521 of Johnson et al. published Dec. 23, 1993, and each of which is herein incorporated by reference in its entirety.

For the purposes described above, the therapeutic agent, such as a compound that modulates an enzyme of the sulfur assimilation pathway, and including BPntase, can normally be administered systemically or partially, usually by oral or parenteral administration. The doses to be administered are determined depending upon age, body weight, symptom, the desired effect, the route of administration, and the duration of the treatment, etc. One of skill in the art of therapeutic treatment will recognize appropriate procedures and techniques for determining the appropriate dosage regimen for effective therapy. Various compositions and forms of administration are provided and are generally known in the art. Other compositions for administration include suppositories that comprise one or more of the active substance(s) and can be prepared by known methods.

Thus, a pharmaceutical composition in accordance with the present invention can be formulated with one or more physiologically acceptable carriers or excipients. Thus, the compounds for use according to the present invention can be formulated for oral, buccal, sublingual, parenteral, rectal or transdermal administration, or administration in a form suitable for inhalation or insufflation (either through the mouth or the nose). In one embodiment a transdermal patch is employed. In another embodiment an oral preparation is employed. In another embodiment, an injection that has long term benefits is employed, e.g. a sustained release formulation. Administration can also be accomplished by any other effective techniques.

For oral administration, the pharmaceutical compositions can take the form of, for example, tablets or capsules prepared by a conventional technique with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). The tablets can be coated by methods well known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The methods of administration according to the present invention can include parenteral administration by injection, for example by bolus injection or continuous infusion. Formulations for injection can be presented in unit dosage form, e.g., in ampoules or in multi-dose containers, with or without an added preservative. An injectable formulation can be used in delivering a therapeutic agent across the blood brain barrier to the central nervous system.

The compositions used in the methods can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g. sterile pyrogen-free water, before use. The compounds can also be formulated in rectal compositions such as suppositories or retention enemas, e.g. containing conventional suppository bases such as cocoa butter or other glycerides. In addition to the formulations described previously, the compounds can also be formulated as a preparation for implantation or injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

### EXAMPLES

The presently disclosed subject matter will be further disclosed by reference to the following detailed examples. These examples are provided for purposes of illustration only, and are not intended to be limiting unless otherwise specified.

### Example 1

### Yeast System for Screening Compounds for Effect on Lithium Toxicity

A. Experimental Methods

Characterization of met3Δ and met4Δ yeast Strains of S. cerevisiae in which MET3 (YJR010W) and MET14 (YKL001C) had been deleted in background strain BY4742 were available through the Saccharomyces Genome Deletion Project (Winzeler, 1999). These strains, along with strains representing deletions of virtually every open reading frame in the yeast genome are available to Duke researchers through the Duke Yeast Genome Libraries collection, accessible via the World Wide Web.

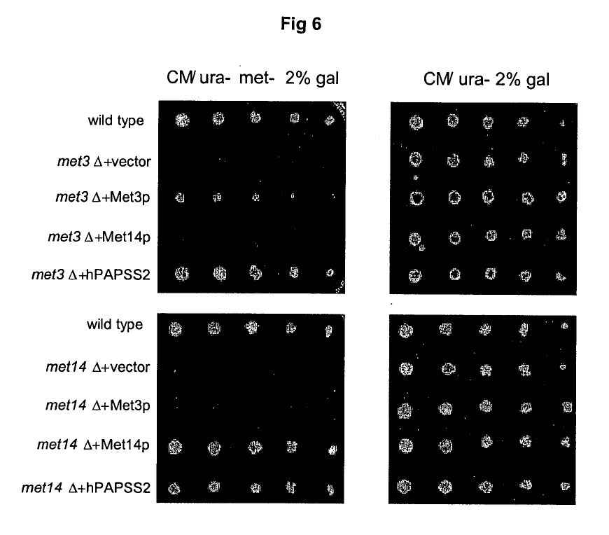

The genotypes were checked via the determination of methionine auxotrophy. Homozygous diploid strains were grown initially in rich media (YPD). To verify that the strains were of the expected genotype, they were washed with sterile water and streaked onto CM or CM/met⁻ agar plates. Deletion strains met3Δ and met14Δ containing an empty vector or an expression vector containing the gene that remained in the genome (met3Δ:pRSMet14p and met14Δ:pRSMet3p) did not grow on media lacking methionine (FIG. 6). This methionine auxotrophy was due solely to the noted disruption because episomal expression of the disrupted gene allowed growth on media lacking methionine (FIG. 6).

In addition to a phenotypic check of the genotypes, genomic DNA was isolated from each of these strains to confirm the genotypes by PCR. Primers used were specific to the G418 cassette or to a region 3′ of the MET3 or MET14. The appearance of PCR products of the expected size (approximately 750 bp) provided additional confirmation of the strains' genotypes.

Cloning and analysis of PAPS synthetases. The two isoforms of human PAPS synthetase were identified in expressed sequence tag databases: EST 601512106F1 (hPAPSS1, accession number BE889854) and EST AL540583 (hPAPSS2, accession number AL540583). The human open reading frames were subcloned by PCR amplification from the EST plasmids. The open reading frames for yeast MET3 and MET14 were subcloned by PCR amplification from wild type genomic DNA from S. cerevisiae strain W303. To clone hPAPSS1, the primers used were 5′ GGA TCC GAG CTC GAA TTC CAC CAT GGA GAT CCC CGG GAG CCT GTG CAA GAA AG (SEQ ID NO: 7) and 5′ GGA TCC GTC GAC GAG CTC GCG GCC GCG GTG GAG TGA CTG GGT TAA CAG CCT AAG C (SEQ ID NO: 8). To amplify hPAPSS2, the primers were 5′ AGA TCT TTC AAT TGA AGC TTG TCG ACC AGC ATG TCG GGG ATC AAG AAG C (SEQ ID NO: 9) and 5′ AGA TCT AAG CTT CCG CGG TCG ACC TGG AGC CAA AGG CTT AGT TCT Tcu (SEQ ID NO: 10). To amplify MET3, the primers were 5′ GGA TCC ATG CCT GCT CCT CAC GGT GGT ATT C (SEQ ID NO: 11) and 5′ CCG CGG TCG ACG CGG CCG CGG TCG ATC ATG AAT TTT GCC CTA C (SEQ ID NO: 12). To amplify MET14, the primers were 5′ GGA TCC AAG CAC ACT GTA CAC CAA TGG CTA C (SEQ ID NO: 13) and 5′ GCG GCC GCC GCG GTC GAC CGG ATC AGA ATT TCA CGG TAA TCC (SEQ ID NO: 14). In each case, the underlined sequence corresponds to sequence surrounding the open reading frame of interest.

The amplified products were gel purified and cloned into pCR2.1 using the TA cloning kit (Invitrogen; San Diego, Calif., United States of America). Each of the pCR2.1 clones was sequenced on both strands by fluorescent terminator sequencing (Howard Hughes Medical Institute, Duke University Medical Center, Biopolymer Facility, Durham, N.C.). The predicted open reading frames obtained from the sequencing data were 100% identical to published sequences (accession numbers: hPAPSS1 XM_{—}052687, hPAPSS2 AF074331, MET3×06413, and MET14×57990.) The hPAPSS1, MET3, and MET14 open reading frames were digested from the pCR2.1 vectors with BamHI, while the hPAPSS2 open reading frame was released by digestion with BglII. Following gel purification, the inserts were ligated into BamHI-linearized pRS426GAL yeast expression vector. The resulting plasmids and control vector were transformed into met3Δ, met14Δ, or wild-type BY4742 yeast using a lithium acetate protocol as described (Stolz, 1998a). Yeast carrying the plasmids were selected for by growth on CM/ura⁻ containing 2% dextrose.

Investigation of methionine auxotrophy was performed by spot dilution assays on solid media. Cells were grown to mid-log phase in CM/ura⁻ containing 2% raffinose when 2% galactose was added to induce gene transcription. Following 6 hr induction, the cells were harvested by centrifugation and washed several times with sterile water. The cells were counted with a hemacytometer and diluted in water to 1×10⁴ cells per microliter. This suspension was serially 5-fold diluted four times, and 1 μl of each dilution was plated on CM/ura⁻ or CM/ura⁻/met⁻ containing 2% galactose. The plates were incubated at 30° C. for two days prior to imaging.

Measurement of intracellular PAP concentrations. A novel isotope binding competition assay was developed to measure intracellular concentrations of PAP. The concentration of PAP in lysates was determined by the binding of radiolabeled PAP to the enzyme phenol (aryl) sulfotransferase (PST), which uses PAP as a cofactor (Yang, 1996b). IMAGE consortium clone 1924316 (accession number A1316417) was found to share significant identity with mouse PST 1A (Sult1a1) (accession number XM_{—}133839). The open reading frame was amplified by PCR using the primers 5′ ATC GAT CAT ATG GAG CCC TTG CGT AAA CCA C (SEQ ID NO: 15) and 5′ TCA TAT TTG ACA GCG GAA CGT G (SEQ ID NO: 16) where the underlined sequences correspond to sequence in the PST open reading frame. The PCR product was gel purified and ligated into the pCR2.1 vector. The insert was sequenced on both strands to ensure fidelity of amplification. The PST open reading frame was subcloned into the BamHI and NotI sites of pGEX4T-1 to create a bacterial expression vector in which PST was expressed in frame downstream of GST (GST-PST). GST-PST was expressed and purified essentially as described by the manufacturer of the glutathione-sepharose resin (Amersham Biosciences, Piscataway, N.J., United States of America). Following application of the GST-PST-containing crude bacterial extract, the resin was washed extensively with K-lysis buffer. MgCl₂ was added to 5 mM, and recombinant human BPntase (typically 1 μg) was added to digest residual bacterial PAP and PAPS that remained bound to PST. The resin again was washed extensively with K-lysis buffer, and GST-PST was eluted with 10 mM reduced glutathione. Pure GST-PST was dialyzed approximately 20,000-fold against 50 mM HEPES, 50 mM KCl, pH 7.5 (K-lysis buffer).

For the assay of intracellular PAP concentrations, appropriate yeast strains were grown in CM/ura⁻ to mid-log phase (OD₆₀₀=1.0). The cells were centrifuged and washed extensively in sterile water. The cells were resuspended in CM/ura⁻/met⁻ containing methionine (20 μg/ml), lithium, or chlorate as appropriate. The cultures were incubated with shaking at 30° C. At various time points, 2 ml of culture was removed and centrifuged. The cells were washed with 1 ml of water. 250 μl was centrifuged and resuspended in 200 μl K-lysis buffer. 200 μl of glass beads were added, and the cells were disrupted by vigorous bead beating. The amount of soluble protein in this extract was determined using a Bradford dye-binding assay (BioRad, Hercules, Calif., United States of America) with bovine serum albumin as a standard. The remaining 750 μl of washed cells was spun down and resuspended in 250 μl of 2 N HClO₄. This was incubated at 4° C. for 20 min, when 250 μl of 1.8 N KOH, 0.4 N KHCO₃ were added to neutralize and precipitate the HClO₄. The extract was centrifuged at 4° C. to remove the debris, and the supernatant was lyophilized using a rotary evaporator. The dried extracts were frozen at −80° C. for no more than one week prior to use. Extracts were reconstituted in 100 μL of 200 mM HEPES, pH 7.5 and again centrifuged to remove debris. 5′ [³²P] 3′, 5′ PAP was created as described (Spiegelberg, 1999). The GST-PST assay was performed by mixing 10 μL of standard PAP, treated as above, or 10 μL of sample diluted approximately 1:1000 in K-lysis buffer, with 10 μL of radiolabeled PAP (approximately 10,000 CPM). Eighty microliters of a reaction mix containing 100 mM HEPES, pH 7.0, 5 mM DTT, 0.2 mg/ml BSA, and 1 μg of GST-PST were added to start the binding reaction. The reactions were allowed to equilibrate for at least one hour at room temperature. Following incubation, 20 μl of a 50:50 slurry of glutathione sepharose in K-lysis buffer was added. The tubes were incubated with gentle mixing for 10 min at room temperature. The protein with bound PAP was pulled down by centrifugation for 2 min at room temperature. 80 μl of the supernatant, containing free PAP, was removed to a scintillation vial and 200 μl of K-lysis buffer was added to wash the resin. The tubes were centrifuged again and the 200 μl wash was transferred to the original scintillation vials. Radioactivity was determined by adding 5 ml of SCINTISAFE ECONO2™ scintillation fluid (Fisher Scientific, Hampton, N.H., United States of America) and counted in a liquid scintillation counter. The counts were converted to percent isotope bound, and a standard curve was prepared by plotting percent bound vs. the logarithm of the PAP concentration. This standard curve was used to back-calculate the PAP concentration in the unknown samples.

B. Yeast-Based Assays for Compounds Effecting Lithium Toxicity

Since methionine effects transcriptional down regulation of the MET3 and MET14 gene products in wild type yeast, the relative lithium sensitivities of the met3Δ and met14Δ strains were assayed in growth media containing reduced methionine (1.25 μg/ml). The use of limited methionine allowed for the comparison of the relative sensitivity to lithium of yeast expressing (wild type) or lacking (met3Δ and met14Δ) PAPS synthetase activities.

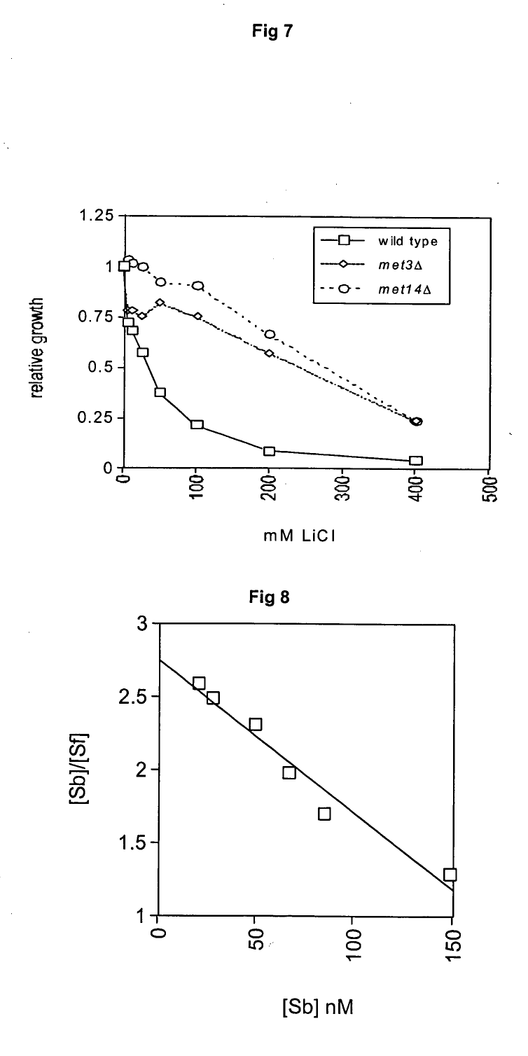

Since a low concentration of methionine was utilized, the methionine auxotrophic strains showed inhibited growth at all lithium concentrations relative to that of wild type. However, when the growth (OD₆₀₀) at 24 hr was plotted relative to maximum growth (growth in media lacking lithium) of a particular strain, it is clear that both met3Δ and met14Δ exhibited lithium resistance as compared to wild type (FIG. 7). The IC₅₀ of lithium for the wild type strain was approximately 40 mM, but the IC₅₀ of lithium for the met3Δ and met14Δ strains was significantly higher, at approximately 200 mM (FIG. 7).

To prove that PAP synthesis was abolished in met3Δ and met14Δ strains, a novel technique to measure PAP concentrations from yeast in suspension was developed. Following acid lysis to halt enzymatic PAP degradation, the PAP concentrations in neutralized lysates were determined using competition with radiolabeled PAP for binding to mouse phenol-preferring sulfotransferase expressed as a fusion to GST (GST-PST). The amount of radioactivity bound to GST-PST was determined by immobilizing the protein to glutathione-sepharose resin. As a check of the system, this procedure was used to determine the dissociation constant of PAP with GST-PST. The observed K_{d} was approximately 35 nM (FIG. 8), consistent with the apparent K_{d} found for the non-fusion recombinant rat PST (Yang, 1996b). Note that PAPS is acid-labile, and the cells to be analyzed are lysed in a strong acid solution; therefore, this technique measures the sum of intracellular PAP and PAPS. However, due to its instability, the intracellular concentration of PAPS is thought to be relatively minor compared to the intracellular concentration of PAP (Jakubowski, 1993).

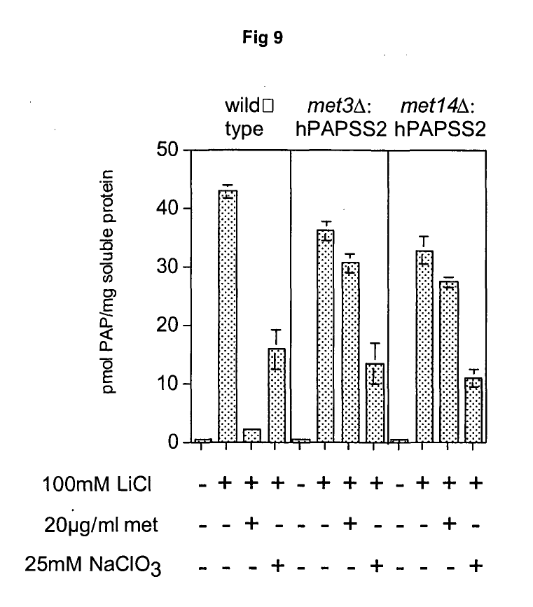

The GST-PST binding assay was used to determine relative intracellular PAP concentrations in yeast strains grown under various conditions. The PAP concentration determined in the binding assay was normalized to the total soluble protein in parallel extracts, and background was eliminated by reporting only the signal that was sensitive to in vitro degradation by purified recombinant Hal2p. The assay was characterized further by analyzing the intracellular PAP concentration of wild type yeast grown in the presence of lithium. The PAP concentration in yeast cells grown in the absence of lithium was found to be below the detectable limits of the assay (approximately 0.5 nmol PAP/mg soluble protein). However, when the cells are treated for 8 hr with 100 mM LiCl in the absence of methionine, the PAP concentration increases to 42.9 nmol/mg, an increase of at least 85.8-fold (FIG. 9), which is consistent with the increase found in previous analyses (Murguia, 1996). Moreover, when methionine (20 μg/ml) was included in the media, the concentration of PAP increased to only 2.3 nmol/mg in the presence of lithium (FIG. 9). This suppression of lithium-induced PAP accumulation by methionine supplementation is consistent with data that suggest that methionine suppresses PAPS synthetase activity (Martin, 1989), decreases PAPS production (Jakubowski, 1993), and moderates the effects of lithium (Dichtl, 1998; Murguia, 1996). The concentrations of PAP in met3Δ and met14Δ cells transformed with an empty vector were found to be below the detectable limits of the assay (less than 0.5 nmol PAP/mg soluble protein), and were found to be independent of the concentration of methionine and lithium in the culture media (data not shown). This indicated that the MET3 and MET14 gene products work together to form the major route of PAP production.

Human PAPS synthetases complement the methionine auxotrophy of met3Δ and met14Δ. In mammals, the ATP sulfurylase and APS kinase activities of Met3p and Met14p are expressed on a single bifunctional enzyme called PAPS synthetase (Li, 1995; Venkatachalam, 1998). Humans express at least two isoforms of PAPS synthetase, hPAPSS1 and hPAPSS2 (Franzon, 1999), which show greater than 80% amino acid sequence similarity. The amino-terminal domain of hPAPSS1 has APS kinase activity and shares 65% similarity with S. cerevisiae Met14p (Venkatachalam, 1998), while the carboxyl terminus of hPAPSS1 has ATP sulfurylase activity and shares 28% similarity with Met3p (Venkatachalam, 1998) and strict conservation of active site residues (Ullrich, 2001a; Ullrich, 2001b). In order to test the ability of the human enzymes to functionally replace the yeast activities, hPAPSS1 and hPAPSS2 were subcloned into a galactose-inducible yeast expression vector and transformed into yeast strains in which either MET3 or MET14 had been disrupted.

IMAGE Consortium Expressed Sequence Tag clones were discovered based on sequences corresponding to the 5′ end of the published sequences of the hPAPSS mRNAs, and appropriate restriction sites were added via PCR as described herein. The constructs were ultimately subcloned into the pRS426GAL yeast expression vector. Several clones were isolated and sequenced on both strands to insure fidelity of amplification. The hypothetical amino acid sequences obtained were 100% identical to the published sequences.

To analyze the ability of hPAPSS1 and 2 to complement the yeast activities, the constructs were transformed into homozygous diploid met3Δ: met3Δ and met14Δ:met14Δ yeast. FIG. 6 displays the growth patterns of the various yeast strains. Episomal expression of the deleted gene or heterologous expression of hPAPSS2 rescued growth on met⁻ media in both met3Δ: met3Δ and met14Δ: met14Δ, confirming that the human bifunctional enzyme is sufficient to replace the two PAPS synthesis activities of yeast. The plasmid containing hPAPSS1 was not capable of restoring methionine prototrophy, possibly indicating a problem with the expression of this isoform in yeast.

In addition to the complementation of methionine auxotrophy, the ability of hPAPSS2 expression to restore the accumulation of PAP in met3Δ and met14Δ cells was analyzed. The cells were grown to mid-log phase in minimal media containing methionine when they were transferred to media containing various concentrations of methionine and lithium. Following growth for eight hours, the cells were lysed and analyzed for intracellular PAP concentration. As shown in FIG. 9, expression of hPAPSS2 restored PAP accumulation in the presence of lithium approximately 36.2 nmol PAP/mg protein in met3Δ and 32.8 nmol/mg in met14Δ, near wild type levels. Methionine had little effect on PAP accumulation due to the fact that the PAPS synthetase was expressed on a galactose-inducible vector. The methionine insensitivity of the activity of the gene products expressed from an inducible promoter supports the idea that methionine regulates the activities of Met3p and Met14p through a transcriptional mechanism.

A pharmacological reduction of PAP-mediated lithium toxicity. The lithium resistance conferred by genetic deletion of PAPS synthetase activity supports a model by which activity of the sulfur assimilation pathway is essential for lithium toxicity in a eukaryotic system. The discovery of a mammalian BPntase suggested that PAP could play a role in the effects of lithium in higher organisms as well and that these effects could be reduced by inhibition of PAPS synthetase activity. To develop a tool to suppress PAPS synthetase that could be translated to a mammalian system, a pharmacological reagent to inhibit the activity of the enzymes was investigated. The inorganic ion chlorate was a candidate for this role since it is known to be a specific inhibitor of ATP sulfurylase activity in mammalian cells (Baeuerle, 1986) and the in vitro activity of the yeast ATP sulfurylase Met3p (Foster, 1994; Ullrich, 2001b). As such, chlorate has been used as a reagent to specifically decrease levels of PAPS in mammalian cells to look for effects of blocking sulfurylation of biomolecules on cellular processes (for example, Chang, 1998; Girard, 1998; Schriever, 1997).

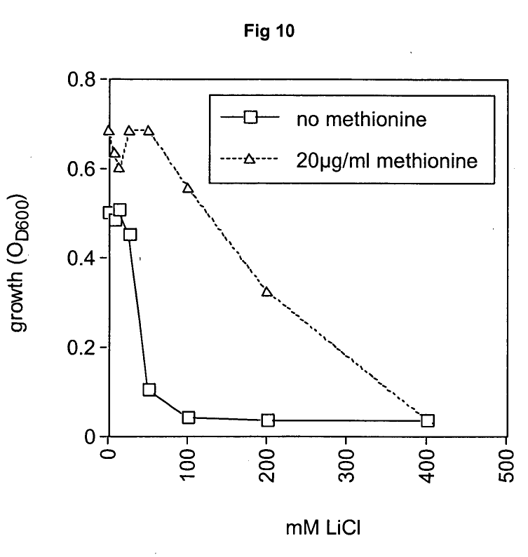

The yeast system was used to determine if chlorate could be used to effect a relevant decrease in the accumulation of PAP in the presence of lithium. Hence, intracellular concentrations of PAP again were determined in cells grown under various conditions. In the presence of 100 mM lithium, the concentration of PAP in wild type cells or met3Δ or met14Δ cells expressing hPAPSS2 increased from undetectable in the absence of lithium (at most 0.5 nmol/mg protein) to 42.9 nmol/mg, 36.2 nmol/mg, and 32.7 nmol/mg, respectively, increases of at least 85.8-, 72.4-, and 65.4-fold (FIG. 9). On the other hand, when these strains were incubated with 25 mM NaClO₃, the fold PAP accumulation was reduced to 30.8, 27.0, and 22.1 (FIG. 9), indicating that ClO₃⁻ indeed inhibited the sulfur assimilation pathway in these cells. The decrease in PAP accumulation was physiologically relevant since chlorate produced a protective effect in the presence of lithium. Wild-type cells grown in the absence of lithium are growth-inhibited with an IC₅₀ of approximately 40 mM (FIGS. 7, 10 and 11). When 25 mM NaClO₃ is included in the culture medium, the growth inhibition is abated, with the IC₅₀ increasing to approximately 80 mM LiCl (FIG. 11)

### Example 2

### General Organ Localization and Kidney Sublocalization of BPntase

The expression patterns of the BPntase enzyme in various mammalian tissues was examined. Lithium's diverse physiological effects on the body include a therapeutic effect arising from an interaction with the nervous system and toxic effects on the brain, the digestive system, and the kidney. Combined with the limited knowledge of the function of nucleotidase activity in the yeast system, the expression of BPntase in mammalian tissues was analyzed in order to develop a model of the role of BPntase in both normal physiology and in the physiology of patients undergoing lithium therapy.

A. Experimental Methods

Northern Blot Analysis.

Approximately 30 ng of the 430 bp StyI fragment of human BPntase cDNA was radiolabeled with [γ³²P] ATP to a specific activity of approximately 1×10⁹ cpm/μg as described with the Random Hexamer Primer Labeling Kit (Boehringer Mannheim; Indianapolis, Ind., USA). A human multiple tissue Northern blot, prepared with 2 μg of poly(A) selected mRNA isolated from various tissues, was purchased from Clontech (Palo Alto, Calif., United States of America). The membrane was prehybridized and hybridized at 42° C. in 10 ml of 50% formamide/5×SSPE (0.9 M NaCl/50 mM phosphate, pH 7.4/5 mM EDTA)/10× Denhardt's solution/0.1 mg of sheared salmon sperm DNA per ml/2% SDS. The membrane was washed twice with 2× standard saline citrate (SSC)/0.05% SDS at room temperature and twice with 0.1×SSC/0.1% SDS at 50° C. and then exposed to film. The membrane was stripped and reprobed with radiolabeled human actin DNA supplied with the blot. The signal was visualized by exposure of BIOMAX™ MR film (Kodak, Rochester, N.Y., United States of America) and was quantified using a phosphorimager device. The signal strength resulting from the BPntase probe in each lane was normalized the to the actin signal to control for total RNA loaded.

Tissue Preparation.

Kidneys were dissected from CO₂-euthanized adult mice and immediately fixed in 4% paraformaldehyde in PBS for 24 hr at 4° C. Following fixation, paraformaldehyde was removed and replaced with 70% ethanol. The tissues were embedded in paraffin, and 10 μm slices were prepared by the Duke University Medical Center Department of Pathology, Durham, N.C., United States of America. Slices were stored at room temperature for less than three months prior to use.

Antibody Purification.

Rabbit-derived antibodies against full-length mouse BPntase were described previously (Spiegelberg, 1999). Specific antibodies were purified according to an established protocol (Harlow, 1988). Briefly, recombinant BPntase was immobilized to CNBr-activated sepharose (Sigma, St. Louis, Mo., United States of America). Typically, 11 mg of BPntase was immobilized to 1 mL of resin. Fifteen milliliters of crude serum was applied to 1 mL of BPntase-conjugated resin. The resin was washed extensively in PBS. Antibodies were initially eluted with 10 mM glycine, pH 3.5. The column was reequilibrated to pH 8.5 with 100 mM Tris-Cl. A second elution of antibodies was achieved by washing the column with 10 mM triethylamine, pH 11.0. Low and high pH elutions were combined, and the pH was titrated to neutrality with 100 mM Tris-Cl, pH 8.0. Sodium azide (0.02% w/v) was added, and the antibodies were aliquoted and frozen at −80° C. Monoclonal antibodies to C-terminal peptides of rat aquaporin-2 and rat Na⁺, K⁺, 2 Cl⁻-cotransporter were obtained from Dr. James Wade, University of Maryland Baltimore County, United States of America.

Immunohistochemistry.

Tissue slices were probed using the VECTASTAIN™ Kit (Vector Laboratories, Burlingame, Calif., United States of America) with slight modifications of the manufacturer's protocol. Paraffin was removed from the slices with two 7.5 min incubations in HEMO-DE™ Clearing Agent (Fisher Scientific, Hampton, N.H., United States of America). The slices were rehydrated with two 5 min incubations in 100% ethanol, 5 min in 95% ethanol, 5 min in 70% ethanol, and two 5 min incubations in water. Endogenous peroxidase activity was blocked by incubating the sections in 0.5% H₂O₂ (v/v in H₂O) followed by three 5 min washes in PBS. Non-specific protein binding sites were blocked by a 1 hr incubation with 1% (w/v) bovine serum albumin (BSA) in PBS at 37° C. in a humidified chamber. The sections were incubated with primary antibody at dilutions of 1:10 to 1:100 as noted in 1% BSA/PBS for 2 hr at 37° C. in a humidified chamber. Unbound primary antibody was removed with three 5 min room temperature PBS washes. The Vectastain secondary antibody was applied at a dilution of 1:200 in 1% BSA/PBS for 30 min at 37° C. in a humidified chamber. Following three 5 min PBS washes, VECTASTAIN™ Elite ABC reagent was applied. This reagent was created by diluting reagents A and B 1:50 in PBS and incubating the mixture for 30 min at room temperature prior to use. The sections were incubated with the Elite ABC reagent for 30 min at 37° C. in a humidified chamber. Elite ABC-applied sections were washed as above, and the stain was developed with 0.5 mg/mL 3,3′-diaminobenzidine tetrahydrochloride (DAB) and 0.01% H₂O₂ in 250 mM Tris-Cl pH 7.4. Staining was allowed to proceed at room temperature until the desired contrast was achieved, typically 5 min. The staining reaction was stopped by extensive rinsing in H₂O. Following staining, slides were preserved by dehydration. The slides were incubated for 5 min in 70% ethanol, 5 min in 95% ethanol, and 5 min in 100% ethanol followed by two 7.5 min incubations in HEMO-DE™ agent. Permount mounting solution (Fisher Scientific, Hampton, N.H., United States of America) and glass coverslips were applied. Results were visualized on an Olympus Vanox AHBS3 Microscope (Melville, N.Y., United States of America), and pictures were taken using a RT Color Spot camera (Diagnostic Instruments, Inc., Sterling Heights, Mich., United States of America).

Tissue Extracts and Western Blotting.

Cortex, outer medulla, and inner medulla were dissected from freshly-harvested rat kidneys and were immediately placed into lysis buffer (50 mM HEPES, 50 mM KCl, 1 mM PMSF, pH 7.5) and lysed with a homogenizer. Extracts were centrifuged at 4° C. for 10 min, and the supernatants were immediately frozen. Mouse inner medullary collecting duct cells (mIMCD-3) (Rauchman, 1993) were grown in DMEM:F12 50:50 (Invitrogen, Carlsbad, Calif., United States of America) containing 10% heat-inactivated fetal bovine serum (Sigma, St. Louis, Mo., United States of America) and penicillin and streptomycin. The cells were maintained in a humidified 37° C. incubator in a 5% CO₂ atmosphere. Cells were washed with HEPES-buffered saline (25 mM HEPES, 125 mM NaCl, pH 7.4), scraped from the plate with a rubber policeman, and sonicated in lysis buffer. Homogenates were spun at 20,000×g in a 4° C. microcentrifuge for 30 min. PAP hydrolysis assays and Western blots of extracts were performed as previously described (Spiegelberg, 1999).

B. Role of BPntase in the Kidney

The localization study described above led to a model in which BPntase is involved in the homeostasis of body salt and water levels through nucleotide metabolism in the kidney. In that the kidney is an important toxic target of lithium and that bisphosphorylated nucleotides are known to interact with signaling systems, a testable model was put forth in which inhibition of BPntase results in the commonly observed side effect lithium-induced nephrogenic diabetes insipidus (NDI). The results presented herein reveal a role for BPntase in the kidney.

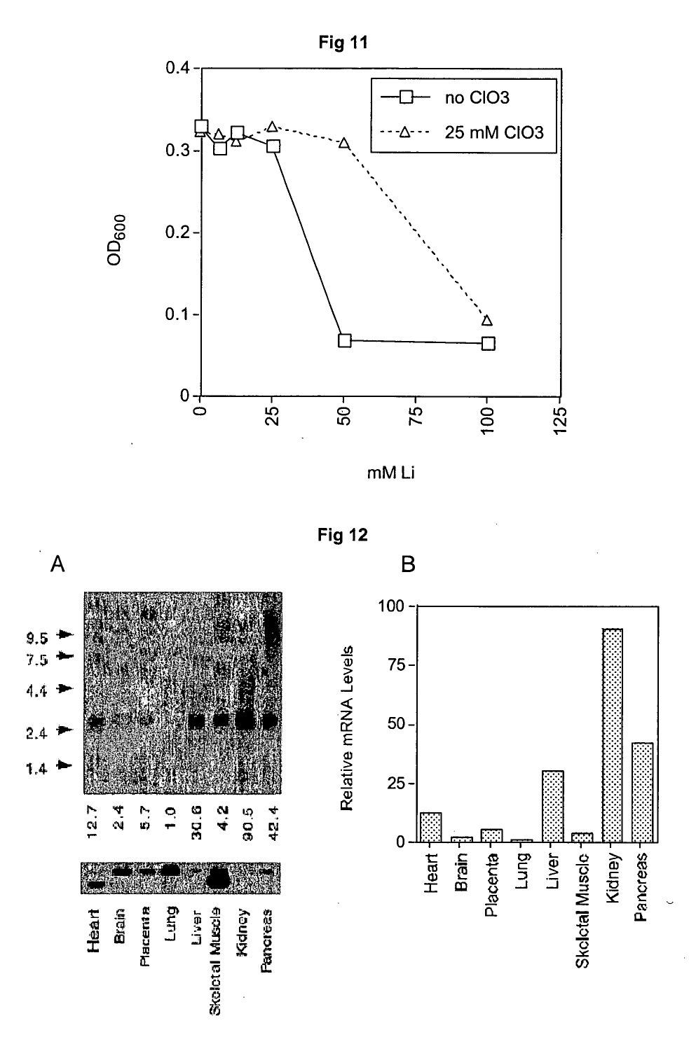

BPntase is enriched in the mammalian kidney. In order to determine the expression pattern and size of the BPntase transcript in human tissues, a multi-tissue Northern blot analysis was performed. A radiolabeled 0.4-kb region of the human cDNA was used to probe 2 μg of human messenger RNA from a variety of tissue sources (FIG. 12A). A single 2.5 kb message was visible at various levels in all tissues examined, indicating that (1) the human EST clone 645079 was approximately full-length and that (2) the message is not specific to any single organ or tissue. As a control for amount of mRNA loaded, the same blot was reprobed with a β-actin probe, as shown in the lower panel. Relative expression was determined by comparing the ratio of human BPntase and actin radioactivity (as quantified by phosphorimage analysis). The lowest level of expression was observed in lung and was assigned a value of 1.0 arbitrary units to which the other tissues were normalized. The highest level of expression was observed in kidney, which has 90.5-fold increased level relative to lung. Of the tissues analyzed, BPntase signal was most highly enriched in the kidney (90.5 arbitrary units), followed by pancreas (42.4), liver (30.6), heart (12.7), placenta (5.7), skeletal muscle (4.2), brain (2.4), and lung (1.0) (FIG. 12 B).

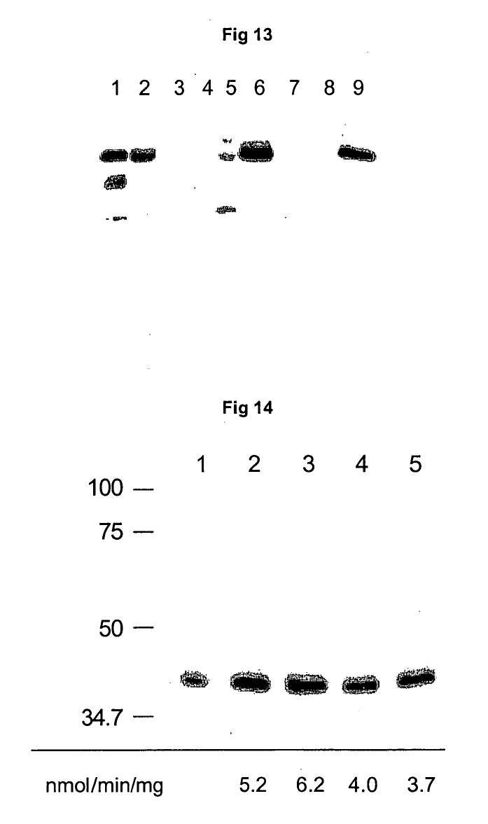

Additionally, analyses of crude mouse tissues using PAP-agarose affinity chromatography and anti-mouse BPntase antibodies were performed to corroborate the mRNA data with protein levels. FIG. 13 displays a Western blot of crude extracts and PAP-agarose-purified elutions of kidney, lung, heart, and liver. Antigen was purified and recognized readily in the kidney and liver extracts but not in extracts of lung or heart. The relative amounts of protein, then, correlate qualitatively with the amounts of mRNA signal observed in the Northern blot analysis.

BPntase is enriched in the proximal tubules and the thick ascending limb of the loop of Henle in the kidney. Quantitative analysis of the tissue distribution results suggested that the enzyme was enriched in the kidney, implying that the protein could have tissue-specific roles. The kidney is a major toxic target of lithium. Considering the known role of yeast bisphosphate 3′-nucleotidase as a lithium target in yeast, BPntase is likely a nephrotoxic target of lithium.

The whole-tissue distribution analyses allowed the localization of relatively high levels of BPntase expression to the kidney. The kidney contains numerous cell types, each of which contributes in a defined way to the urinary concentrating role of the organ. Knowledge of the levels at which each cell type expresses BPntase can play a role in the development of a model of the roles of the enzyme in normal physiology and in lithium treatment.

Western blotting, nucleotidase activity assays, and immunohistochemistry were used to localize expression of BPntase within the kidney. To perform Western blot and activity analyses, cortex, outer medulla, and inner medulla were dissected from rat kidneys. Extracts from these samples were subjected to PAP hydrolysis assays and separated using SDS-PAGE and analyzed via Western blot. As shown in FIG. 14, BPntase was found in all three segments, with a slight enrichment in the outer medulla. The inner medulla consists mainly of collecting tubules, which is the probable location of the target leading to the nephrotoxicity of lithium. Therefore, to confirm expression of BPntase in the inner medullary collecting ducts (IMCD), a cultured cell line derived from the principal cells of the mouse inner medullary collecting duct (mIMCD-3) (Rauchman, 1993) was analyzed. Antigen levels and specific activity of mIMCD-3 extracts were similar to those observed from the dissected inner medulla (FIG. 14), providing further evidence that BPntase is expressed in collecting ducts.

To identify the distribution of BPntase among the various cell types in the kidney, affinity-purified antibodies were used to probe sections of mouse kidneys. Specific staining was detected using a peroxidase-activated precipitating dye that resulted in a visual signal. Fixation and probing conditions were adjusted to obtain significant staining with a relatively low background signal. To acquire the samples for immunohistochemistry, normal adult mice were euthanized with CO₂, and their kidneys were removed. Freshly removed kidneys were fixed and embedded in paraffin. The embedded kidneys were then cut into 10 μm slices and placed on glass slides. Initial analyses were performed by fixing the organs in 10% buffered formalin prior to embedding, but this treatment was found to result in very low specific signal. To improve the signal, fixation conditions were changed to an overnight fixation in 4% paraformaldehyde in PBS. This alteration was apparently effective in that slices treated in this manner showed significant specific staining.

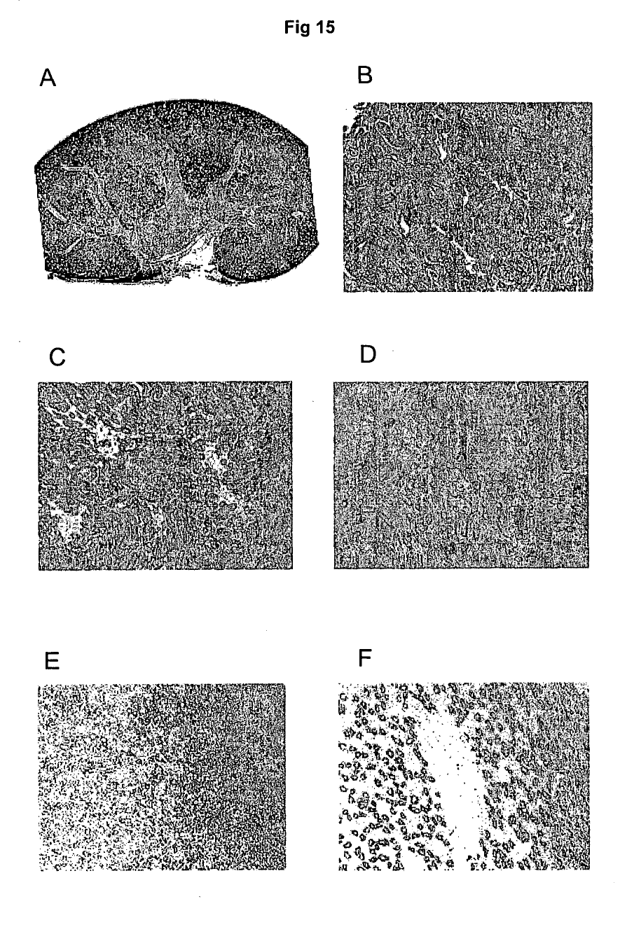

In performing immunohistochemical analyses, additional attention needed to be paid to the treatment of the slices during the probing procedures. A systematic testing of various conditions, including further antigen fixation, antigen unmasking, and cell permeabilization revealed that the results eventually obtained were relatively insensitive to treatment method. This fact was important because it suggested that the results were significant and not merely an artifact of sample treatment. The signal was the strongest relative to background when the slices were fixed and permeabilized with a −20° C. 100% methanol treatment. Antigen unmasking with 6 M guanidine or a variety of detergents was found to be of little consequence to the final results with the anti-BPntase antibody. On the other hand, 4% paraformaldehyde fixation caused an apparent masking of the antigens relating to the integral membrane proteins aquaporin-2 and Na⁺, K⁺, 2Cl⁻-cotransporter 2. Before probing slices for these antigens, the slices were treated with 6M guanidine. As shown in the midline sagittal section displayed in FIG. 15A, the anti-BPntase antibody reacted strongly throughout the cortex and the outer medulla. Closer examination revealed that the antibody stained proximal and distal tubules within the cortex and a single specific tubule type within the outer medulla (FIGS. 15B and 15C). The stained outer medullary tubules were identified as the thick ascending limb of the loop of Henle. This determination was confirmed by staining parallel slices with an antibody against the thick ascending limb-specific rat Na⁺, K⁺, 2Cl⁻-cotransporter (NKCC2) (Yang, 1996a). FIGS. 15C and 15D show that both anti-mBPntase and anti-NKCC2 stained identical tubules, confirming that mBPntase was concentrated in the thick ascending limb. These results correlate with the distribution of antigen and specific activity of the various sections of the dissected rat kidney. In addition, very little staining was observed when the slices were probed with pre-immune serum, suggesting that background staining was low and that the observed signal was specific to the anti-mBPntase antibody. Finally, purified antibodies derived from a second rabbit showed identical staining (data not shown), further supporting the specificity of the signal.

Lithium-induced NDI is thought to arise from an interaction of the drug with the vasopressin (AVP)-activated adenylate cyclase system in the collecting ducts (Discussion, Christensen, 1985; Goldberg, 1988; Jackson, 1980; Yamaki, 1991). Analysis of homogenates of dissected rat kidneys and cultured inner medullary collecting duct cells suggest that BPntase is present in these cells, yet a comparison of serial sections stained with an antibody against rat AQP2, a collecting-duct-specific antigen, (FIGS. 15E and 15F) indicated that the anti-BPntase antibody does not strongly recognize tubules in the inner medulla. BPntase appears to be present in the inner medulla at a low level compared to the expression in the thick ascending limbs and the tubules of the cortex.

BPntase as a toxic target of lithium. The establishment of genetic and biochemical evidence that the sulfur assimilation pathway was the major toxic target of lithium implied a simple tool to mediate lithium's toxicity. Indeed, it was found that chlorate, a known inhibitor of ATP sulfurylase, inhibited production of PAP in vivo. The discovery that BPntase is expressed in the inner medullary collecting ducts in mice thus provides a system to test the efficacy of the antidote effect of a PAPS synthetase inhibitor in a mammalian model of lithium toxicity. The quantitative correlation between a biological effect of lithium, inhibition of cAMP production during stimulation of cultured IMCD cells, and the biochemical effect of lithium on the activity of BPntase, is consistent with BPntase inhibition being a cause of NDI in situ. Chlorate partially restored the accumulation of cAMP, further suggesting the involvement of the sulfur assimilation pathway in the development of NDI.

### Example 3

### Brain and Gastrointestinal Localization of BPntase

Northern and Western blot analyses demonstrated that BPntase is not solely expressed in the kidney, signifying that the physiological role of the enzyme is not merely restricted to this organ and that inhibition of BPntase can contribute to other effects of lithium in vivo. The nervous system is the primary target of lithium, leading both to the diminishing of the symptoms of bipolar disorder and, in the case of lithium poisoning, to coma and potentially death. Lithium therapy also affects the digestive system in that other side effects include nausea and diarrhea.

Immunohistochemistry, Western blotting, and specific activity analyses were performed to characterize the expression of BPntase in tissues other than the kidney. Immunohistochemistry was performed on slices from mouse embryos to obtain a picture of the enzyme's distribution in a whole organism. Intestines from adult mice were analyzed due to the apparent high expression of BPntase in the embryonic gastrointestinal tract. In addition, despite the relatively low expression of BPntase suggested in previous Northern blot analyses, detailed study of the brain was performed to determine if the enzyme was expressed in a substructure of that organ.

Comparative analyses of the expression of an enzyme in various organs can shed insight into physiological roles. This is particularly the case in the study of lithium pharmacology. Therapeutic and toxic effects of lithium administration arise from numerous organs, suggesting that targets of the drug play important roles in these areas. BPntase expression correlates with several known effects of lithium, including a therapeutic effect in the nervous system and toxic effects in the kidney, nervous system, and gastrointestinal tract.

Moreover, the expression patterns of BPntase are consistent with a role for the enzyme in the transport of ions and fluids across cells. In this Example, expression of BPntase was shown to be enriched in epithelial cells of tissues that are involved in fluid flux. Intestinal epithelia are important for the uptake of water and nutrients from a meal as it traverses the gastrointestinal tract. The choroid plexus is the major site in the brain where fluid and materials are exchanged between the plasma and the cerebral spinal fluid contained in the cerebral ventricles.

In light of current knowledge, however, it appears that tissues such as the nephron, the intestinal epithelia, and the choroid plexus could be particularly sensitive to PAP accumulation due to their roles in molecular transport. Alternatively, as suggested by Quintero et al. (Quintero, 1996), a substrate of BPntase activity could act as a regulator of molecular transport, thus explaining the relatively high expression of the enzyme in these tissues.

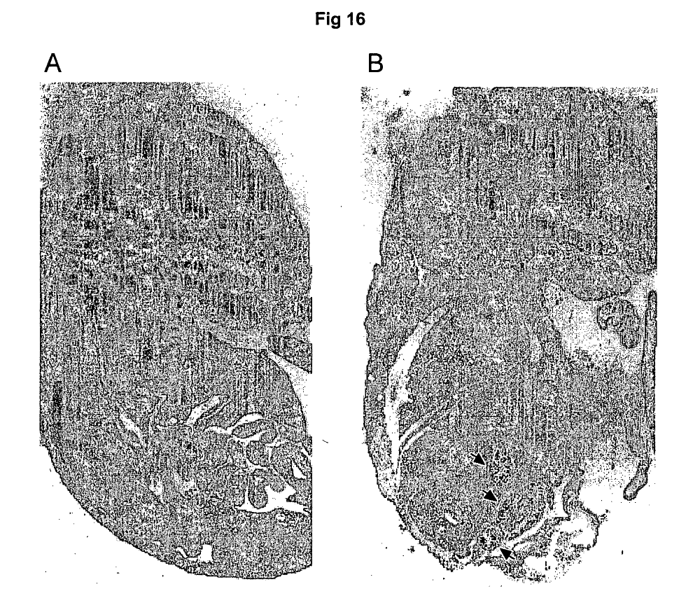

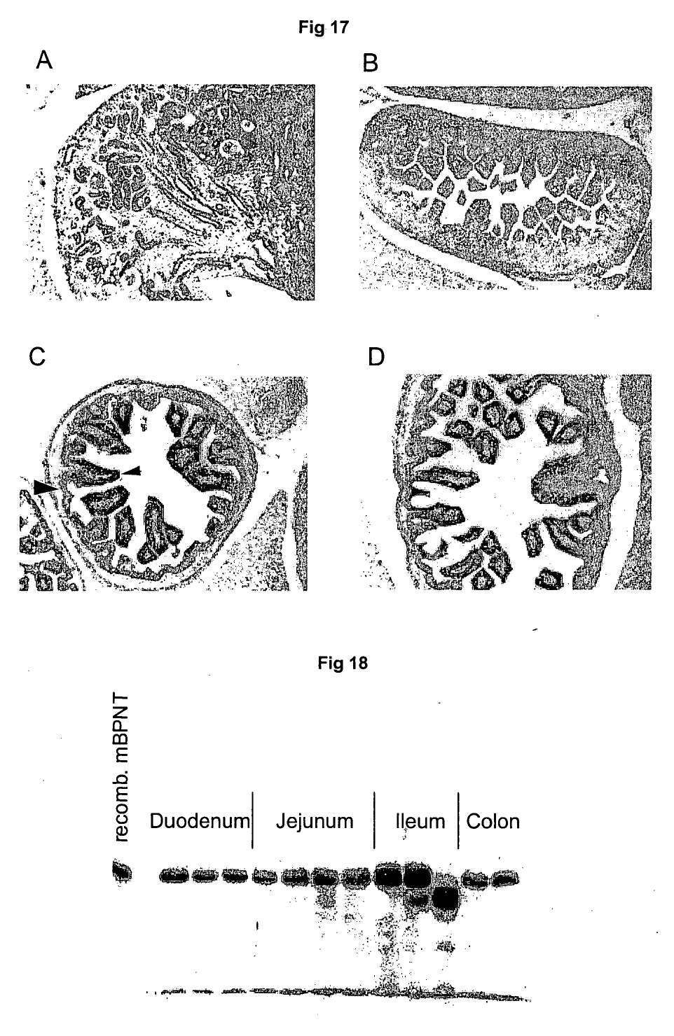

Localization of BPntase in mouse embryos. Kidney localization analyses were extended to determine if BPntase is a potential target of lithium in other organs. As a first step, mouse embryos were analyzed in order to gain a general overview of BPntase distribution in a whole organism. Paraffin-embedded slices of mouse embryos of various ages were obtained from Novagen (an affiliate of Merck KGaA, Darmstadt, Germany) and were probed with affinity-purified antibodies against full-length mouse BPntase. Young embryos (5 to 14 days post coitus) displayed very little staining above that seen with non-specific rabbit IgG or pre-immune serum. However, more mature embryos (days 15 and 16) showed significant staining in several regions (FIGS. 16A and 16B). As expected from distribution studies performed on adult tissues, significant BPntase staining was seen in the immature tubules of the day 16 embryonic kidney (FIG. 17A). Unexpectedly, the most striking signal in day 16 embryos was localized to the gastrointestinal tract (FIG. 16B), a tissue that was not analyzed in earlier Northern or Western blot studies. Two characteristics are noted upon examination of high magnification views of embryonic intestinal staining (FIGS. 17B-17D). First, the intensity of staining increases greatly upon transition from day 15 (FIG. 17B) to day 16 (FIG. 17C and 17D), suggesting that BPntase expression in the intestine increases as the mouse approaches birth. Second, staining is concentrated in cells at the tips of the intestinal villi, and there is a relative dearth of staining in the crypts of Lieberkuhn (FIGS. 17C and 17D), suggesting that BPntase expression increases as cells mature and become differentiated epithelial cells.

Intestines were dissected from adult mice to confirm high-level expression of BPntase. Mice were deprived of food for at least 12 hr to eliminate material from the digestive tract, sacrificed via CO₂, and dissected to remove the gastrointestinal tracts. The various sections of the small intestine and the colon were dissected. Western blot (FIG. 18) and activity assays of resulting lysates revealed that BPntase was expressed in all segments of the gastrointestinal tract but that antigen and activity were relatively enriched in the ileum of the small intestine (Table 2). Distribution of specific activity of 3′-nucleotidase activity along the intestine was duodenum, 3.8 nmol of substrate converted per minute per mg of crude protein, jejunum, 6.8, ileum, 10.4, and large intestine, 2.7 (Table 2). These analyses were complicated by the observation in the Western blot that the BPntase antigen was degraded, especially in the latter part of the ileum (FIG. 18), suggesting that the actual specific activity could be higher than observed. Bisphosphate 3′-nucleotidase specific activities of the noted sections of the intestines were determined and are displayed in Table 2.

TABLE 2
Summary of BPntase distribution. The distribution of BPntase in
various tissues was determined by immunohistochemistry (IHC)
and specific activity of extracts. Specific activities were
determined at 1 μM PAP. All tissues described are from adult
mouse except for (a), which was determined for adult rat kidneys,
(b) which was determined for embryonic (d16 post-coitus) mouse,
and (d) which was derived from neonatal rat. The specific
activities of extracts from the whole gastrointestinal tract and
choroid plexus and the IHC signal of the individual sections of the
adult mouse gastrointestinal tract were not determined (c: N.D.).

| | | | Specific Activity |
|---|---|---|---|
| Organ | Tissue | IHC Signal | Nmol/min/mg |
| Kidney | Cortex | ++ | 5.2^{a} |
| | Outer Medulla | +++ | 6.2^{a} |
| | Inner Medulla | +/− | 4.2^{a} |
| Gastrointestinal | Whole^{b} | ++++ | N.D.^{c} |
| Tract | Duodenum | N.D. | 3.8 |
| | Jejunum | N.D. | 6.8 |
| | Ileum | N.D. | 10.4 |
| | Colon | N.D. | 2.7 |
| Brain | Whole | + | 0.16 |
| | Choroid Plexus | +++ | N.D. |
| | Cortical Neurons^{d} | + | 0.22 |

Localization of BPntase in Mouse Brain.

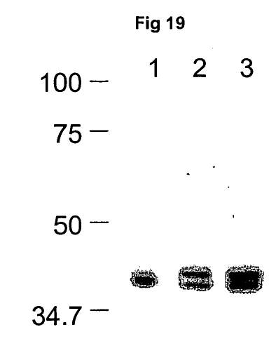

A potential role of BPntase in the therapeutic effects of lithium is of great interest. Levels of BPntase message in a Northern blot of human tissues were found to be significant in the brain, albeit approximately 40-fold lower than the highest-expressing organ, the kidney. In addition, PAP hydrolysis assays of homogenized whole mouse brain showed a specific activity of 0.16 nmol/min/mg (Table 2) and Western blot analyses clearly showed the presence of BPntase antigen (FIG. 19). Thus, while overall levels of BPntase in the brain are below those of the kidney, specific localization within the brain can suggest models by which BPntase inhibition impinges on lithium therapy. Since the anti-BPntase antibodies were shown to be effective in immunohistochemical analyses of kidneys and embryos, these reagents were used to investigate the distribution pattern of BPntase in mouse brain.

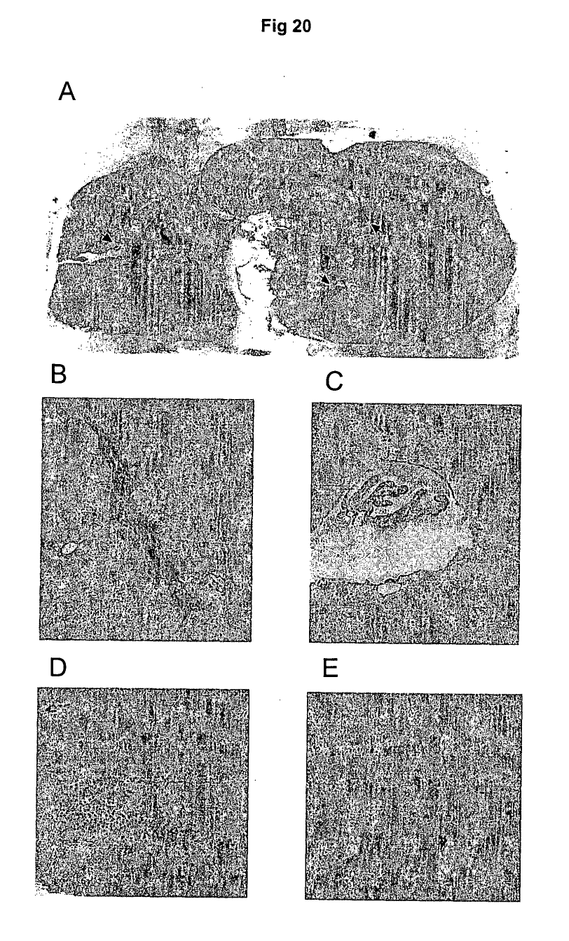

Since BPntase was clearly expressed in the brain, its distribution within the organ was investigated by probing mouse brain sections with affinity-purified anti-mBPntase antibodies. As shown in FIG. 20, staining was less intense than that seen in the kidney, but it was generally distributed throughout the brain. In the low magnification view of a midline sagittal section, two definite areas of intense staining were apparent (FIG. 20A). These areas are shown at higher magnification in FIGS. 20B and 20C. The staining in these regions, which was absent when the slices are stained with non-specific IgG controls, was identified as the choroid plexus of the third and fourth ventricles based on location and morphology. Analysis of sagittal slices further from the midline indicated that BPntase staining was also present in the choroid plexus of the lateral ventricles.

In addition to the cells of the choroid plexus, neuronal cell bodies were also stained throughout the brain (FIG. 20A), especially in the hippocampus (FIG. 20D) and the cortex (FIG. 20E). To confirm this observation, neurons were analyzed separately from other structures within the brain. Cultured rat cortical neurons were a generous gift of Dr. Michael Ehlers in the Department of Neurobiology, Duke University Medical Center, Durham, N.C., United States of America. The cultures were lysed at day 10 following isolation from neonatal rats. Western blot and specific activity assays confirm the presence of BPntase in neurons and suggest a slight enrichment over the levels of activity in the whole mouse brain (0.22 nmol/min/mg in cultured neurons compared with 0.16 nmol/min/mg in whole brain) (FIG. 19, Table 2). Further confirmation of the expression of BPntase in neurons came from experiments performed that showed through in situ mRNA hybridization and immunohistochemistry that the enzyme is expressed in cortical and hippocampal neurons in human and macaque brains. These analyses revealed especially high expression of BPntase in the dentate gyrus of the hippocampus.

### Example 4

### Elucidation of PAP-Interacting Proteins

When pig kidney extracts were pre-cleared by passage over both cation and anion exchange columns, three bands appeared upon SDS-PAGE analysis following PAP-agarose chromatography of the eluate. The bands, migrating at approximately 18-, 39-, and 42-kDa, were submitted for sequencing via Edman degradation. Preliminary results revealed two potential PAP-interacting proteins. The band at 39-kDa was not sequenced, probably due to an N-terminal modification, but it reacted with the anti-mBPntase antibodies and was presumed to be the pig BPntase. The band at 18 kDa was also N-terminally blocked but might be NDP kinase, which is known to bind PAP. Sequencing of the 42 kDa band revealed it to be betaine-homocysteine methyltransferase (BHMT).

BHMT is a central enzyme in the regulation of methionine biosynthesis and the metabolism of homocysteine. Methionine can be synthesized by the BHMT-catalyzed transfer of a methyl group from betaine to homocysteine. Interestingly, BHMT expression is enriched mainly in the mammalian liver and kidney (Sunden, 1997), two organs that express relatively high levels of BPntase (FIG. 12). In addition, a putative nucleotide binding sequence on the protein has been localized but not explained (Garrow, 1996; Sunden, 1997), leaving open the possibility that PAP is a regulator of the enzyme's activity.

### Example 5

### Bpntase Knockout Mouse

Materials and Methods

I. BPntase Targeting Construct

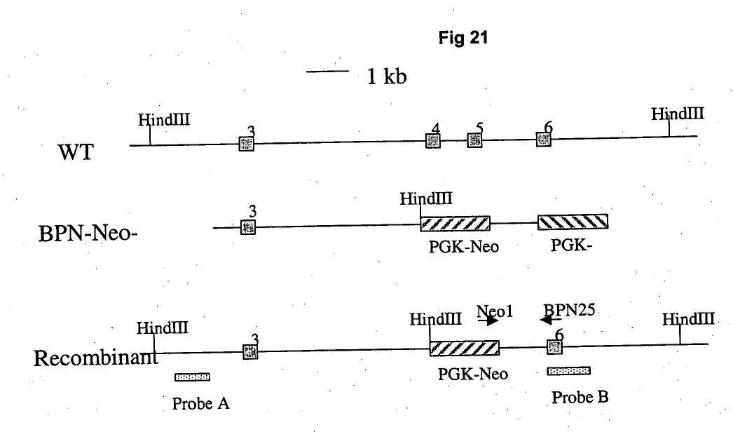

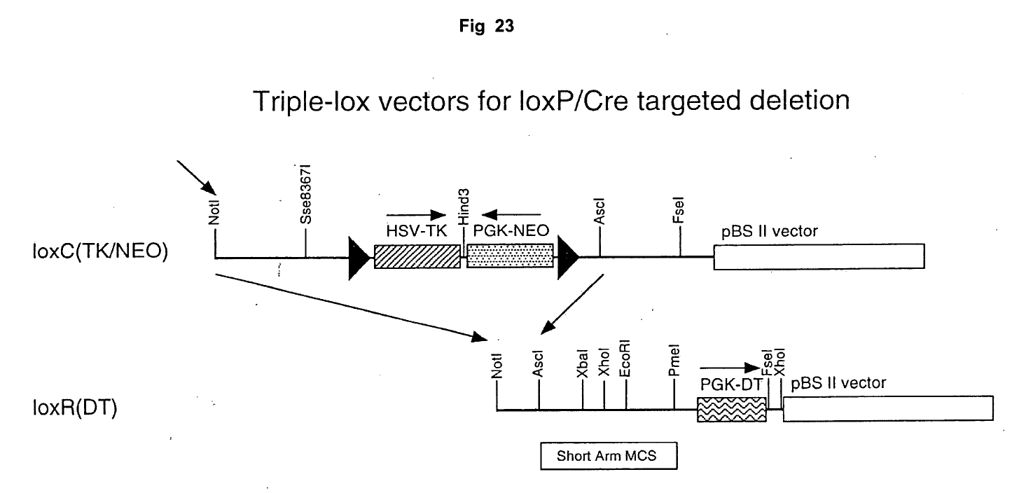

A knockout of BPntase targeting vector was generated using genomic DNA and was cloned by PCR using R1 embryonic stem (ES) cell derived mouse genomic DNA as a template. The approach is designed to provide exon removal as is described in greater detail in Gainetdinov, 1999 and Gainetdinov, 2003. See also FIG. 21. The base vectors were highly modified vectors obtained as part of the pTriple-Lox™ ensemble as previously described (Gainetdinov, 1999 and 2003; See also FIG. 23).

Generation of pBSLoxC-LoxR. The pBSLoxC vector (FIG. 23) was digested with NotI and AscI and the resulting 3758 bp TK/PGK-NEO cassette was cloned into these corresponding sites of pLoxR (FIG. 23) to generate pBSLoxC-LoxR.

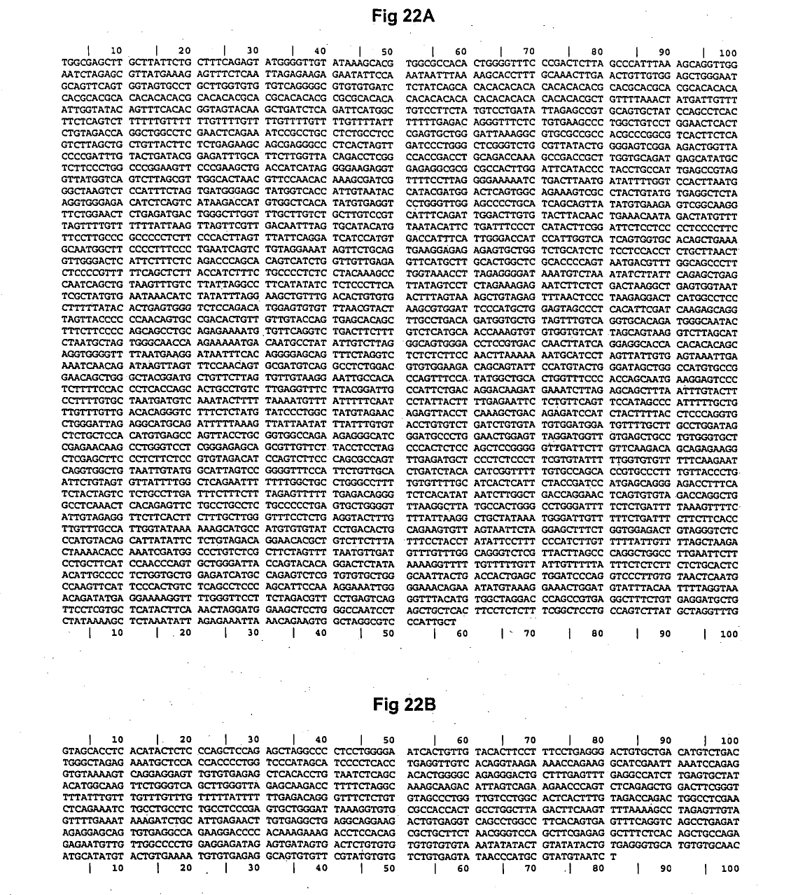

The BPntase ‘short arm’ cloning into pBSLoxC-LoxR. A 982 bp BPntase genomic fragment (SEQ ID NO: 17; see also schematic of FIG. 21 and FIG. 22B) located between exons 5 and 6 was generated by PCR using the below primers and inserted into pCR2.1 TA vector (Invitrogen) to generate pCRBPN9-10.

The 5′ short-arm primer is: LYD009 5′-GGCGCGCCgtagcacctcacatactctcccagctc-3′ (uppercase AscI site, underlined BPNT sequence, SEQ ID NO: 18)

The 3′ short-arm primer is: LYD010 5′-GGCGCGCCagattacatacgcatgggttatactc-3′ (uppercase is AscI site, underlined is BPNT sequence, SEQ ID NO: 19)

The pCRBPN9-10 vector was digested with EcoRI to release the 982 bp short-arm fragment and it was cloned into the EcoRI site of pBSLoxC-LoxR to yield pBSLoxC-LoxR-BPN9-10.

The long arm cloning into pBSLoxC-LoxR-BPN9-10. The long arm (SEQ ID NO: 20; see also schematic of FIG. 21 and FIG. 22A) of BPntase was generated through a PCR cloning strategy with the flanking primers as follows: PrimerLYD001 (5′-primer complementary to 5′ end of long arm—the underlined sequence is compatible with the BPntase genomic and the uppercase denotes the added NotI restriction site): LYD001 5′-GCGGCCGCtggcgagcttgcttattctgctttcag-3′ (SEQ ID NO: 21); and Primer 2 LYD018 (underlined sequence is complementary to the 3′end of the long arm sequence—uppercase denotes added HindIII restriction site):

| | |
|---|---|
| LYD018 | |
| 5′-AAGCTTagcaatgggacgcctagccactt | (SEQ ID NO: 22) |
| ctg-3′. | |

The resulting ‘long-arm’ 4872 bp BPntase fragment (4858 BPNT with 8 bp of NotI and 6 bp HindIII) was cloned into NotI and HindIII sites of pBSLoxC-LoxR-BPN9-10 to yield pBPN-Neo-DT.

II. Generation of Recombinant 129-SVEV Embryonic Stem Cells.

The pBPN-Neo-DT vector was linearized with Not I and delivered to Dr. Randy Thresher of the transgenic facility at the University of North Carolina at Chapel Hill, Chapel Hill, N.C., United States of America. Using well-characterized methods the DNA was transfected into 129-SVEV stem cells and selected with neomycin. Neomycin resistant clones were isolated and tested below for proper recombination as described below.

III. PCR and Southern Blot Analysis of ES Clones.

Two oligonucleotides, BPN25 and Neo1 were used to PCR screen the transfected ES clones. The sequences of BPN25 and Neo1 were 5′-TCCAGCCTTGGGACAAGAGATCAG (SEQ ID NO: 23) and 5′-ACCAAAGAACGGAGCCGGTTGGCG (SEQ ID NO: 24), respectively. Reactions were denatured at 94° C. for 5 minutes, then put through 40 cycles of denaturation at 94° C. for 30 seconds, annealing at 60° C. for 30 seconds and extending at 72° C. for 1 minute. After the 40 cycles, DNA synthesis was completed by incubating the reactions at 72° C. for 5 minutes. Reaction products were analyzed by electrophoresis through 1% agarose gels. The targeted recombinant ES clones gave 1.45 kb fragment.

PCR positive clones were further analyzed by Southern blotting. Ten to fifteen microgram genomic DNA from each clone was digested with HindIII, fractionated by electrophoresis through 0.8% agarose gels, transferred to MAGNA nylon membrane (Osmonics of Minnetonka, Minn. United States of America), and hybridized with probe A or probe B. Probe A is a 1046 bp fragment at the 5′ end of BPntase that yields a 12.7 kb wild type band and a 6.5 kb knock out band in hybridization. Probe B is a 1119 bp fragment at the 3′ end of BPntase that yields a 12.7 kb wild type band and a 6.9 kb knock out band in hybridization. Four independent ES lines were obtained: esBPN2B, esBPN2E, esBPN9G and esBPN10F.

IV. Infection of BPN Recombinant ES Cells into Blastocyst and Chimeric Mouse Generation.

All four ES lines were injected into blastocysts by the transgenic facility at the University of North Carolina at Chapel Hill, Chapel Hill, N.C., United States of America. Litters were obtained and coat-color of offspring was monitored. Five chimeric mice were delivered for breeding (the range was 40 to 90% chimeric as determined by the relative proportion of agouti/black coat color). Chimeric mice were quarantined and bred to obtain germ-line breeding pairs. 2 male +/− and 1 female +/− BPntase mouse lines were developed, and these lines are bred to obtain homozygous null animals.

### REFERENCES

Abrahams et al. (1994) Nature 370, 621-628
Acharya, et al. (1998) Neuron 20, 1219-1229.
Allen et al. (1973) J. Chem. Doc. 13: 119
Allison, J., et al. (1976a) Biochemical and Biophysical Research Communications 71, 664-670.
Allison, J., et al. (1971) Nature New Biology 233, 267-268.
Allison, J. H., et al. (1976b) Biochemical and Biophysical Research Communications 68, 1332-1338.
Altschul, S. F., et al. (1997) Nucleic Acids Research 25, 3389-3402.
Appelt (1993) Perspectives in Drug Discovery and Design 1:23-48
Arrillaga, I., et al. (1998) Plant Science 136, 219-226.
Atack, J., et al. (1995) Trends in Neurosciences 18, 343-349.
Ausubel, F. M., et al. (1995), John Wiley & Sons, Inc., New York.
Baeuerle, P. A., et al. (1986) Biochemical and Biophysical Research Communications 141, 870-877.
Baldessarini, R. J., et al. (2002) Harvard Review of Psychiatry 10, 59-75.
Baraban, J. M. (1994) Proceedings of the National Academy of Sciences: USA 91, 5738-5739.
Barany, 1985 Gene 37:111-23
Bartlett et al. (1989) Special Pub., Royal Chem. Soc. 78: 182-196
Barton et al., 1990 Nucleic Acids Res 18:7349-55
Berridge, M. J., et al. (1989) Cell 59, 411-419.
Birch, N. J., et al. (1973) British Journal of Pharmacology and Chemotherapeutics 47, 586-594.
Blackwell et al. (1995) Mol Med 1:194-205
Boobbyer et al. (1989) J Med Chem 32:1083-1094
Bone, R., et al. (1994a) Biochemistry 33, 9468-9476.
Bone, R., et al. (1994b) Biochemistry 33, 9460-9467.
Bone, R., et al. (1992) Proc. Natl. Acad. Sci. U.S.A. 89, 10031-10035.
Boton, R., et al. (1987) American Journal of Kidney Diseases 10, 329-345.
Bowden, C. (2000) Journal of Clinical Psychiatry 61 (Suppl 9), 35-40.
Bradford, M. M. (1976) Anal. Biochem. 72, 248-254.
Brint & Willett (1987) J Mol Graph 5:49-56
Brummelkamp, T. R., et al. (2002) Science 296, 550-553.
Bugg et al. (1993) Scientific Am Dec:92-98
Cade, J. F. J. (1949) Medical Journal of Australia 37, 349-352.
Cade, J. F. J. (1970) Discoveries in biological psychiatry (Ayd, F. J., Jr., and Blackwell, B., Eds.) pp 218-229, J.B. Lippincott, Philadelphia/Toronto.
Capecchi (1989) Science 244(4910):1288-1292
Chang, C., et al. (1998) Journal of Immunology 160, 4367-4374.
Chen, G., et al. (2000) Journal of Neurochemistry 75, 1729-1734.
Chen, R.-W., et al. (1999) Journal of Biological Chemistry 274, 6039-6042.
Cherest, H., et al. (1971) Journal of Bacteriology 106, 758-772.
Choe, J. Y. et al. (1998) Biochemistry 37, 11441-11450.
Christensen, S., et al. (1985) Journal of Clinical Investigation 75, 1869-1879.
Cohen et al. (1990) J Med Chem 33:883-894
Colicelli et al., 1985 Mol Gen Genet 199:537-9
Cornish-Bowden, A. (1986) FEBS Letters 203, 3-6.
Cwirla et al. (1990) Proc Natl Acad Sci USA, 87:6378-6382
Davson, H., et al. (1987) Churchill Livingstone, New York.
DesJarlais et al. (1988) J. Med. Chem. 31:722-729
Depiereux, E., et al. (1997) Computer applications in the biosciences: CABIOS 13, 249-256.
Devlin (1990) Science, 249:404-406
Diamond, J. H., et al. (1967) Journal of General Physiology 50, 2061-2083.
Dichtl, B., et al. (1998) The EMBO Journal 16, 7184-7195.
Ding et al. (1997) J Biol Chem 272(44):28142-28148
Donowitz, M., et al. (1986) Reviews of Infectious Diseases 8, S188-S201.
Drevets, W. C., et al. (1997) Nature 38, 824-827.
Dunbrack et al. Folding & Design 2:R27-R42.
Dwight, T., et al. (2002) European Journal of Endocrinology 146, 619-627.
El-Mallakh, R. et al. (2001) Harvard Review of Psychiatry 9, 23-32.
El-Mallakh, R. S., et al. (1993) Journal of Neuropsychiatry 5, 361-368.
Elbashir, S. M., et al. (2001) Nature 24, 494-498.
Erickson (1993) Perspectives in Drug Discovery and Design 1:109-128
Fire, A. (1999) Trends in Genetics 15, 358-363.
Fishman, R. A. (1992) Cerebrospinal fluid in diseases of the nervous system, 2nd ed., W. B. Saunders Company, Philadelphia.
Forrest, J. J. (1975) New England Journal of Medicine 292, 423-424.
Foster, B. A., et al. (1994) Journal of Biological Chemistry 269, 19777-19786.
Franzon, V. L., et al. (1999) International Journal of Biochemistry and Cell Biology 31, 613-626.
Fuda, H., et al. (2002) Biochemical Journal 365, 497-504.
Gainetdinov, R. R., et al. (1999) Neuron 24, 1029-1036.
Gainetdinov, R. R., et al. (2003) Neuron 38, 291-303.
Ganzhorn, A. J., et al. (1990) Biochemistry 29, 6065-6071.
Garrow, T. A. (1996) Journal of Biological Chemistry 271, 22831-22838.
Gee, N. S., et al. (1988) Biochemical Journal 253, 777-782.
Geever, R. F., et al. (1989) Neurospora crassa. J. Mol. Biol. 207, 15-34.
Gelenberg, A. J., et al. (1989) New England Journal of Medicine 321, 1489-1493.
Girard, J., et al. (1998) FASEB Journal 12, 603-612.
Glaser, H.-U., et al. (1993) EMBO 12, 3105-3110.
Goldberg, H., et al. (1988) American Journal of Physiology 255, F995-F1002.
Goldberg, J. F. (2000) Journal of Clinical Psychiatry 61 (suppl 13), 12-18.
Goodford (1985) J Med Chem 28:849-857
Graham, D. Y. (1980) New England Journal of Medicine 303, 1063-1064.
Graham, D. Y., et al. (1975) Annals of Internal Medicine 83, 782-785.
Guex, N., et al. (1997) Electrophoresis 18, 2714-2723.
Gustin et al., 1993 Biotechniques 14:22
Hallcher, L., et al. (1980) Journal of Biological Chemistry 255, 10896-10901.
Harborth, J., et al. (2001) Journal of Cell Science 114, 4557-4565.
Hardman, J. G., et al. (2001) The pharmacological basis of therapeutics, 9th ed., McGraw-Hill Medical Pub. Division, New York.
Harlow, E., et al. (1988) Antibodies: A laboratory manual, Cold Spring Harbor Laboratory, Cold Spring Harbor, N.Y.
Harwood, A. J. (2001) Cell 105, 821-824.
Hedgepeth, C., et al. (1997) Developmental Biology 185, 82-91.
Henry, C. (2002) Journal of Psychiatry and Neuroscience 27, 104-107.
Herbert, S. C., et at. (1984) American Journal of Physiology 246, F745-F756.
Hirvonen, M. R., et al. (1991) Neurochemical Research 16, 905-91.1.
Honchar, M. P., et al. (1989) Journal of Neurochemistry 53, 590-594.
Hudson, D. F., et al. (2002) Trends in Cell Biology 12, 281-287.
Inhorn, R. C., et al. (1987a) Proc. Natl. Acad. Sci. USA 84, 2170-2174.
Inhorn, R. C., et al. (1987b) Journal of Biological Chemistry 262, 15946-15952.
Inhorn, R. C., et al. (1988) Journal of Biological Chemistry 263, 14559-14565.
Irvine, R. F., et al. (2001) Nature Reviews 2, 327-338.
Jackson, B. A., et al. (1980) Endocrinology 107, 1693-1698.
Jakes et al. (1986) J Mol Graph 4:12-20
Jakes et al. (1987) J Mol Graph 5:41-48
Jakubowski, H., et al. (1993) Journal of Bacteriology 175, 5469-5476.
Jefferson, J. W. (1990) Journal of Clinical Psychiatry 51 (suppl 8), 4-8.
Jo, I., et al. (1995) Proceedings of the National Academy of Sciences: USA 92, 1876-1880.
Johnson, K. A., et al. (2001) Biochemistry 40, 618-630.
Johnson, R. A., et al. (1989) Molecular Pharmacology 35, 681-688.
Jones and Winistorfer, 1992 Biotechniques 12:528-30
Jope, R. S. (1999a) Molecular Psychiatry 4, 117-128.
Jope, R. S. (1999b) Molecular Psychiatry 4, 21-25.
Jope, R. S., et al. (1997) Molecular Brain Research 50, 171-180.
Jope, R. S., et al. (1992) Biological Psychiatry 31, 505-514.
Jope, R. S., et al. (1994) Biochemical Pharmacology 47, 429-441.
Joulin and Richard-Foy (1995) Eur J Biochem 232:620-626
Kim, H. J., et al. (1995) Drug Metabolism and Disposition 23, 840-845.
Keown et al. (1990) Methods in Enzymology 185:527-537
Klaassen, C. D., et al. (1997) FASEB Journal 11, 404-418.
Klein, P. S., et al. (1996) Proceedings of the National Academy of Sciences: USA 93, 8455-8459.
Kuntz I D et al. (1982) J Mol Biol 161:269-288
Kurima, K., et al. (1998) Proceedings of the National Academy of Sciences: USA 95, 8681-8685.
Laemmli, U. K. (1970) Nature 227, 680-685.
Lam et al. (1994) Science 263:380-384
Li et al. (1996) Cell 85:319-329
Li, H., et al. (1995) Journal of Biological Chemistry 270, 29453-29459.
Liebenhoff, U., et al. (1995) FEBS Letters 365, 209-213.
Lin, W. Y., et al. (1991) Biochemistry 30, 3421-3426.
Lopez, F., et al. (1999) Molecular Microbiology 31, 1255-1264.
Lopez-Coronado, J. M., et al. (1988) Biochimica et Biophysica Acta 939, 467-475.
Lu, R., Song, L., et al. (1999) Neuroreport 10, 1123-1125.
Luyckx et al. (1999) Proc Natl Acad Sci USA 96(21):12174-12179
Mao, C., et al. (2001) Journal of Biological Chemistry 276, 26180-26188.
Marcus, F., et al. (1980) Journal of Biological Chemistry 255, 2481-2486.
Marotti and Tomich, 1989 Gene Anal Tech 6:67-70
Marples, D., et al. (1995) Journal of Clinical Investigation 95, 1838-1845.
Marples, D., et al. (1998) Proceedings of the Association of American Physicians 110, 401-406.
Martin, R. L., (1989) Journal of Biological Chemistry 264, 11768-11775.
Matsuhisa, A., et al. (1995) Journal of Bacteriology 177, 200-205.
Montrose, M. H., et al. (1999) Textbook of gastroenterology (Yamada, T., Ed.) pp 320-354, Lippincott Williams and Willkins, Philadelphia.
Mortlock et al. (1996) Genome Res. 6:327-33
Mori, S., et al. (1998) Neuropsychopharmacology 19, 233-240.
Muller-Oerlinghausen, B., et al. (2002) Lancet 359, 241-247.
Murguia, J. R., (1995) Science 267, 232-234.
Murguia, J. R., et al. (1996) JBC 271, 29029-29033.
Naccarato, W., et al. (1974) Archive of Biochemistry and Biophysics 164, 194-201.
Nahorski, S. R., et al. (1991) Trends in Pharmacological Sciences 12, 297-303.
Nakashima, K., et al. (1976) Journal of Biological Chemistry 251, 4315-4321.
Nemeroff, C. B. (2000) Journal of Clinical Psychiatry 61 (suppl 13), 19-25.
Neumaier, J. F., et al. (2000) In Bipolar disorders: Basic mechanisms and therapeutic implications (Soares, J. C., and Gershon, S., Eds.) pp 545-553, Marcel Dekker, Inc., New York.
Neuwald, A. F., et al. (1992) Journal of Bacteriology 174, 415-425.
Neuwald, A. F., (1991) FEBS Letters 294, 16-18.
Nielsen, S., et al. (1993) Proceedings of the National Academy of Sciences: USA 90, 11663-11667.
Nielsen, S., et al. (1995) Journal of Clinical Investigation 96, 1834-1844.
Oksche, A., et al. (1998) Journal of Molecular Medicine 76, 326-337.
Owyang, C. (1984) Gastroenterology 87, 714-718.
Ozaki, N., (1997) Journal of Neurochemistry 69, 2336-2344.
Ozeran, J. D., et al. (1996a) Biochemistry 35, 3695-3703.
Ozeran, J. D., et al. (1996b) Biochemistry 35, 3685-3694.
Pandol, S. J., et al. (1980) New England Journal of Medicine 302, 1403-1404.
Parthasarathy, L., et al. (2001) Molecular cloning and expression of human myo-inositol monophosphatase a3 cDNA (impa3). unpublished.
Paszewski, A., et al. (1992) Current Genetics 22, 273-275.
Patel, S., et al. (2002) Journal of Molecular Biology 315, 677-685.
Paul, C. P., et al. (2002) Nature Biotechnology 20, 505-508.
Peng, Z., et al. (1995) Journal of Biological Chemistry 270, 29105-29110.
Phiel, C., et al. (2001) Annual Review of Pharmacology and Toxicology 41, 789-813.
Pollack, S. J., et al. (1994) Proc. Natl. Acad. Sci. USA 91, 5766-5770.
Pollack, S. J., et al. (1993). Eur. J. Biochem 217, 281-287.
Post, S. R., et al. (2000) In Methods in cell biology: Adrenergic receptor protocols (Machida, C. A., Ed.) pp 363-374, Humana Press, Totowa, N.J.
POV-Team (2002) Persistence of Vision™ Ray Tracer, Williamston, Australia.
Prentid et al., 1984 Gene 29:303-13
Pritchard, J. B., et al. (1982) In Metabolic basis of detoxication: Metabolism of functional groups (Jakoby, W. B., Bend, J. R., and Caldwell, J., Eds.) pp 339-357, Academic Press, NY.
Quintero, F. J., et al. (1996) Plant Cell 8, 529-537.
Ramaswamy, S. G., et al. (1987) J. Biol. Chem. 21, 10044-10047.
Rasenick, M., et al. (1996) Journal of Clinical Psychiatry 57 (Suppl 13), 49-55.
Rauchman, M. I., et al. (1993) American Journal of Physiology 265, F416-F424.
Raybould, H. E., et al. (1999) In Textbook of gastroenterology (Yamada, T., Ed.) pp 2-10, Lippincott Williams and Wilkins, Philadelphia.
Reed, D., et al. (1980) Journal of Physiology 309, 329-339.
Rybakowski, J. (2000) Pharmacopsychiatry 33, 159-164.
Ryves, W. J., et al. (2001) Biochemical and Biophysical Research Communications 280, 720-725.
Sands, J. M. (1995) In Textbook of nephrology (Massry, S. G., and Glassock, R. J., Eds.) pp 108-113, Williams & Wilkins, Baltimore.
Sanger, F., et al. (1977) Proceedings of the National Academy of Sciences: USA 74, 5463-5467.
Sambrook et al., 1989 Molecular Cloning: A Laboratory Manual, CSH Press, pp. 15.3-15.108
Sauer (1998) Methods 14(4):381-392
Sauer, B. (1998) Methods 14, 381-392.
Sayers et al., 1992 Biotechniques 13:592-6
Scott & Smith (1990) Science 249:386-390
Schneider, B., et al. (1998) Journal of Biological Chemistry 273, 28773-28778.
Schou, M. (2001) Journal of Affective Disorders 67, 21-32.
Schriever, C., et al. (1997) Biological Chemistry 378, 701-706.
Scott, J., et al. (2002) Journal of Clinical Psychiatry 63, 384-390.
Sheline, Y. I., et al. (1999) Journal of Neuroscience 19, 5034-5043.
Sheline, Y. I., et al. (1996) Proceedings of the National Academy of Sciences: USA 93, 3908-3913.
Sherman, W. R., et al. (1981) Journal of Neurochemistry 36, 1947-1951.
Sherman, W. R., et al. (1985) Journal of Neurochemistry 44, 798-807.
Sikorski, R. S., et al. (1989) Genetics 122, 19-27.
Singer, S. S. (1979) Analytical Biochemistry 96, 34-38.
Skinner, G. (1983) Lancet 2, 288.
Skinner, G., et al. (1980) Medical Microbiology and Immunology 168, 139-148.
Snyder, H. M., et al. (1992) American Journal of Physiology 263, C147-C153.
Soares, J. C., et al. (1997) Biological Psychiatry 41, 86-106.
Spector, R., et al. (1975) American Journal of Physiology 228, 1510-1518.
Spiegelberg, B. D., et al. (1999) Journal of Biological Chemistry 274, 13619-13628.
Stambolic, V., et al. (1996) Current Biology 6, 1664-1668.
Stanton, B. A., et al. (1998) In Physiology (Berne, R. M., and Levy, M. N., Eds.) pp 677-698, Mosby, Inc., St. Louis.
Stec, B., et al. (2000) Nature Structural Biology 7, 1046-1050.
Stepinski, J., et al. (1984) Acta Biochimica Polonica 31, 229-240.
Stieglitz, K. A., et al. (2002) Journal of Biological Chemistry 277, 22863-22874.
Stolz, L. E., et al. (1998a) Genetics 148, 1715-1729.
Stolz, L. E., et al. (1998b) Journal of Biological Chemistry 273, 11852-11862.
Sunden, S. L. F., et al. (1997) Archives of Biochemistry and Biophysics 345, 171-174.
Sylvia, V., et al. (1988) Cell 54, 651-658.
Thomas, D., et al. (1992) Journal of General Microbiology 138, 2021-2028.
Thomas & Capecchi (1990) Nature 346(6287):847-850
Uchida, S., et al. (1994) Journal of Biological Chemistry 269, 23451-23455.
Ullrich, T. C., et al. (2001a) The EMBO Journal 20, 316-329.
Ullrich, T. C., et al. (2001b) Journal of Molecular Biology 313, 1117-1125.
Unlap, M. T., et al. (1997) Neuropsychopharmacology 17, 12-17.
Vawter, M. P., et al. (2000) Biological Psychiatry 48, 486-504.
Venkatachalam, K. V., et al. (1998) Journal of Biological Chemistry 273, 19311-19320.
Wade, J. B., et al. (1981) New York Academy of Sciences 81, 106-117.
Weiner et al., 1993 Gene 126:35-41
West & Fairlie (1995) Trends Pharmacol Sci 16:67-74
Whitworth, P., et al. (1990) British Journal of Pharmacology 101, 39-44.
Whitworth, P., et al. (1989) Journal of Neurochemistry 53, 536-541.
Williams, M. B., et al. (1995) Psychopharmacology 122, 363-368.
Williams, R. S. B., et al. (2002) Nature 417, 292-295.
Willner, P. (1995) In Psychopharmacology: The fourth generation of progress (Bloom, F. E., and Kupfer, D. J., Eds.) pp 921-931, Raven Press, New York.
Winzeler, E. A., et al. (1999) Science 285, 901-906.
Wlodawer & Erickson (1993) Ann Rev Biochem 62:543-585
Wong, Y.-H. H., et al. (1987) Journal of Neurochemistry 48, 1434-1442.
Woods, S. W. (2000) Journal of Clinical Psychiatry 61 (suppl 13), 38-41.
Wright, E. M. (1978) Reviews of Physiology, Biochemistry, and Pharmacology 83,1-34.
Wright, E. M., et al. (1986) Annals of the New York Academy of Science 481, 214-220.
Wyatt, R., et al. (1995) Social Psychiatry and Psychiatric Epidemiology 30, 213-219.
Wyss-Coray et al. (1995) Am. J. Pathol. 147:53-67
Yamaki, M., et al. (1991) Am J Physiology 261, F505-F511.
Yang, T., et al. (1996a) American Journal of Physiology 271, F931-F939.
Yang, Y.-S., et al. (1996b) Protein Expression and Purification 8, 423-429.
York, J. D., et al. (1994a) J. Mol. Biol. 236, 584-589.
York, J. D., et al. (1994b) Biochemistry 33, 13164-13171.
York, J. D., et al. (1995) Proc. Natl. Acad. Sci. USA 92, 5149-5153.
York, J. D., et al. (1994c) J. Biol. Chem. 269, 19992-19999.
York, J. D., et al. (1993) Proc. Natl. Acad. Sci. USA 90, 5833-5837.
Yoshikawa, T., et al. (2000) Molecular Psychiatry 5, 165-171.
Yoshikawa, T., et al. (1997) Molecular Psychiatry 2, 393-397.
Young, L. T. (2001) Journal of Psychiatry and Neuroscience 26 (Suppl), S17-S22.
Yuan, P. X., et al. (1998) Molecular Brain Research 58, 225-230.
Zhu 1989 Anal Biochem 177:120-4

It will be understood that various details of the presently disclosed subject matter can be changed without departing from the scope of the presently disclosed subject matter. Furthermore, the foregoing description is for the purpose of illustration only, and not for the purpose of limitation.

1. A method for identifying a compound that modulates the activity of a PAP phosphatase enzyme, comprising:
(a) contacting a compound with a PAP phosphatase polypeptide; and
(b) detecting modulation of the activity of said PAP phosphatase polypeptide.

2. The method of claim 1, comprising selecting said compound as a PAP phosphatase modulating compound if said compound modulates the activity of said PAP phosphatase polypeptide.

3. The method of claim 1, wherein said PAP phosphatase polypeptide is a BPntase polypeptide.

4. The method of claim 2, wherein contacting said compound with said PAP phosphatase polypeptide comprises growing at least one recombinant yeast strain expressing said PAP phosphatase polypeptide in a minimal media lacking methionine and containing said compound, such that said compound contacts said recombinant yeast.

5. The method of claim 4, wherein said recombinant yeast strain is selected from the group consisting of ha/2::Hal2p, ha/2::BPntase, and combinations thereof.

6. The method of claim 5, wherein said ha/2::BPntase strain is a ha/2::hBPntase.

7. The method of claim 4, wherein detecting modulation of the activity of said PAP phosphatase polypeptide comprises measuring for growth of said recombinant yeast strain.

8. The method of claim 7, wherein measuring for growth of said recombinant yeast strain comprises measuring a change in optical density.

9. The method of claim 7, wherein selecting said compound as a PAP phosphatase modulating compound comprises selecting said compound as said PAP phosphatase modulating compound if growth of said recombinant yeast strain is inhibited.

10. The method of claim 9, wherein said yeast strain is a combination of said hal2::Hal2p strain and said hal2::BPntase strain grown concurrently in minimal media lacking methionine and containing said compound and said compound is selected as said PAP phosphatase modulating compound if at least growth of said hal2::BPntase yeast strain is inhibited.

11. The method of claim 1, wherein detecting modulation of the activity of said PAP phosphatase polypeptide comprises detecting binding of said compound to said PAP phosphatase polypeptide.

12. The method of claim 11, wherein said PAP phosphatase polypeptide is a mammalian BPntase polypeptide.

13. The method of claim 12, wherein said compound binds at an active site of said BPntase enzyme.

14. The method of claim 13, wherein said active site is a lithium binding site.

15. The method of claim 13, wherein said active site is a low affinity Mg²⁺ binding site.

16. The method of claim 1, wherein detecting modulation of the activity of said PAP phosphatase polypeptide comprises detecting inhibition of the activity of said PAP phosphatase polypeptide.

17. The method of claim 16, wherein said PAP phosphatase polypeptide is a mammalian BPntase polypeptide.

18. A method for identifying a compound that modulates the activity of a sulfur assimilation pathway enzyme, comprising:
(a) contacting a compound with a sulfur assimilation pathway enzyme; and
(b) detecting modulation of the activity of said sulfur assimilation pathway enzyme.

19. The method of claim 18, wherein said sulfur assimilation pathway enzyme is selected from the group consisting of ATP sulfurylase, APS kinase, sulfotransferase, PAPS reductase, PAP phosphatase and combinations thereof.

20. The method of claim 19, wherein said sulfur assimilation pathway enzyme is a yeast sulfur assimilation pathway enzyme.

21. The method of claim 20, wherein said ATP sulfurylase enzyme is a Met3 enzyme and said APS kinase enzyme is a Met14 enzyme.

22. The method of claim 19, wherein said sulfur assimilation pathway enzyme is a mammalian sulfur assimilation pathway enzyme.

23. The method of claim 22, wherein said ATP sulfurylase enzyme and said APS kinase enzyme together are a bifunctional PAPS synthetase enzyme.

24. The method of claim 18, wherein detecting modulation of the activity of said sulfur assimilation pathway enzyme comprises detecting binding of said compound to said sulfur assimilation pathway enzyme.

25. The method of claim 18, wherein detecting modulation of the activity of said sulfur assimilation pathway enzyme comprises detecting inhibition of the activity of said sulfur assimilation pathway enzyme.

26. The method of claim 18, wherein detecting modulation of the activity of said sulfur assimilation pathway enzyme comprises detecting a change in the amount of a sulfur assimilation pathway enzyme product.

27. The method of claim 26, wherein said sulfur assimilation pathway product is selected from the group consisting of APS, PAPS, PAP, AMP, cAMP, and combinations thereof.

28. The method of claim 18, wherein detecting modulation of the activity of said sulfur assimilation pathway enzyme comprises detecting a change in the amount of a sulfur assimilation pathway enzyme substrate.

29. The method of claim 28, wherein said sulfur assimilation pathway enzyme substrate is selected from the group consisting of ATP, APS, PAPS, PAP, AMP, and combinations thereof.

30. A transgenic non-human vertebrate animal having incorporated into its genome a modified gene encoding a BPntase polypeptide.

31. The transgenic animal of claim 30, wherein said modified gene encodes a biologically active human BPntase polypeptide.

32. The transgenic animal of claim 31, wherein said modified gene is incorporated into said genome so as to confer overexpression in said animal of said biologically active human BPntase polypeptide.

33. The transgenic animal of claim 30, wherein said modified gene is disrupted wherein said disrupted modified gene results in one of expression of a nonfunctional BPntase polypeptide and substantially no expression of a BPntase polypeptide.

34. The transgenic animal of claim 30, wherein expression of said BPntase polypeptide is conferred in a tissue or blood of said transgenic animal.

35. The transgenic animal of claim 34, wherein said tissue is selected from the group consisting of kidney tissue, brain tissue, liver tissue, intestinal tissue, skin tissue, heart tissue, lung tissue, spleen tissue, bone marrow, and combinations thereof.

36. The method of claim 33, wherein said disruption of said gene is a homozygous disruption.

37. A transgene construct comprising an isolated BPntase gene encoding a BPntase polypeptide cloned into a vector.

38. The transgene construct of claim 37, wherein said vector is a plasmid.

39. An isolated cell comprising said transgene construct of claim 38.

40. A method of identifying a compound for treating a toxic effect resulting from a therapeutic treatment, comprising:
(a) obtaining a transgenic non-human vertebrate animal having incorporated into its genome a disruption of a gene encoding a BPntase polypeptide, wherein said disruption results in said transgenic animal exhibiting said toxic effect;
(b) administering said compound to said transgenic animal; and
(c) observing said transgenic animal for a change in said transgenic animal indicative of amelioration of said effect.

41. The method of claim 40, wherein said therapeutic treatment is a lithium treatment for a neurological disorder.

42. The method of claim 41, wherein said neurological disorder is bipolar disorder.

43. The method of claim 40, wherein said disruption of said gene is a homozygous disruption.

44. The method of claim 40, wherein disruption of a gene encoding a BPntase polypeptide is conferred to a tissue or blood of said transgenic animal.

45. The method of claim 44, wherein said tissue is selected from the group consisting of kidney tissue, brain tissue, liver tissue, intestinal tissue, skin tissue, heart tissue, lung tissue, spleen tissue, bone marrow, and combinations thereof.

46. The method of claim 40, wherein said transgenic animal is a mouse.

47. The method of claim 40, wherein said toxic effect is selected from the group consisting of emesis, diarrhea, organ dysfunction, hypothyroidism, and combinations thereof.

48. The method of claim 47, wherein said organ dysfunction is kidney dysfunction.

49. A method for treating lithium-related toxicity, comprising administering to a subject suffering from said toxicity a therapeutically effective amount of a compound that modulates the activity of at least one sulfur assimilation pathway enzyme.

50. The method of claim 49, wherein said method comprises treating a lithium-related toxic effect selected from the group consisting of nausea, emesis, diarrhea, organ dysfunction, hypothyroidism, and combinations thereof.

51. The method of claim 50, wherein said organ dysfunction is kidney dysfunction.

52. The method of claim 49, wherein said sulfur assimilation pathway enzyme is selected from the group consisting of ATP sulfurylase, APS kinase, sulfotransferase, PAPS reductase and combinations thereof.

53. The method of claim 49, wherein said sulfur assimilation pathway enzyme is a mammalian sulfur assimilation pathway enzyme.

54. The method of claim 53, wherein said ATP sulfurylase enzyme and said APS kinase enzyme together are a bifunctional PAPS synthetase enzyme.

55. The method of claim 49, wherein said compound is chlorate.

56. A method of identifying a compound that modulates the activity of a BPntase polypeptide, comprising modeling an interaction between said compound and a target moiety on said BPntase polypeptide.

57. The method of claim 56, wherein modeling is computer modeling.

58. The method of claim 56, wherein said interaction is binding of said compound to said BPntase polypeptide by hydrogen bonding, van der Waal's binding, or both hydrogen bonding and van der Waal's bonding.

59. The method of claim 56, wherein said target moiety is a druggable region.

60. The method of claim 59, wherein said druggable region is a lithium binding site.

61. The method of claim 60, wherein said target moiety is a low affinity Mg²⁺ binding site.

62. The method of claim 61, wherein said target moiety is a cluster of amino acid residues fixed at specific spatial points as determined by secondary structure of said BPntase peptide, said residues being Asp-51, Glu-74, Asp-117, and Leu-119.


SEQUENCE LISTING (24 sequences)

SEQ ID NO: 1 - DNA, 2113 bp, Homo sapiens
  mRNA (1)..(2113)
ggaattcggcacgagaagctcggtactggacacaacgagggacctgggtctacgataacgcgcttttgctcctcctgaagtgtctttggtccaacgttgttccagagtgtaccatggcttccagtaacactgtgttgatgcggttggtagcctccgcatattctattgctcaaaaggcaggaatgatagtcagacgtgttattgctgaaggagacctgggtattgtggagaagacctgtgcaacagacctgcagaccaaagctgaccgattggcacagatgagcatatgttcttcattggcccggaaattccccaaactcacaattataggggaagaggatctgccttctgaggaagtggatcaagagctgattgaagacagtcagtgggaagaaatactgaagcaaccatgcccatcgcagtacagtgctattaaagaagaagatctcgtggtctgggttgatcctctggatggaaccaaggaatataccgaaggtcttcttgacaatgtaacagttcttattggaattgcttatgaaggaaaagccatagcaggagttattaaccagccatattacaactatgaggcaggaccagatgctgtgttggggaggacaatctggggagttttaggtttaggcgcctttgggtttcagctgaaagaagtccctgctgggaaacacattatcacaactactcgatcccatagcaacaagttggttactgactgtgttgctgctatgaaccccgatgctgtgctgcgagtaggaggagcaggaaataagattattcagctgattgaaggcaaagcctctgcttatgtatttgcaagtcctggttgtaagaagtgggatacttgtgctccagaagttattttacatgctgtgggaggcaagttaaccgatatccatgggaatgttcttcagtaccacaaggatgtgaagcatatgaactctgcaggagtcctggccacactgaggaattatgactactatgcaagccgagttccagaatctattaaaaatgcacttgttccttaaaggaaagtttcatttggccgggcgcggtggctcatgcctgtaatcccagcactttgggaggccgaggcaggtggatcacttgagctcaggagtttgagaccagcctgggcaatatcgtgagaccccatctctacaaaaatacaaattaactgggcatcctgtcatgcgcctgtcatcccagctacttgagaggctgaagcagaagaatctcttgagcccggaaggcggaggttgcagtgagctgagatcgtgccactgcactccagcctgagtgacaggagttaagccctgtctcagaaaaaaaacataaacccaaaaagtacttaaagtttcatttacttactaggaaaagacttggttctcaaataatacattttaagattaattgggtagaattagagttccacctttatcattgttgacagtgatttatatttagttatatatttagaataaaaattaactaaataatttaacttgattaataccattactcaacctgacaattgagttggagacttataaactcattatggttatcatgtgttttcctgttgaatgtgaagaagtgagaaaacatttgccaatgacagttaggcgtgcacactgaccattcactgataaaccagattctgcctgaatctgaagggattgcttgtagcatagggtttagtggcgtgatcttgggtcactgcggcccgcttccggggttcatgcttctcctgcctagctccgggtagctgggactgcagcacggcccacgctggtaattttttgtatgatggtgagaagttttcaccgtgttgccaggatggcttatcctgacatcgtgatctgtatgcctcggatcccaaagtgcatgggatgacagctgtgagccaccgcacttggcttaaaccagatttctttagggcacatttttttggaatctcactctgtttttcacagtaattttaaaaacgttttatccaattagaatatatatgatgttattatatatgcttatgaaacagatttatgagaaaagttttttttaaataaattatttaatccctaaaaaaaaaaaaaaaaaa

SEQ ID NO: 2 - protein, 308 aa, Homo sapiens
  PEPTIDE (1)..(308)
MASSNTVLMRLVASAYSIAQKAGMIVRRVIAEGDLGIVEKTCATDLQTKADRLAQMSICSSLARKFPKLTIIGEEDLPSEEVDQELIEDSQWEEILKQPCPSQYSAIKEEDLVVWVDPLDGTKEYTEGLLDNVTVLIGIAYEGKAIAGVINQPYYNYEAGPDAVLGRTIWGVLGLGAFGFQLKEVPAGKHIITTTRSHSNKLVTDCVAAMNPDAVLRVGGAGNKIIQLIEGKASAYVFASPGCKKWDTCAPEVILHAVGGKLTDIHGNVLQYHKDVKHMNSAGVLATLRNYDYYASRVPESIKNALVP

SEQ ID NO: 3 - protein, 364 aa, Artificial
  Li-sensitive sequence uniting motif.
DXXXXXXXXXXXXXXXXXXXXXXXXXXXXXXXXXXXXXXXXEEXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXDPXDXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXGG

SEQ ID NO: 4 - protein, 290 aa, Artificial
  Li-sensitive sequence uniting motif for Impase1.
XXXXXXXXXXXXXXXXXXXXXXXXXXXXXXXXXXXXXXXXXXXXXXDXXXXXXXXXXXXXXXXXXXXXXEEXXXXXXXXXXXXXXXXXXDPIDGTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXWDXXXXXXXXXXXGGXXXXXXXXXXXXXXXXXXXXXXXXXXXXXXXXXXXXXXXXXXXXXXXXXXXXXXXXX

SEQ ID NO: 5 - protein, 399 aa, Artificial
  Li-sensitive sequence uniting motif for 1ptase.
XXXXXXXXXXXXXXXXXXXXXXXXXXXXXXXXXXXXXXXXXXXXXXXXXXXXXDXXXXXXXXXXXXXXXXXXXXXXXXEEXXXXXXXXXXXXXXXXXXXXXXXXXXXXXXXXXXXXXXXXXXXXXXXXXXXXXXXXXXXXXXXXXXXXXXXXDPIDSTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXWDXXXXXXXXXXXGGXXXXXXXXXXXXXXXXXXXXXXXXXXXXXXXXXXXXXXXXXXXXXXXXXXXXXXXXXXXXXXXXXXXXXX

SEQ ID NO: 6 - protein, 338 aa, Artificial
  Li-sensitive sequence uniting motif for Fbpase1.
XXXXXXXXXXXXXXXXXXXXXXXXXXXXXXXXXXXXXXXXXXXXXXXXXXXXXXXXXXXXXXXXXXXXXXXXXXDXXXXXXXXXXXXXXXXXXXXXXEEXXXXXXXXXXXXXXXXXXXDPLDGSXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXYEXXXXXXXXXXXGGXXXXXXXXXXXXXXXXXXXXXXXXXXXXXXXXXXXXXXXXXXXX

SEQ ID NO: 7 - DNA, 53 bp, artificial
  5′ DNA PCR primer.
ggatccgagctcgaattccaccatggagatccccgggagcctgtgcaagaaag

SEQ ID NO: 8 - DNA, 55 bp, artificial
  5′ DNA PCR primer.
ggatccgtcgacgagctcgcggccgcggtggagtgactgggttaacagcctaagc

SEQ ID NO: 9 - DNA, 49 bp, artificial
  5′ DNA PCR primer
agatctttcaattgaagcttgtcgaccagcatgtcggggatcaagaagc

SEQ ID NO: 10 - DNA, 48 bp, artificial
  5′ DNA PCR primer.
agatctaagcttccgcggtcgacctggagccaaaggcttagttcttct

SEQ ID NO: 11 - DNA, 31 bp, artificial
  5′ DNA PCR primer
ggatccatgcctgctcctcacggtggtattc

SEQ ID NO: 12 - DNA, 43 bp, artificial
  5′ DNA PCR primer.
ccgcggtcgacgcggccgcggtcgatcatgaattttgccctac

SEQ ID NO: 13 - DNA, 31 bp, artificial
  5′ DNA PCR primer.
ggatccaagcacactgtacaccaatggctac

SEQ ID NO: 14 - DNA, 42 bp, artificial
  5′ DNA PCR primer.
gcggccgccgcggtcgaccggatcagaatttcacggtaatcc

SEQ ID NO: 15 - DNA, 31 bp, artificial
  5′ DNA PCR primer.
atcgatcatatggagcccttgcgtaaaccac

SEQ ID NO: 16 - DNA, 22 bp, artificial
  5′ DNA PCR primer
tcatatttgacagcggaacgtg

SEQ ID NO: 17 - DNA, 981 bp, artificial
  Fragment of BPntase genomic DNA located between exons 5 and 6 with 5′ and 3′ engineered restriction sites.
gtagcacctcacatactctcccagctccagagctaggcccctcctggggaatcactgttgtacacttcctttcctgagggactgtgctgacatgtctgactgggctagagaaatgctccaccacccctggtcccatagcatcccctcacctgaggttgtcacaggtaagaaaaccagaaggcatcgaattaaatccagaggtgtaaaagtcaggaggagttgtgtgagagctcacacctgtaatctcagcacactggggcagagggactgctttgagtttgaggccatcttgagtgctatacatggcaagttctgggtcagcttgggttagagcaagaccttttctaggcaaagcaagacattagtcagaagaacccagtctcagagctggacttcgggttttatttgtttgtttgtttgtttttattttttgagacagggtttctctgtgtagccctggttgtcctggcactcactttgtagaccagactggcctcgaactcagaaatctgcctgcctctgcctcccgagtgctgggattaaaggtgtgcgccaccactgcctggcttagacttcaagttttaaaagcctagagttgtagttttgaaataaagatctgcattgagaacttgtgaggctgaggcaggaagactgtgaggtcagcctggccttcacagtgagtttcaggtcagcctgagatagaggagcagtgtgaggccagaaggaccccacaaagaaagacctccacagcgctgcttctaacgggtccagcttcgagaggctttctcacagctgccagagagaatgttgttggcccctggaggagatagagtgatagtgactctgtgtgtgtgtgtgtaaatatatactgtatatactgtgagggtgcatgtgtgcaacatgcatatgtactgtgaaaatgtgtgagaggcagtgtgttcgtatgtgtgtctgtgagtataacccatgcgtatgtaatct

SEQ ID NO: 18 - DNA, 35 bp, artificial
  5′ DNA PCR primer
ggcgcgccgtagcacctcacatactctcccagctc

SEQ ID NO: 19 - DNA, 34 bp, artificial
  3′ DNA PCR primer
ggcgcgccagattacatacgcatgggttatactc

SEQ ID NO: 20 - DNA, 4858 bp, artificial
  Fragment of the genomic BPntase sequence with engineered 5′ and 3′ flanking restriction sites.
tggcgagcttgcttattctgctttcagagtatggggttgtataaagcacgtggcgccacactggggtttcccgactcttagcccatttaaagcaggttggaatctagagcgttatgaaagagtttctcaattagagaagagaatattccaaataatttaaaagcacctttgcaaacttgaactgttgtggagctgggaatgcagttcagtggtagtgcctgcttggtgtgtgtcaggggcgtgtgtgatctctatcagcacacacacacacacacacacgcacgcacgcacgcacacacacacgcacgcacacacacacgcacacacgcacgcacacacgcgcgcacacacacacacacacacacacacacacacacgctgttttaaactatgattgtttattggtatacagtttcacacggtagtacaagctgatctcagattcatggctgtccttctatgtcctgatattagagccgtgcagtgctatccagcctcacttctcagtcttttttgttttttgttttgttttgttttgttttgttttatttttttgagacagggtttctctgtgaagccctggctgtcctggaactcactctgtagaccaggctggcctcgaactcagaaatccgcctgcctctgcctcccgagtgctgggattaaaggcgtgcgccgccacgcccggcgtcacttctcagtcttagctgctgttacttctctgagaagcagcgagggccctcactagttgatccctgggctcgggtctgcgttatactggggagtcggaagactggttaccccgatttgtactgatacggagatttgcattcttggttacagacctcggccaccgacctgcagaccaaagccgaccgcttggtgcagatgagcatatgctcttccctggcccggaagttcccgaagctgaccatcataggggaagaggtgagaggcgcgcgccacttggattcataccctacctgccattgagccgtaggttatggtcagtcttagcgttggcactaacgttccaacacaaagcgatcgttttccttaggggaaaaatctgacttaatgatattttggtccacttaatgggctaagtctccatttctagtgatgggagctatggtcaccattgtaataccatacgatggactcagtggcagaaagtcgcctactgtatgtgaggctctaaggtgggagacatctcagtcataagaccatgtggctcacatatgtgaggtcctgggttggagcccctgcatcagcagttatatgtgaagagtcggcaaggttctggaactctgagatgactgggcttggtttgcttgtctgcttgtccgtcatttcagattggacttgtgtacttacaactgaaacaatagactatgttttagttttgttttttattaagttagttcgttgacaatttagtgcatacatgtaatacattctgatttccctcatacttcggattctcctcccctccccttcttccttgcccgccccctcttcccacttagtttattcaggatcatccatgtgaccatttcattgggaccatccattggtcatcagtggtgcacagctgaaagcaatggcttcccctttccctgaatcagtctgtaggaaatagttctgcagtgaaggagagagagtgctggtctgcatctctcctccacctctgcttaactgttgggactcattctttctcagacccagcacagtcatctggttgttgagagttcatgcttgcactggctcgcaccccagtaatgacgtttggcagcccttctccccgtttttcagctcttaccatctttctgcccctctcctacaaagcctggtaaaccttagaggggataaatgtctaaatatcttattcagagctgagcaatcagctgtaagtttgtcttattaggccttcatatatctctcccttcattatagtcctctagaaagagaatcttctctgactaaggctgagtggtaattcgctatgtgaataaacatctatatttaggaagctgtttgacactgtgtgactttagtaaagctgtagagtttaactccctaagaggactcatggcctccctttttatacactgagtgggtctccagacatggagtgtgtttaacgtactaagcgtggattcccatgctggagtagccctcacattcgatcaagagcaggtagttaccccccaacagtgccgacactgttgttgtaccagtgagcacagcttgcctgacagatggtgctgtagtttgtcaggtgcacagatgggcaatactttcttccccagcagcctgcagagaaaatgtgttcaggtctgacttctttgtctcatgcaaccaaagtgtgtggtgtcattagcagtaaggtcttagcatctaatgctagtgggcaaccaagaaaaatgacaatgcctatattgtcttagggcagtgggacctccgtgaccaacttatcaggaggcaccacacacacagcaggtggggttttaatgaaggataatttcacaggggagcagtttctaggtctctctcttccaacttaaaaaaatgcatcctagttattgtgagtaaattgaaaatcaacagataagttagtttccaacagtgcgatgtcaggcctctggacgtgtggaagacagcagtattccatgtactgggatagctggccatgtgccggaacagctgggctacggatgctgttcttagtgttgtaaggaattgccacaccagtttccatatggctgcactggtttcccaccagcaatgaaggagtccctcttttccaccctcaccagcactgcctgtcttgaggtttcttacggattgccattctgacaggacaagatgaaatcttagagcagctttaatttgtacttccttttgtgctaatgatgtcaaatactttttaaaatgtttatttttcaatcctattacttttgagaattctctgttcagttccatagcccatttttgctgttgtttgttgacacagggtctttctctatgtatccctggctatgtagaacagagttacctcaaagctgacagagatccatctacttttacctcccaggtgctgggattagaggcatgcagatttttaaagttattaatatttatttgtgtacctgtgtctgatctgtgtatgtggatggatgttttgcttgcctggatagctctgctccacatgtgagccagttacctgcggtggccagaagagggcatcggatgccctggaactggagttaggatggttgtgagctgcctgtgggtgctcgagaacaagcctgggtcctcgggagagcagcgttgttcttacctcctagcccactctccagctccgggggttgattcttgttcaagacagcagagaaggctcgagcttccctcttctccgtgtagacatccagtcttcccagcgccagtttgagatgctccctctcccttcgtgtatttttggtgtgtttttcaagaatcaggtggctgtaattgtatggcattagtccggggtttccattctgttgcactgatctacacatcggtttttgtgccagcaccgtgccctttgttaccctgattctgtagtgttattttggctcagaatttttttggctgcctgggccttttgtgttttgcatcactcattctaccgatccatgagcagggagacctttcatctactagtctctgccttgatttctttctttagagttttttgagacagggtctcacatataatcttggctgaccaggaactcagtgtgtagaccaggctggcctcaaactcacagagttctgcctgcctctgccccctgagtgctggggtttaaggcttatgccactgggcctgggattttctctgattttaaagttttcattgtagaggttcttcacttctttgcttgggtttcctctgaggtactttgtttattaaggctgctataaatgggattgtttttctgatttcttcttcaccttgtttgccattggtataaaaaagcatgccatgtgtgtatcctgacactgcagaagtgttagtaattctaggagctttctggtggagactgtagggtctcccatgtacagcattatattctctgtagacaggaacacgctgtcttctttatttcctacctatattcctttcccatcttgtttttattgttttagctaagactaaaacaccaaatcgatggccctgtctcgcttctagttttaatgttgatgtttgtttggcagggtctcgttacttagcccaggctggccttgaattcttcctgcttcatccaacccagtgctgggattaccagtacacaggactctataaaaaggtttttgtttttgttattgtttttatttctctcttctctgcactcacattgcccctctggtgctggagatcatgccagagtctcgtgtgtgctgggcaattactgaccactgagctggatcccaggtcccttgtgtaactcaatgccaagttcattcccactgtctcagcctcccagcattccaaaggaaattggggaaacagaaatatgtaaaggaaactggatgtatttacaattttaggtaaacagatatgaggaaaaggttttgggttccttctagacgttcctgagtcagggtttacatgtggctaggacccagccgtgaggctttctgtgaggatgctgttcctcgtgctcatacttcaaactaggatggaagctcctgggccaatcctagctgctcacttcctctctttcggctcctgccagtcttatgctaggtttgctataaaagctctaaatattagagaaattaaacagaagtggctaggcgtcccattgct

SEQ ID NO: 21 - DNA, 35 bp, artificial
  PCR DNA primer
gcggccgctggcgagcttgcttattctgctttcag

SEQ ID NO: 22 - DNA, 32 bp, artificial
  PCR DNA primer
aagcttagcaatgggacgcctagccacttctg

SEQ ID NO: 23 - DNA, 24 bp, artificial
  oligonucleotide for PCR screen
tccagccttgggacaagagatcag

SEQ ID NO: 24 - DNA, 24 bp, artificial
  DNA oligonucleotide for PCR screen
accaaagaacggagccggttggcg